(19) United States
(12) Patent Application Publication
(10) Pub. No.: US 2015/0190083 A1
(43) Pub. Date: Jul. 9, 2015

(54) Biomarkers For Treatment Outcomes

(71) Applicant: BRC Operations Pty Limited, Ultimo (AU)

(72) Inventors: Evian Gordon, Vaucluse (AU); Leanne Williams, San Francisco, CA (US); Donna Palmer, Glebe (AU); William Rekshan, Oakland, CA (US)

(21) Appl. No.: 14/557,132

(22) Filed: Dec. 1, 2014

Related U.S. Application Data
(63) Continuation of application No. PCT/AU2013/000575, filed on May 31, 2013.
(60) Provisional application No. 61/765,009, filed on Feb. 14, 2013.
(60) Provisional application No. 61/757,595, filed on Jan. 28, 2013.
(60) Provisional application No. 61/757,591, filed on Jan. 28, 2013.

(30) Foreign Application Priority Data
Jun. 1, 2012 (AU) 2012902294

(51) Int. Cl. A61B 5/16; C12Q 1/68

(52) U.S. Cl. CPC A61B 5/165; C12Q 1/6883; A61B 5/162; C12Q 2600/156; C12Q 2600/158; C12Q 2600/106

(57) ABSTRACT

The utilization of biomarkers, which include objectively measurable and assessable indicators of a biological process or biological state, in treatments is disclosed. Particularly, biomarkers enabling optimisation of treatment regimes and the use of the biomarkers in tests for the prediction of optimised treatments and treatment outcomes in the treatment of Major Depressive Disorder (MDD) are disclosed. Measurable and assessable cognitive and/or genomic parameters can be biomarkers indicating changes in system severity of MDD.

### FIELD OF THE INVENTION

The present invention relates to the field of biomarkers and in particular to their utilisation in treatment. Embodiments of the invention have been particularly developed as biomarkers enabling optimisation of treatment regimes and as uses of the biomarkers in tests for the prediction of optimised treatments and treatment outcomes in the treatment of Major Depressive Disorder (MDD). The invention will be described hereinafter with reference to this application. However, it will be appreciated that the invention is not limited to this particular field of use.

### BACKGROUND

Any discussion of the background art throughout the specification should in no way be considered as an admission that such art is widely known or forms part of common general knowledge in the field.

Major depressive disorder (MDD) affects 121 million people worldwide. It has the highest burden of illness in high-income countries based on disability-adjusted life-years, is the third most disabling medical condition worldwide and is the second-ranked cause of lost quality of life in persons aged 15 to 44 years and is expected to move to second by 2020 (World Health Organization. Mental Health: Mental Health Atlas 2011. Switzerland: World Health Organization, 2011). The economic burden of depression in the United States in 2000 amounted to roughly 83 billion US dollars, 31% of which were attributed to direct medical costs (Greenberg et al. 2003).

Antidepressant medications (ADMs) are the most commonly-used treatment for depression, especially in primary care. ADMs are effective (American Psychiatric Association. Practice guideline for major depressive disorder in adults. Am J Psychiatry 1993; 150(4 Suppl.):1-26; Depression Guideline Panel. Clinical practice guideline. Number 5. Depression in primary care: volume 2. Treatment of major depression. Rockville, Md.: U.S. Department of Health and Human Services, Public Health Service, Agency for Health Care Policy and Research, 1993; Frank E. et al., 1993; Thase, M. E. and Rush, A. J. Treatment-resistant depression. In: Bloom F E, Kupfer D J, eds. Psychopharmacology: fourth generation of progress. New York: Raven Press, 1995:1081-97; Rosenbaum J. F. et al., Treatment-resistant mood disorders. In: Gabbard G O, ed. Treatments of psychiatric disorders. 3rd ed. Washington, D.C.: American Psychiatric Press, Inc., 2001:1307-84), but their benefit could be improved in at least two ways.

The first would be to identify pre-treatment clinical or neurobiological features that predict response versus non-response to any single treatment (predictors). Currently, ≦50% of patients reach remission following any single first-line antidepressant (Frank E. et al., Conceptualization and rationale for consensus definitions of terms in major depressive disorder. Remission, recovery, relapse and recurrence. Arch Gen Psychiatry 1991; 48:851-5; Fava M. and Davidson K. G. Definition and epidemiology of treatment-resistant depression. Psychiatr Clin North Am 1996; 19:179-200; Bileski R. J et al., A double-blind comparison of escitalopram and venlafaxine extended release in the treatment of major depressive disorder. J Clin Psychiatry 2004; 65:1190-6; Rush A. J. et al., Acute and longer-term outcomes in depressed outpatients requiring one or several treatment steps: a STAR*D report. Am J Psychiatry 2006; 163:1905-17; Trivedi M. H. et al., Evaluation of outcomes with citalopram for depression using measurement-based care in STAR*D: implications for clinical practice. Am J Psychiatry 2006; 163:28-40; Gartlehner G. et al., Comparative benefits and harms of second-generation antidepressants: Background paper for the American College of Physicians. Ann Intern Med 2008; 149:734-50), and gains in response and remission at second and subsequent steps are even more limited.

The second would be to identify features that recommend one treatment over another (so-called moderators) to find the best match between patient and treatment.

In addition to depressed mood, the diagnostic symptom criteria for Major Depressive Disorder include impaired cognitive behaviours. These behaviours encompass memory and concentration, and impaired emotional behaviours such as a bias to negative emotion (magnification of threat). Cognitive and emotional behaviours are subserved by the frontal, limbic and brainstem circuits which are also involved in the action of antidepressants, including serotonin and norephinephrine and interactions with dopamine (e.g. Austin et al., 2001).

Further, certain variations in genes impacting dopaminergic, adrenergic, serotonergic, and neurotrophic systems have been implicated in MDD and its treatment (Williams et al., 2005; and Gordon & Williams, 2010).

However, currently, there is no objective test for predicting response outcomes based on the patient's pre-treatment profile. Attempts have been made to identify clinical- or laboratory-based patient- or disease-specific characteristics to assist in treatment selection (Morishita S. and Kinoshita T. Predictors of response to sertraline in patients with major depression. Human Psychopharmacology 2008; 23:647-51; Papakostas G. I. and Fava M. Predictors, moderators and mediators (correlates) of treatment outcome in major depressive disorder. Dialogues in Clinical Neuroscience 2008; 10:439-51; Kuk A. Y et al., Recursive subsetting to identify patients in the STAR*D: A method to enhance the accuracy of early prediction of treatment outcome and to inform personalized care. J Clin Psychiatry 2010; 71:1502-8; Spronk et al. 2011; Williams et al., 2011) but current evidence is insufficient to be incorporated into guideline recommendations (Rush A. J. et al., Selecting among second-step antidepressants medication monotherapies: predictive values of clinical, demographic, or first-step treatment features. Arch Gen Psychiatry 2008; 8:870-80).

Furthermore, previous studies fail to disclose results showing which particular ADM is to be selected with confidence that administration of the selected ADM to an MDD patient will, in fact, lead to the beneficial treatment outcome. As such, due to this lack of reliable predictors of treatment outcomes, ADMs are currently still being prescribed on the basis of trial and error.

To illustrate the consequences, it is noted that in general practice a period of about 6 to 8 weeks is required to assess the effectiveness of the ADM (if any) for each individual patient and that, should the first-chosen ADM prove to have been ineffective, a further ADM will be prescribed/tried. The possibility of having to try a several ADMs over a period spanning many months before any treatment effect is observed places a great burden and stress on a patient seeking relief from the symptoms of MDD. As such, an urgent need exists to identify biomarkers which can serve as valid and reliable predictors of treatment outcomes in patients suffering from symptoms of MDD.

### SUMMARY OF THE INVENTION

It is an object of the present invention to overcome or ameliorate at least one of the disadvantages of the prior art, or to provide a useful alternative.

It is an object of the present invention to provide a test for the prediction of treatment outcomes in MDD based on the assessment of cognitive functions and/or genomic variations useful as prognostic biomarkers for MDD treatment outcomes.

The present inventors have conducted an international clinical study in which a large group of outpatients with non-psychotic MDD has been examined with a view to identifying predictors for optimised treatment in depression. The study has been designed as a real-world effectiveness trial, primarily to identify which pre-treatment characteristics could serve as the long-sought predictors or moderators of treatment response to commonly-used antidepressants or antidepressant types.

The goal of the international clinical study was to identify predictors or moderators of treatment outcomes that are sufficiently predictive to change how practitioners select among ADMs. The present inventors have found surprising, robust correlations between certain pre-treatment patient characteristics and desired treatment outcomes with respect to the three medications most commonly prescribed as first-line ADMs world-wide, namely escitalopram, sertraline and venlafaxine-extended release (venlafaxine-XR). Escitalopram and sertraline are both selective serotonin reuptake inhibitors (SSRIs) wherein venlafaxine-XR is a selective norephinephrine and serotonin reuptake inhibitor (SNRI).

The present invention relates to a method of identifying a predictor of treatment outcome in Major Depressive Disorder (MDD) comprising the steps of:
a) Determining the degree of MDD by measuring symptom scores for each member of a group of subjects with MDD
   i) before treatment with a selected antidepressant medication (ADM), and
   ii) after a predetermined period of treatment with said selected ADM,
wherein the comparison of the symptom scores measured before and after treatment with said selected ADM provides a measure of treatment outcome for each member; and
b) Assessing a cognitive and/or a genomic parameter in a group of subjects without MDD and in said group of subjects with MDD to obtain assessment scores for each parameter, wherein each member of said group of subjects with MDD is assessed
   i) before treatment with said selected ADM, and
   ii) after said predetermined period of treatment with a selected ADM; and
c) Analysing the assessment scores to establish a statistically significant correlation between
   changes in treatment outcome as indicated by changes in said symptom scores of step a) i), and a) ii), and
   changes for at least one of said parameters as indicated by changes in said assessment scores of step b) i) and b) ii),

wherein, when a change for a parameter is statistically significantly correlated with a change in treatment outcome across the group of subjects with MDD, said parameter is identified as a predictor of treatment outcome in MDD.

Generally, prior to step c) of the method of identifying a predictor of treatment outcome outlined above, the group of subjects with MDD is divided into sub-groups of MDD patients based on a comparison of:
assessment scores obtained for at least one parameter before treatment, with
assessment scores obtained for said same parameter in the group of subjects without MDD,

wherein the parameter that is statistically significantly correlated with a change in treatment outcome in step c) is a predictor of treatment outcome for subjects within one of the sub-groups of MDD patients.

In a first aspect, the present invention provides a method of predicting a treatment outcome in a patient with Major Depressive Disorder (MDD) comprising the steps of:
a) Assessing a cognitive and/or a genomic parameter in said patient thereby obtaining an assessment score for said parameter; and
b) Comparing said assessment score of step a) with a reference set of assessment scores for said parameter to establish a correlation between said assessment score with a corresponding assessment score of the reference set, wherein said corresponding assessment score is linked to a treatment outcome in MDD patients having been treated with a selected antidepressant medication (ADM),

wherein said correlation of step b) is used to predict a treatment outcome for said patient with MDD when treated with the selected ADM.

Typically, the reference set of assessment scores comprises assessment scores for cognitive and/or genomic parameters identified as statistically significant predictors of treatment outcome in MDD patients having been divided into at least two patient sub-groups based on a comparison of
assessment scores obtained by said patients for at least one parameter before treatment with a selected ADM; and
assessment scores obtained for said same parameter in a group of subjects without MDD.

Typically, the reference set of assessment scores comprises assessment scores for cognitive and/or genomic parameters identified as statistically significant predictors of treatment outcome in MDD patients having been divided into at least two patient sub-groups based on a comparison of

In some embodiments, a first cognitive parameter is a predictor of treatment outcome for MDD patients in one of the patient sub-groups when the division into said sub-groups is based on the comparison of at least a second cognitive parameter.

In alternative embodiments, a genomic parameter is a predictor of treatment outcome for MDD patients in one of the patient sub-groups when the division into said sub-groups is based on the comparison of at least one cognitive parameter.

Generally, the comparison is based on least two cognitive assessment scores and wherein the patients are divided into sub-groups of “average cognitive performance” and “below average cognitive performance”.

Treatment outcome is routinely determined by a symptom score according to the clinician-rated 17-item Hamilton Rating Scale for Depression (HRSD₁₇) or the self-rated the 16-item Quick Inventory of Depressive Symptomatology-Self Report (QIDS-SR₁₆), wherein a ≧50% decrease of a symptom score determined before treatment with a selected ADM after 8 weeks of treatment with said selected ADM indicate a treatment response and wherein symptom scores of ≦7 on the HRSD₁₇ or of ≦5 on the QIDS-SR₁₆ after 8 weeks of treatment with said selected ADM indicate remission.

In some embodiments the cognitive parameter is selected from the group consisting of motor coordination assessed by motor tapping test, decision speed assessed by choice reaction time test, verbal memory assessed by memory recall test, working memory assessed by digit span test, cognitive flexibility assessed by verbal interference test, information processing speed assessed by switching of attention test, response inhibition assessed by go/no-go test, attention assessed by continuous performance test, executive function assessed by maze test, emotion identification accuracy and speed assessed by explicit emotion test, and identification and implicit emotion bias assessed by delayed emotion recognition test.

In some embodiments the genomic parameter is selected from the group single nucleotide polymorphisms (SNPs) shown in FIG. 11.

Typically, the ADM is selected from the group consisting of selective serotonin reuptake inhibitors (SSRIs) and serotonin reuptake inhibitors (SNRIs).

In some embodiments the ADM is selected from escitalopram, sertraline and venlafaxine-extended release (venlafaxine-XR).

In some embodiments the cognitive parameter assessed is emotion identification speed and wherein the MDD patients have been divided into sub-groups based on to their assessment scores for attention or executive function, wherein either poor attention or poor executive function predicted symptom remission after 8 weeks of treatment with either escitalopram, sertraline or venlafaxine-XR.

In other embodiments the cognitive parameter assessed is cognitive flexibility and wherein the MDD patients have been divided into sub-groups based on to their assessment scores for delayed recognition of emotion speed, wherein delayed emotion identification speed predicted symptom remission after 8 weeks of treatment with either escitalopram, sertraline or venlafaxine-XR.

In a second aspect, the present invention relates to a method of predicting symptom remission or symptom response in a patient with Major Depressive Disorder (MDD) when treated with a selected ADM comprising the steps of:
a) Assessing more than one cognitive and genomic parameter in said patient thereby obtaining an assessment score for each of said parameters; and
b) Comparing said assessment scores of step a) with a reference set of assessment scores for said parameters to establish a correlation between an assessment score for at least one genomic parameter of step a) with a corresponding assessment score of the reference set,
   wherein said corresponding assessment score is linked to symptom remission or symptom response in MDD patients having been treated with a selected antidepressant medication (ADM) based on a regression model with cross-validated sensitivity or specificity of greater than 50% where said MDD patients were divided into patient cognitive performance sub-groups based a comparison of
      assessment scores obtained by said patients for at least one cognitive parameter before treatment with a selected ADM; and
      assessment scores obtained for said same cognitive parameter in a group of subjects without MDD, and
c) determining into which cognitive performance sub-group said patient falls based on said cognitive assessment scores of step a); and
d) correlating said assessment score for said at least one genomic parameter of step a) with a with a corresponding assessment score of the reference set thereby predicting symptom remission or symptom response in said patient when treated with said selected ADM.

In a third aspect, the present invention relates to a method of predicting symptom remission or symptom response in a patient with Major Depressive Disorder (MDD) when treated with a selected ADM comprising the steps of:
a) Assessing more than one cognitive parameter in said patient thereby obtaining an assessment score for each of said parameters; and
b) Comparing said assessment scores of step a) with a reference set of assessment scores for said parameters to establish a correlation between an assessment score for at least one cognitive parameter with a corresponding assessment score of the reference set,
   wherein said corresponding assessment score is linked to symptom remission or symptom response in MDD patients having been treated with a selected antidepressant medication (ADM) based on a regression model with cross-validated sensitivity or specificity of greater than 50% where said MDD patients were divided into patient cognitive performance sub-groups based a comparison of
      assessment scores obtained by said patients for at least one cognitive parameter before treatment with a selected ADM; and
      assessment scores obtained for said same cognitive parameter in a group of subjects without MDD, and
c) determining into which cognitive performance sub-group said patient falls based on said cognitive assessment scores of step a); and
d) correlating said assessment score for said at least one cognitive parameter of step a) with a with a corresponding assessment score of the reference set thereby predicting symptom remission or symptom response in said patient when treated with said selected ADM.

Typically, for methods of the present invention, the cognitive performance sub-groups are “average cognitive performance” and “below average cognitive performance” sub-groups and wherein said ADM is selected from selective serotonin reuptake inhibitors (SSRIs) and serotonin reuptake inhibitors (SNRIs).

Also, for methods of the present invention, the regression model is a univariate regression model. In some embodiments, the univariate regression model includes running separate univariate models for each of the genomic parameters, incorporating cross-validation using a k-fold approach. In some alternative embodiments, the regression model is a multivariate logistic regression model. Generally, the regression model provides a statistical significance of p<0.01.

Typically, the genomic parameters are single nucleotide polymorphisms (SNPs).

In some embodiments of the present invention, the patient falls into said “below average cognition” sub-group and wherein correlating an assessment score for MAOA rs2235186, MAOA rs979605, HTR3D rs9819507, GRIK1 rs2251036, GIRK2 rs1415485, GIRK4 rs2852217, GRIN1 rs4880213, GRIN2B rs1805490 or DARPP-32 rs907094 with a with a corresponding assessment score of the reference set predicts symptom remission in said patient when treated with escitalopram.

In other embodiments of the present invention, the patient falls into said “average cognition” sub-group and wherein correlating an assessment score for HTR2A rs2770296, GRIK2 rs2518227 or FKBP5 rs136078 with a with a corresponding assessment score of the reference set predicts symptom remission in said patient when treated with escitalopram.

In yet other embodiments of the present invention, the patient falls into said “below average cognition” sub-group and wherein correlating an assessment score for HTR2C rs540285, GRIK1 rs363478, GRIN2A rs1650397, GRIN2A rs6416623, GRIN3A rs2050641, BCL2 rs1944420, BCL2 rs2849380, FKBP5 rs1360780, NR3C2 rs1355613, or NR3C2 rs2070951 with a with a corresponding assessment score of the reference set predicts symptom remission in said patient when treated with sertraline.

In other embodiments of the present invention, the patient falls into said “average cognition” sub-group and wherein correlating an assessment score for ABCB1 rs779319 or NR3C2 rs1512343 with a with a corresponding assessment score of the reference set predicts symptom remission in said patient when treated with sertraline.

In a fourth aspect the present invention relates to a method of treating Major Depressive Disorder (MDD) in a patient, wherein said MDD is associated with a cognitive and/or a genomic parameter, said method comprising the steps of:
a) Assessing said cognitive and/or a genomic parameter in said patient thereby obtaining an assessment score for said parameter;
b) Comparing said assessment score with a reference set of assessment scores for said parameter to establish a correlation between said assessment score obtained in step a) with a corresponding assessment score of the reference set;
c) Selecting an antidepressant medication (ADM) based on said correlation of step b), and wherein said correlation is established for a parameter linked to beneficial treatment outcome in MDD patients having been treated with said ADM; and
d) Administering said ADM selected in c) to said patient to treat said MDD.

In the context of the present application the term “Major Depressive Disorder (MDD)” includes but is not limited to mood disorders such as clinical depression, major depression, unipolar depression, unipolar disorder or recurrent depression in the case of repeated episodes if diagnosed in accordance with the Diagnostic and Statistical Manual of Mental Disorders IV (DSM-IV published by the American Psychiatric Association (1994). Diagnostic and statistical manual of mental disorders (4^{th} ed). Washington D.C.). Depressive symptom severity of MDD can be rated using any appropriate rating scale including the 17-item Hamilton Rating Scale for Depression (HRSD₁₇) and the 16-item Quick Inventory of Depressive Symptomatology-Self Report (QIDS-SR₁₆).

In the context of the present application the term “biomarker” includes but is not limited to objectively measurable and assessable indicators of a biological process or biological state. Preferably, the biomarkers of the present invention indicate changes in symptom severity of MDD experienced by a patient in response to treatment. In accordance with the present invention, measurable and assessable cognitive and/or genomic parameters can be biomarkers indicating changes in symptom severity of MDD.

In the context of the present application the term “predictor of treatment outcome” includes but is not limited to biomarkers as defined above, which have a predictive quality with respect to the treatment outcome in MDD patients when treated with an antidepressant drug (ADM). “Biomarkers which have predictive quality with respect to the treatment outcome in MDD patients” here includes but is not limited to biomarkers which have been shown to be statistically significantly correlated with a change in treatment outcome in MDD patients.

In the context of the present application the term “treatment outcome” refers to certain threshold depression symptom scores measured by any appropriate rating scale, including the clinician-rated HRSD₁₇ and self-rated QIDS-SR₁₆, after treatment when compared to the symptom scores obtained before treatment. Typically, treatment outcome targets are: “symptom response”, defined for example as a ≧50% decrease from the baseline score for HRSD₁₇ or QIDS-SR₁₆; and “symptom remission”, defined for example as a score of ≦7 on the HRSD₁₇ or a score of ≦5 on the QIDS-SR₁₆.

In the context of the present application the term “statistically significant correlation” includes but is not limited to statistical correlations having p-values in a range of ≦0.05 (i.e. p-values of, ≦0.01, ≦0.005, ≦0.001, ≦0.0005, or ≦0.0001), or accuracy/sensitivity/specificity in a range of 0.50 or greater (i.e. 0.55, 0.60, 0.65, 0.70, 0.75, 0.80, 0.85, 0.90, 0.95, 0.99 or greater), depending on the specific analysis and the most relevant values for evaluating the outcome of that analysis.

In the context of the present application the term “symptom score” includes but is not limited to any objective measure of symptom severity in MDD patients. Preferably, symptom scores are determined in accordance with the Diagnostic and Statistical Manual of Mental Disorders IV (DSM-IV published by the American Psychiatric Association (1994). Diagnostic and statistical manual of mental disorders (4^{th} ed). Washington D.C.) and measured by the clinician rated HRSD₁₇ and self-rated QIDS-SR₁₆.

In the context of the present application the term “antidepressant medication (ADM)” includes but is not limited to medications which regulate the balance of effectors of mood disorders such as neurotransmitters, for example serotonin, norephinephrine or dopamine. Preferably, the ADMs are selective serotonin reuptake inhibitors (SSRIs) or selective norephinephrine and serotonin reuptake inhibitors (SNRIs) such as escitalopram or sertraline and venlafaxine, respectively.

In the context of the present application the term “cognitive parameter” includes but is not limited to parameters of general cognition and of emotional cognition. Typically such parameters are general cognitive and emotional cognitive processing skills which can be assessed by tests well-known in the art. Preferably several parameters are assessed simultaneously (Clark et al., 2006; Paul et al., 2005; Silverstein et al., 2007). A suitable battery of tests is described in Table 2 below.

In the context of the present application the term “genomic parameter” includes but is not limited to genomic variations associated with MDD. Genomic parameters can for examples be variations in genes impacting on the dopaminergic, adrenergic, serotonergic or neurotrophic systems implicated in MDD. Genomic variations can occur in the form of nucleotide insertions, substitutions or deletions within genomic DNA. Preferably the genomic parameter assessed is a single-nucleotide polymorphism (SNP) in a gene implicated in MDD. The skilled reader will appreciate that SNPs can occur in coding or non-coding regions of the genome and that each SNP is generally referred to as an allele of a genomic locus.

In the context of the present application the term “a reference set of assessment scores” includes but is not limited to a set of assessment scores for the relevant parameters previously found to be statistically significantly correlated with a symptom score of MDD thereby providing a reference set of assessment scores being statistically significant predictors of treatment outcome in MDD. The methods according to the present invention provide for the establishment of such a reference set. The reference set, which can also be an index, is useful in clinical practice to determine de novo treatment regimes with greater confidence of a beneficial treatment outcome for each individual patient as well as to determine optimized treatment regimes for patients with MDD already receiving ADM.

Reference throughout this specification to “one embodiment”, “some embodiments” or “an embodiment” means that a particular feature, structure or characteristic described in connection with the embodiment is included in at least one embodiment of the present invention. Thus, appearances of the phrases “in one embodiment”, “in some embodiments” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

### BRIEF DESCRIPTION OF THE DRAWINGS

Embodiments of the invention will now be described, by way of example only, with reference to the accompanying drawings in which:

FIG. 1 is flow diagram outlining participant recruitment, enrolment, allocation and assessment for clinical study.

FIG. 2 outlines the exclusion and Inclusion criteria for MDD participants in the clinical study.

FIGS. 3A-3C shows several bar-graphs indicating the overall remission prediction achieved by correlation of a general cognitive parameter with treatment outcome (remission) in sub-groups of patients divided based on their emotion identification speed when compared to the control group of subjects not having MDD (Method 1). Cognitive parameters: (A)—attention; (B)—executive function; and (C)—cognitive flexibility.

FIGS. 4A-4D shows several bar-graphs indicating the overall remission prediction for different treatment arms achieved by correlation of a general cognitive parameter with treatment outcome (remission) in each of the treatment arms. (A) —decision speed as predictor of remission in the escitalopram treatment arm; (B)—verbal memory as predictor of remission in the escitalopram treatment arm; (C)—poor attention skills as predictor of remission in the escitalopram and the sertraline treatment, but only for MDD patients with slow speed of emotion identification at baseline relative to this parameter in the control group of subjects not having MDD; (D) good working memory skills as predictor of remission in the sertraline and the venlafaxine-XR treatment arm, but only for MDD patients with slow speed of emotion identification at baseline relative to this parameter in the control group of subjects not having MDD.

FIG. 5 shows the stratification based on z-score variation into 2 levels of baseline cognitive performance.

FIGS. 6A-6B shows ensemble predictors of symptom remission for escitalopram in the “average cognition” sub-group (A) and in the “below average cognition” sub-group (B).

FIGS. 7A-7B shows ensemble predictors of symptom remission for sertraline in the “average cognition” sub-group (A) and in the “below average cognition” sub-group (B).

FIGS. 8A-8B shows ensemble predictors of symptom remission for venlafaxine XR in the “average cognition” sub-group (A) and in the “below average cognition” sub-group (B).

FIGS. 9 and 9A-9D are diagrams of a statistical technique used to develop a predictive model according to the present invention. Insets shown in FIGS. 9A-9D are lettered as referenced within the flowcharts shown in FIGS. 9 and 9C.

FIG. 10 shows remission rates applying the significant classifier results from the predictive model. Remission rates for all impaired performance group patients and based on predictions generated by specific classifiers, separated by treatment arm. Results with respect to (A) QIDS-SR₁₆ remission and (B) HDRS₁₇ remission using predictions from the QIDS-SR₁₆ remission sertraline classifier, and results with respect to QIDS-SR₁₆ remission using (C) predictions from the QIDS-SR₁₆ response escitalopram classifier all show differential prediction of response between sertraline and escitalopram or venlafaxine. ESC=escitalopram, SER=sertraline, VEN=venlafaxine, ‘+’=predicted to remit, ‘-’=predicted to not remit.

FIG. 11 is a list of selected SNPs considered as genomic parameters in the methods of the present invention.

FIG. 12—Results for Method 2, for outcome measures HDRS-17 response, and QIDS outcome and response.

FIG. 13—Results for Method 4, for outcome measures HDRS-17 response, and QIDS outcome and response.

FIG. 14—Results for Method 5, for outcome measures HDRS-17 response, and QIDS remission and response.

FIG. 15—Results for Method 3, for outcome measures HDRS-17 response, and QIDS remission and response.

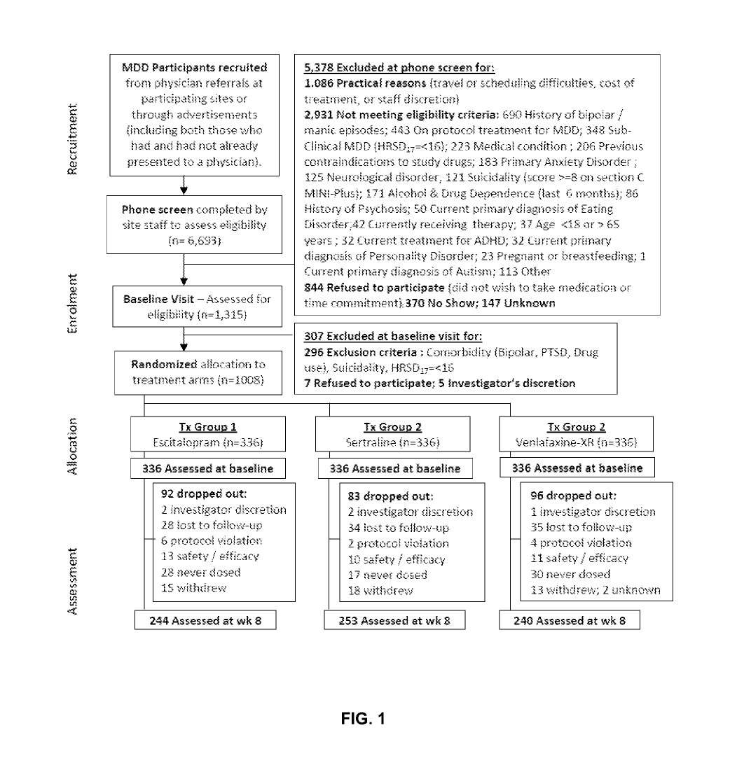

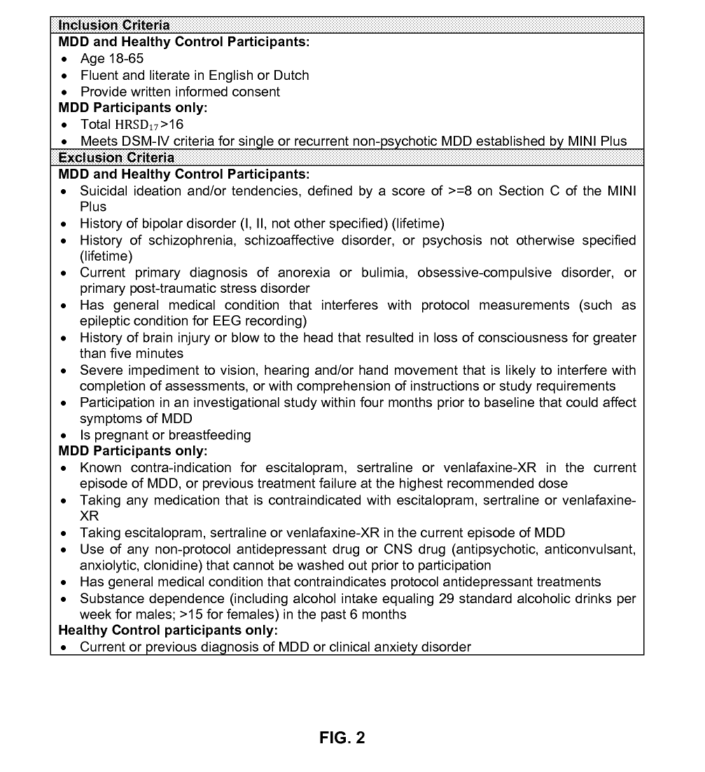

### DETAILED DESCRIPTION

Preferred embodiments of the invention will now be described.

In one preferred embodiment the present invention relates to a method of identifying a predictor of treatment outcome in Major Depressive Disorder (MDD).

At the outset, the Mini-International Neuropsychiatric Interview (MINI-Plus) (Sheehan D V, Lecrubier Y, Sheehan K H, Amorim P, Janays J, Weiller E, Hergueta T, Baker R, Dunbar G C: The Mini-International Neuropsychiatric Interview (M.I.N.I.): The development and validation of a structured diagnostic psychiatric interview for DSM-IV and ICD-10. J Clin Psychiatry 1998, 59(Suppl. 20):22-33) is used to confirm the criteria for non-psychotic MDD in accordance with the Diagnostic and Statistical Manual of Mental Disorders IV (DSM-IV published by the American Psychiatric Association 1994) and to assess for psychiatric and substance abuse disorders and other potential exclusion criteria. Depressive symptom severity can be rated using the 17-item Hamilton Rating Scale for Depression (HRSD₁₇) and the 16-item Quick Inventory of Depressive Symptomatology-Self Report (QIDS-SR₁₆). HRSD₁₇ and QIDS-SR₁₆ scores are collected at baseline (i.e. before treatment) and after a predetermined period of treatment with a selected antidepressant medication (ADM), for example after 8 weeks. In some embodiments, the QIDS-SR₁₆ is collected at baseline and after 2, 4, 6 and 8 weeks. In other embodiments, the group of subjects with MDD is assigned into separate treatment groups for treatment with a selected antidepressant medication (ADM) and the HRSD₁₇ and QIDS-SR₁₆ scores are collected again collected as described above. The HRSD₁₇ and QIDS-SR₁₆ scores provide a measure of the degree of MDD in a subject with reference to the MDD symptom severity determined. The scores obtained in the above-mentioned symptom reporting scales are referred to herein as “symptom scores”.

The comparison of the symptom scores measured before and after treatment with the selected ADM provides a measure of treatment outcome for each patient. The skilled reader will, of course, understand that such symptom scores can be measured by any applicable diagnostic method for determining the degree of MDD known in the art and that the statistical comparison of the scores can also be performed by methods commonly known in the art.

Once the baseline symptom scores have been collected, and before any treatment with an ADM has commenced, further baseline parameters are established for each of the subjects with MDD as well as for subjects of a matched control group who do not suffer from MDD. The parameters assessed in one embodiment are, for example, the subjects' general cognitive and emotional cognitive performance and/or the subjects' genomic make-up with respect to selected genomic loci. Again, the skilled addressee will appreciate that the above-described parameters are listed as examples only and that the present invention can be performed by assessing other parameters relevant to MDD.

Once all baseline measures have been recorded, the group of subjects with MDD is assigned into separate treatment arms for treatment with different, selected ADMs. Following the predetermined period of treatment with the ADM, the above-described parameters are again assessed. As indicated above, the predetermined period of treatment in one embodiment of the present invention is 2, 4, 6 and 8 weeks. By assessing the parameters before treatment with one of the selected ADMs as well as during and/or after treatment with the selected ADM, assessment scores for each individual subject in each of the treatment groups are obtained.

In some embodiments of the present invention, for the purpose of analysing the symptom scores and assessment scores obtained, subjects with MDD are divided into at least two sub-groups based on a comparison of the assessment scores obtained for at least one of said parameters in the group of subjects with MDD before treatment with the assessment scores obtained for the same parameter in the control group. For example, a division into sub-groups based on assessment scores obtained for the subjects with MDD can be based on the assessment scores of the subjects with MDD being “high” or “low”; “average”, “above average” or “below average; “similar” or “dissimilar”; “better” or “poorer”; “slower” or “faster”; etc in comparison to the assessment scores of the control group for the same parameter. The skilled addressee will appreciate that the above-mentioned division into sub-groups based on a statistical comparison of the assessment scores can be referred to as “splitting” “grouping”, “clustering”, “stratifying”, etc. the assessment scores/data obtained and that this can be performed using routine and well-known statistical methods. In some preferred embodiments the d is selected from clustering, splitting into equal proportions, or from splitting on some attribute such as a chosen z-score level or a chosen raw score value.

The assessment scores obtained are analysed to establish a statistically significant correlation between a change in treatment outcome (indicated by a relative change in symptom scores measured before, during and/or after treatment) a change for at least one of the parameters mentioned above (indicated by a relative change in assessment scores obtained before, during and/or after treatment). In one embodiment of the present invention, when a change for a parameter is statistically significantly correlated with a change in treatment outcome across one of the treatment groups, this parameter has been identified as a predictor of treatment outcome in MDD.

In some preferred embodiments the statistical method to analyse the assessment scores to establish a statistically significant correlation between a relative change in symptom scores measured before, during and/or after treatment and a relative change in assessment scores obtained before, during and/or after treatment is selected from multivariate logistic regression, univariate logistic regression and linear discrimant analysis. However, the skilled reader will understand that the analysis of the assessment scores to establish the statistically significant correlation can be performed using other routine and well-known statistical methods.

Clinical Study

In order to collect patient data with respect to the present invention, patients with MDD were recruited as potential participants in a clinical study. Of the total number of recruits, 1008 participated in the clinical study. At baseline, participant eligibility for this study and sociodemographic data was gathered. The majority of participants were women (56.6%). Mean age was 37.8±12.6 years (mean±SD). The racial distribution reflected in the patient group resembled that of the overall population of the countries in which the participating clinical centres were based (Table 1).

TABLE 1
Sociodemographic features of MDD participants at baseline

| | | | Escitalo- | | | | Venlafaxine- | |
|---|---|---|---|---|---|---|---|---|
| | Total | | pram | | Sertraline | | XR | |
| Feature | (n = 1008) | | (n = 336) | | (n = 336) | | (n = 336) | |
| | Mean | SD | Mean | SD | Mean | SD | Mean | SD |
| Age (Years) | 37.8 | 12.6 | 38.3 | 12.6 | 37.9 | 12.3 | 37.4 | 12.8 |
| Education | 14.5 | 2.8 | 14.4 | 2.9 | 14.7 | 2.7 | 14.5 | 2.8 |
| (Years) | | | | | | | | |
| | N | % | N | % | N | % | N | % |
| Gender | 571 | 56.8 | 178 | 53.0 | 191 | 56.8 | 202 | 60.1 |
| (Female) | | | | | | | | |
| Race | | | | | | | | |
| White | 625 | 62.0 | 216 | 64.3 | 205 | 61.0 | 204 | 60.7 |
| Black | 187 | 16.6 | 54 | 16.1 | 61 | 18.2 | 52 | 15.5 |
| Other | 212 | 21.0 | 65 | 19.3 | 69 | 20.5 | 78 | 23.2 |
| Unknown | 4 | 0.4 | 1 | 0.3 | 1 | 0.3 | 2 | 0.6 |
| Ethnicity | 83 | 8.2 | 27 | 8.0 | 25 | 7.4 | 31 | 9.2 |
| (Hispanic) | | | | | | | | |
| Employment | | | | | | | | |
| Employed | 506 | 50.2 | 187 | 55.7 | 157 | 46.7 | 162 | 48.2 |
| Unemployed | 66 | 6.5 | 25 | 7.4 | 20 | 6.0 | 21 | 6.2 |
| Retired | 43 | 4.3 | 10 | 3.0 | 15 | 5.7 | 14 | 4.2 |
| Student | 189 | 18.8 | 49 | 14.6 | 66 | 19.6 | 74 | 22.0 |
| Other^{a} | 73 | 7.2 | 24 | 7.1 | 29 | 8.5 | 20 | 6.0 |
| Unknown | 131 | 13.0 | 41 | 12.2 | 45 | 13.4 | 45 | 13.4 |
| Marital Status | | | | | | | | |
| Single^{b} | 614 | 60.9 | 195 | 58.0 | 198 | 58.9 | 221 | 65.8 |
| Married/ | 193 | 19.1 | 60 | 17.9 | 74 | 22.0 | 59 | 17.6 |
| cohabiting | | | | | | | | |
| Divorced/ | 142 | 14.1 | 56 | 16.7 | 49 | 14.6 | 37 | 11.0 |
| seperated | | | | | | | | |
| Widowed | 17 | 1.7 | 5 | 1.5 | 3 | 0.9 | 9 | 2.7 |
| Unknown | 42 | 4.2 | 20 | 6.0 | 12 | 3.6 | 10 | 3.0 |

Note:
All p-values were ≧0.05
^{a}Includes ‘homemaker’
^{b}Category of single includes patients who are cohabiting but identify as single, consistent with legal definition of country in which tested. Category of married/cohabiting comprises minimum estimation for cohabiting component
Abbreviations:
MDD: Major Depressive Disorder;
XR: Extended Release.

Of the total number of participants with MDD, 1008 undertook an assessment battery of general cognitive and emotional cognitive processing skills (see Table 2 below), 908 were screened for genomic variations at preselected genomic loci at baseline. Following 8 weeks of treatment with one of three commonly prescribed ADMs the general cognitive and emotional cognitive processing skills. There were assessed again. No significant differences between the three treatment arms regarding baseline sociodemographic characteristics.

TABLE 2
Cognitive assessment task names and skills assessed

| | | |
|---|---|---|
| | Cognitive | |
| Task name | Domain | Description |

General Cognition

| | | |
|---|---|---|
| Motor Tapping | Motor | Participants are required to repeatedly tap with the index finger |
| | coordination | of their dominant and then their non-dominant hand as fast as |
| | | possible for up to 60 seconds, tapping either a circle on the |
| | | screen or keyboard space bar. |
| Choice | Decision speed | Participants are required to respond to an illuminated circle as |
| Reaction Time | | fast as possible. There are 20 pseudo-random trials with a |
| | | random delay of 2 to 4 seconds following the previous |
| | | response. Participants either touch one of four circles that |
| | | lights up, or use left and right arrow keys to respond to one of |
| | | two circles that light up. |
| Memory | Verbal memory | Participants are required to recall a list of 12 words that are |
| Recall | | presented. Immediate recall is assessed over 3 to 4 |
| | | consecutive trials of presentation then recall. Delayed recall is |
| | | assessed after 20 minutes where participants are required to |
| | | recall the earlier word list without repeat presentation. |
| Digit Span | Working | Participants are required to recall in sequential order a series |
| | memory | of digits that are presented one at time on the screen, using a |
| | | 9-digit touchpad that appears after the list is presented. The |
| | | sequence length begins at 3 digits and increases progressively |
| | | throughout the task. |
| Verbal | Cognitive | Colored words with incongruent color-word combinations are |
| interference | flexibility | presented on the screen. Participants are required to firstly |
| | | identify the name (part 1) and then the color (part 2) of each |
| | | word. Assesses aspects of inhibition and interference |
| | | comparable to those indexed by the Stroop test. |
| Switching of | Information | Participants are required to identify 13 digits (1-13) and 12 |
| Attention | processing | letters (A-L) presented spatially across the screen in ascending |
| | speed | sequential order, alternating between digits and letters. |
| Go/No-Go | Response | Participants respond as quickly as possible to the word “press” |
| | inhibition | when it is presented in GREEN, and inhibit this response when |
| | | presented in RED. Participants either tap a circle on the screen |
| | | or the space bar to respond. |
| Continuous | Attention | An n-back continuous performance task in which one of 4 |
| Performance | | letters (B, C, D, G) is presented one at time in pseudo-random |
| Test | | sequence, and participants are required to respond when the |
| | | same letter appears twice in row. Participants either tap a circle |
| | | on the screen or tap the space bar to respond. |
| Maze | Executive | Participants are required to identify by trial and error a hidden |
| | function | path within a 8 × 8 grid of circles, with a total of 24 correct |
| | | moves required to move from start to finish. Task ends with two |
| | | error-free path completions, or times out after 7 minutes. |
| | | Moves are made using arrow keys on either screen or |
| | | keyboard. |

Emotional Cognition

| | | |
|---|---|---|
| Explicit | Emotion | Participants explicitly identify the emotional expression of faces |
| Emotion | identification | presented on the screen, selecting 1 of 6 word labels |
| Identification | accuracy and | presented below the face (Happy, Fear, Sad, Anger, Disgust, |
| | speed | Neutral), with 48 faces in total (8 of each emotion). |
| Delayed | Implicit emotion | Participants are presented with the same 48 faces they saw |
| Emotion | bias | previously, as well as 48 new “foil” faces, and are required to |
| Recognition | | select which of the faces they remember from the previous |
| | | task. Participants are not required to direct their attention to the |
| | | emotion displayed on the face. |

The three ADMs used in this study were escitalopram (SSRI), sertraline (SSRI) and venlafaxine-XR (SNRI). Doses for the medications were adjusted by the treating clinicians according to routine clinical practice, but following typical dose ranges: escitalopram (10 to 20 mg/day), sertraline (50 to 200 mg/day) and venlafaxine-XR (75 to 225 mg/day).

At baseline, the Mini-International Neuropsychiatric Interview (MINI-Plus) was used to confirm DSM-IV criteria for non-psychotic MDD, and assess for psychiatric and substance abuse disorders and other potential exclusion criteria. Depressive symptom severity was rated using the 17-Item Hamilton Rating Scale for Depression (HRSD₁₇) and the 16-item Quick Inventory of Depressive Symptomatology-Self Report (QIDS-SR₁₆). HRSD₁₇ was collected at baseline visit screening and week 8 visit, QIDS-SR₁₆ was collected at baseline and week 8 visits, as well as weeks 2, 4, 6. There were no significant differences between the three treatment arms regarding baseline clinical features and comorbid conditions (Table 3).

TABLE 3
Clinical features of MDD participants at baseline

| | Total | | Escitalopram | | Sertraline | | Venlafaxine-XR | |
|---|---|---|---|---|---|---|---|---|
| | (n = 1008) | | (n = 336) | | (n = 336) | | (n = 336) | |
| Feature | Mean | SD | Mean | SD | Mean | SD | Mean | SD |
| Age at first episode (years) | 22.9 | 12.0 | 23.4 | 12.6 | 23.2 | 11.9 | 22.1 | 11.5 |
| Number of episodes (*median) | 5.0* | 11.5 | 5.0* | 12.2 | 5.0* | 11.6 | 5.0* | 10.7 |
| Duration of MDD (years) | 14.4 | 12.2 | 14.2 | 12.0 | 14.2 | 12.1 | 14.7 | 12.5 |
| Depression Symptom Severity: | | | | | | | | |
| HRSD₁₇ Score | 21.9 | 4.1 | 21.8 | 4.1 | 21.9 | 4.2 | 22.0 | 4.1 |
| QIDS-SR₁₆ Score | 14.5 | 3.8 | 14.5 | 4.0 | 14.6 | 3.7 | 14.3 | 3.8 |
| | N | % | N | % | N | % | N | % |
| Family history of MDD | 232 | 23.0 | 81 | 24.1 | 77 | 22.9 | 75 | 22.3 |
| First episode ≦18 years | 478 | 47.4 | 154 | 45.8 | 154 | 45.8 | 170 | 50.6 |
| Psychiatric Comorbidity:^{a} | | | | | | | | |
| Dysthymia | 219 | 21.7 | 79 | 23.5 | 77 | 22.9 | 63 | 18.8 |
| Panic Disorder | 85 | 8.4 | 31 | 9.2 | 26 | 7.7 | 28 | 8.3 |
| Agoraphobia | 74 | 7.3 | 29 | 8.6 | 23 | 6.8 | 22 | 6.6 |
| Social Phobia | 93 | 9.2 | 31 | 9.2 | 23 | 6.8 | 39 | 11.6 |
| Specific Phobia | 55 | 5.5 | 17 | 5.1 | 21 | 6.2 | 17 | 5.1 |
| Generalized Anxiety Disorder | 69 | 6.8 | 29 | 8.6 | 17 | 5.1 | 23 | 6.8 |
| No Comorbidities | 636 | 63.1 | 200 | 59.5 | 212 | 63.1 | 224 | 66.7 |
| Previous suicide attempt | 117 | 11.6 | 38 | 11.3 | 37 | 11.0 | 42 | 12.5 |
| MDD Recurrence: | | | | | | | | |
| Recurrent MDD | 880 | 87.3 | 281 | 83.6 | 293 | 87.2 | 306 | 91.1 |
| Non-recurrent MDD | 105 | 10.4 | 43 | 12.8 | 36 | 10.7 | 26 | 7.7 |
| Unknown | 23 | 2.3 | 12 | 3.6 | 7 | 2.1 | 4 | 1.2 |
| MDD Subtypes: | | | | | | | | |
| Melancholic^{b} | 399 | 33.7 | 118 | 35.2 | 104 | 31.0 | 117 | 34.8 |
| Atypical^{c} | 442 | 43.8 | 144 | 42.9 | 145 | 43.2 | 153 | 45.5 |
| Anxious^{d} | 422 | 41.9 | 133 | 39.6 | 145 | 43.2 | 144 | 42.9 |

Note:
All p-values were ≧0.05
^{a}Refers to current comorbidities
^{b}as determined by MINI-plus and CORE
^{c}as determined by ADDS
^{d}as determined by HRSD17 criteria
Abbreviations:
HRSD₁₇: 17-item Hamilton Rating Scale for Depression (0 to 54); MDD: Major Depressive Disorder; QIDS-SR₁₆: 16-item Quick Inventory of Depressive Symptomatology-Self Report (0 to 27); XR: Extended Release

### Tests Based on Cognitive Parameters to Predict Treatment Outcomes in MDD

In one or more preferred embodiments the present invention relates to a test based on an MDD patient's performance in relation to general cognition and to emotional cognition tasks. The test is useful to predict the treatment outcome and/or response of treating MDD with a particular ADM for each individual patient and therefore is useful in assisting the practitioner to prescribe the most effective ADM at the onset of treatment.

General and Emotional Cognition Tasks

The WebNeuro assessment battery (Clark et al., 2006; Paul et al., 2005; Silverstein et al., 2007) is comprised of ten tests of traditional cognitive ability domains (general cognitive parameters)—motor tapping (assessing motor skills), choice reaction time (assessing response speed), verbal memory immediate and delayed recall, digit span (assessing working memory), stroop verbal interference (assessing cognitive control), switching of attention (assessing cognitive flexibility), go no/go test (assessing response inhibition), continuous performance test (assessing sustained attention), executive maze (assessing executive skills and planning)—and two tests of emotion processing domains (emotional cognitive parameters)—facial emotion (assessing facial emotion identification) and delayed emotion memory (assessing emotion influence on decision making). As the skilled reader will appreciate, an earlier assessment battery of tests of traditional cognitive ability domains was the IntegNeuro assessment battery. In contrast, it is noteworthy that the WebNeuro battery runs off two platforms: (1) touchscreen computer; and (2) keyboard and mouse (rather than the earlier touchscreen only IntegNeuro battery), and is accessed and completed online.

At baseline and week 8, participants completed cognitive tasks based on the above-mentioned assessment battery including nine general cognition parameters (assessing motor coordination, decision speed, memory recall, working memory, cognitive flexibility, information processing speed, response inhibition, attention, and executive function) and two emotional cognition parameters (assessing emotion identification accuracy and speed, and implicit emotion bias)—see Table 2 above. A standard, computerized touch-screen platform was used to record patient responses to each of the tasks.

Analyses were run for the entire group consisting of 1,008 MDD patients and split by treatment arm (escitalopram, sertraline, venlafaxine-XR). All analyses were conducted according to intention-to-treat principles. Data for patients who exited before week 8 (and any other missing values) were imputed using Monte-Carlo-Markov-Chain methods. Imputed values across 30 rounds were recombined to obtain an average set of outcome values. We used the relationship between acquired values for HRSD₁₇ at week 8 and the QIDS-SR₁₆ taken at weeks 2, 4 and 6 to estimate the outcome for those who left the study prior to week 8.

Treatment outcome and/or response was based on the week 8 depression symptom scores, measured by the clinician rated HRSD₁₇ and self-rated QIDS-SRᵢₛ when compared to the symptom scores obtained at baseline. Outcome targets were: “symptom response”, defined as a ≧50% decrease from the baseline score for HRSD₁₇ or QIDS-SR₁₆; and “symptom remission”, defined as a score of ≦7 on the HRSD₁₇ or a score of ≦5 on the QIDS-SR₁₆.

Predictor (Independent) Variables:

Predictor variables were comprised of the assessment scores of the 9 general cognition tasks and 2 emotional cognition tasks from the cognitive assessment battery, as listed in Table 2. Each test yielded several performance measures of accuracy and response time, which were combined to produce a single assessment score reflecting overall level of performance with respect to accuracy and response time (with exception of the Emotion Identification task which yielded 2 separate measures of accuracy and response time).

The additional factors of baseline symptom severity, site, gender and age were also considered as contributing factors. This differed between methods, and is described within the relevant section for each of the methods below. There are two main methods used: adding the additional factors into the predictive models as covariates, and regressing them out of the predictive models (when added to the prediction model, they were added as covariates, when regressing out of the prediction model, prior to building the biomarker models linear regression models were built for each possible cognition and emotion measure as the outcome and age and baseline level of severity (HDRS17) as the independent variables). Based upon the regression model, a prediction of the cognition or genomic measure was made. The difference between the actual value of the measure and the value of the measure predicted by baseline level of severity and age was used to build the reported biomarker models.

Initial preliminary analysis of the data obtained, using logistic regression to focus on general predictors across the treatment arms and specific predictors for each specific treatment arm, with results confirmed with backward conditional removal, indicated that in accordance with a method of the present invention, general prediction of remission against non-remission for subjects suffering MDD could be predicted with the following overall accuracy:

| Model | x² = 294, p < .0001, Rsq = 41% |
|---|---|
| Overall accuracy | 86.3% |
| Sensitivity | 58.4% |
| Specificity | 76.2% |

The initial model developed for prediction of remission against non remission for specific depression treatment was found to produce the following results:

| Model | X² = 361, p < .0001, Rsq = 47% |
|---|---|
| Overall accuracy | 78.5% |
| Sensitivity | 86.7% |
| Specificity | 64.3% |

This is to be compared to a randomised chance level of 50%.

The invention is further described by the following non-limiting examples.

### Method 1

One method of the present invention establishes that cognitive assessment scores can be used to predict treatment outcome with an overall accuracy of 84.7% (Sensitivity 79.0%, Specificity 90.3%), and R squared (variance explained) of 65%, using a multivariate logistic regression technique.

Design: This method was designed with the aim of establishing a highly accurate model within the 1,008 sample, by considering the interactions between general and emotional cognition values.

Sample: Data from all 1008 participants (actual and imputed data; referred to below as “patient sample”) were used in this analysis included data (actual and imputed) from all 336 participants assigned to either the escitalopram, the sertraline or the venlafaxine-XR treatment arm. No cross-validation analysis was performed.

Classification method: Multivariate logistic regression.

Co-variates: Co-varying for age and pre-treatment baseline severity.

Treatment outcomes:
i) Remission type, clinical rating (binary):
   <=7 total score on the HDRS vs. >7 total score on the HDRS₁₇
ii) Remission type, self-report rating (binary):
   <=5 total score on the QIDS-SR₁₆ vs. >5 total score on the QIDS-SR₁₆

Results: The overall model has an accuracy of 84.7% (Sensitivity 79.0%, Specificity 90.3%), and R squared (variance explained) of 65%.

### Overall Treatment Prediction in Entire Patient Sample

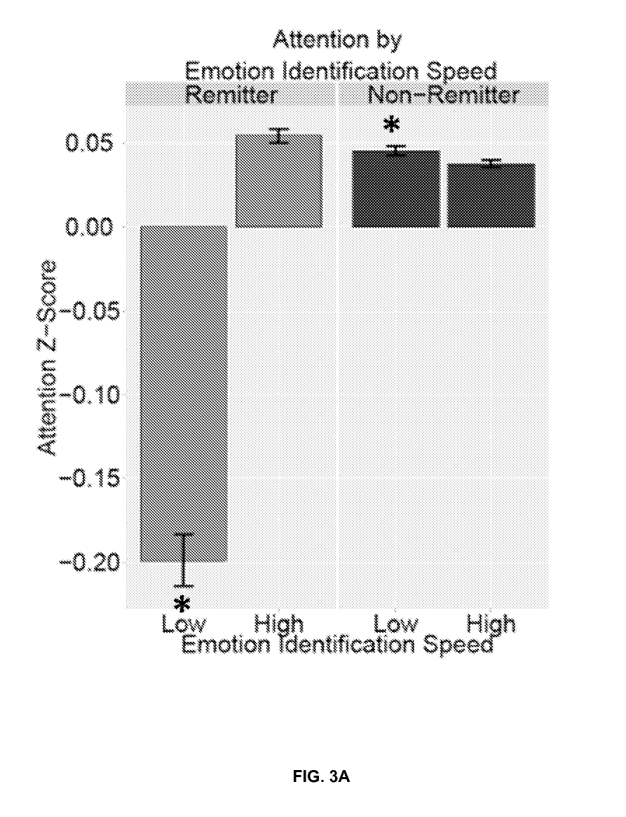

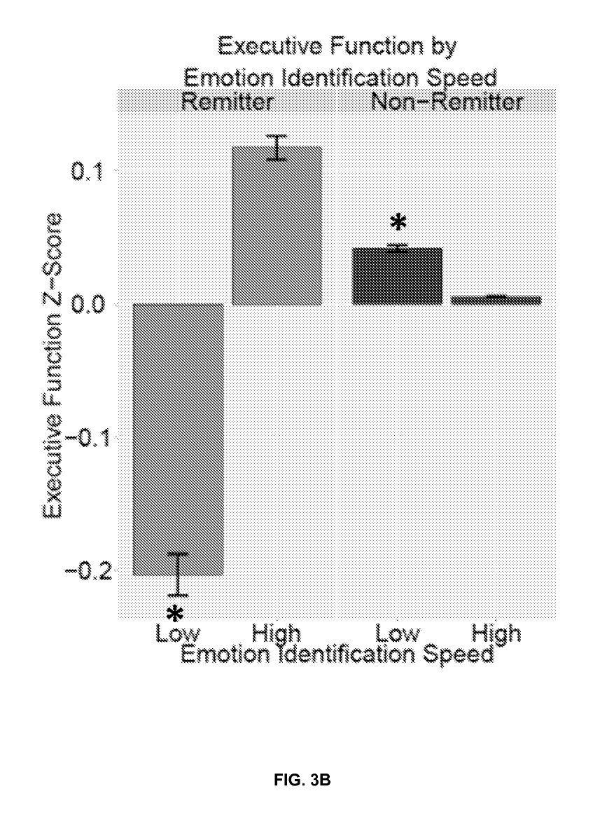

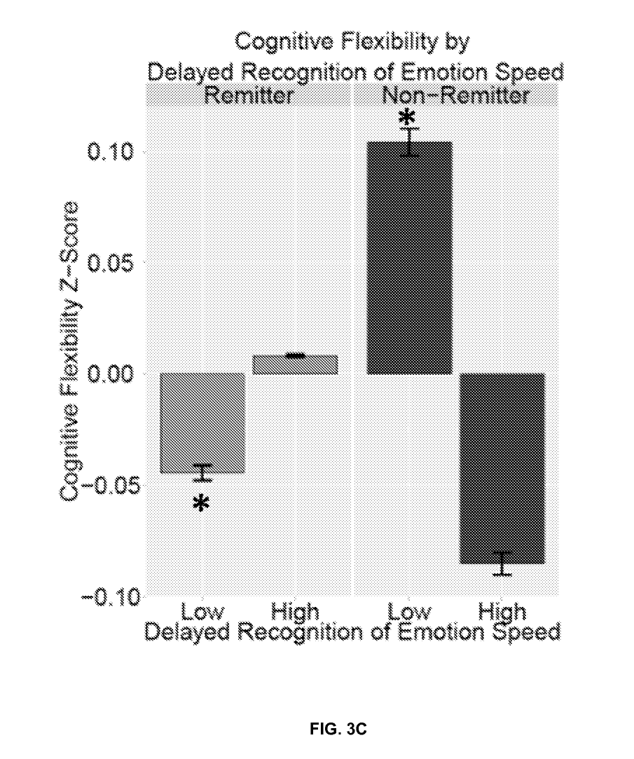

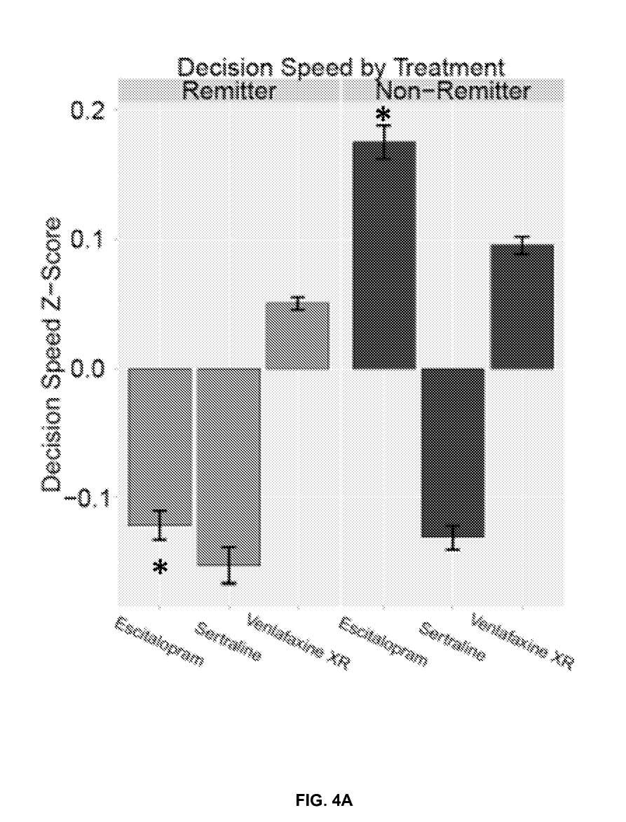

Significant predictors of treatment outcome and/or response applicable for the treatment of MDD across all treatment arms:
Interaction between Speed of Emotion Identification with Attention and with Executive Function:
   Remission is predicted by poor attention and executive function skills, and non-remission by good attention and executive function skills, but only in patients with slowed emotion identification (FIGS. 3A and B).
Interaction between Speed of Delayed Emotion Recognition and Cognitive Flexibility:
   Remission is predicted by poor cognitive flexibility, but only in patients with slowed speed for delayed emotion recognition (FIG. 3C).

### Treatment Prediction in Each Treatment Arm of 336 Samples

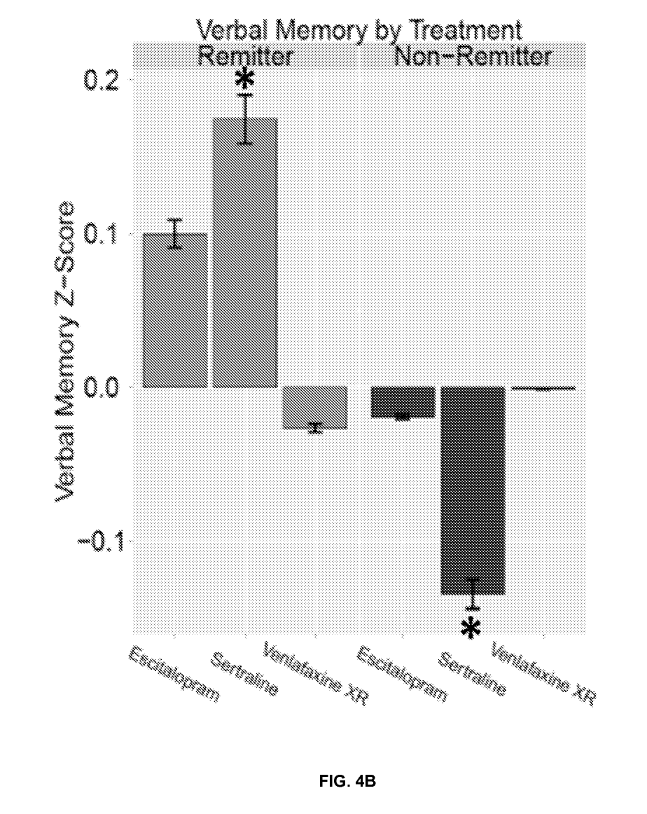

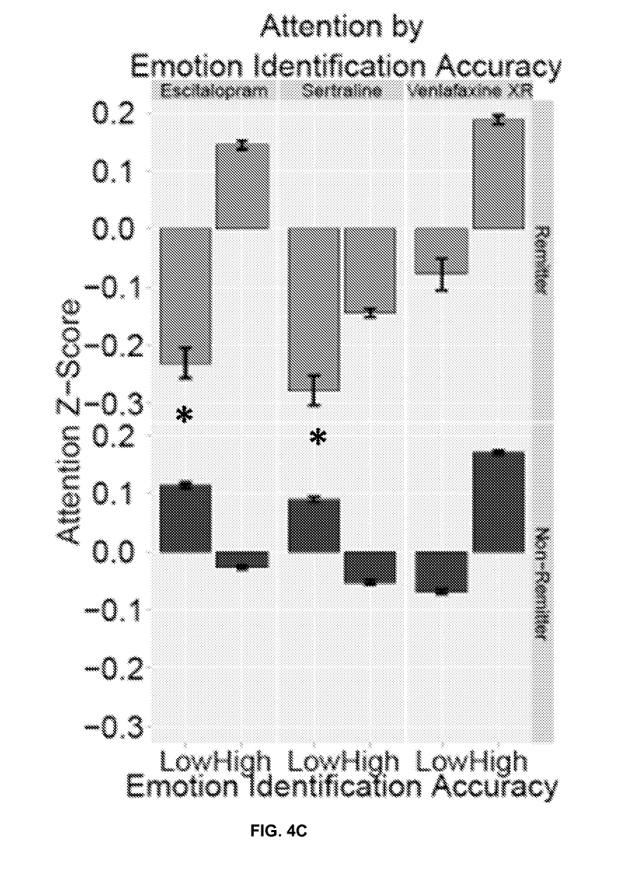

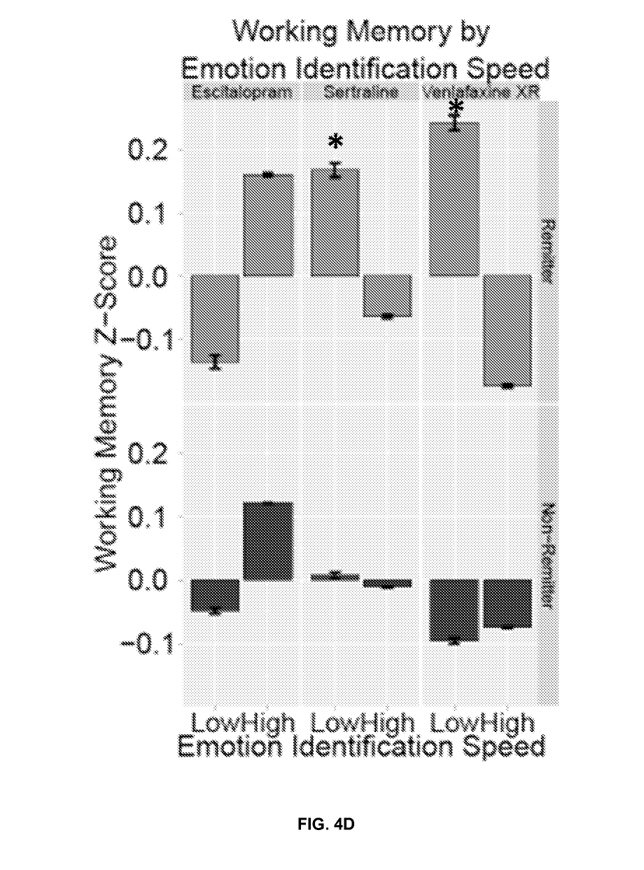

Significant predictors of treatment outcome and/or response only applicable for the treatment of MDD with escitalopram:
Decision speed constitutes main predictor of treatment outcome in patients with MDD when treated with escitalopram, wherein slower decision speed at baseline relative to this parameter in the control group of subjects not having MDD predicts remission, and wherein faster decision speed at baseline relative to this parameter in the control group of subjects not having MDD predicts non-remission (FIG. 4A) in patients with MDD.

Significant predictors of treatment outcome and/or response only applicable for the treatment of MDD with sertraline only:
Verbal memory constitutes main predictor of treatment outcome in patients with MDD when treated with sertraline, wherein better verbal memory at baseline relative to this parameter in the control group of subjects not having MDD predicts remission, and wherein poorer verbal memory at baseline relative to this parameter in the control group of subjects not having MDD predicts non-remission (FIG. 4B).

Significant predictors of treatment outcome and/or response only applicable for the treatment of MDD with escitalopram as well as sertraline but not for treatment with venlafaxine-XR:
Interaction between Speed of Emotion Identification and Attention:
   Poor attention skills predict remission in patients with MDD when treated with either escitalopram or sertraline, but only for MDD patients with slow speed of emotion identification at baseline relative to this parameter in the control group of subjects not having MDD (FIG. 4C).

Significant predictors of treatment outcome and/or response only applicable for the treatment of MDD with sertraline as well as venlafaxine-XR but not for treatment with escitalopram:
Interaction between Speed of Emotion Identification and Working Memory:
   Good working memory skills predict remission in patients with MDD when treated with either sertraline or venlafaxine-XR, but only for MDD patients with slow speed of emotion identification at baseline relative to this parameter in the control group of subjects not having MDD (FIG. 4D).

### Method 2

Another method of the present invention establishes that cognitive assessment scores can be used to predict treatment outcome with an overall cross-validated sensitivity and specificity between 54% and 65%, using a univariate logistic regression technique and an ensemble method of combining predictors with cross-validation, run in 2 separate groups defined by overall level of baseline cognitive performance.

Sensitivity/specificity values across the three medication arms range between 54% to 65%:

TABLE 4

| | Sensitivity (%) | Specificity (%) |
|---|---|---|
| | Full sample (Cross- | Full sample (Cross- |
| | validation) | validation) |
| Escitalopram | 54 (45) | 65 (56) |
| Sertraline | 65 (55) | 55 (49) |
| Venlafaxine-XR | 62 (53) | 56 (52) |

Design: This model was designed with the aim of taking three key features of the data into account which cause the results to be non-significant or over-fitted (i.e. the results were specifically tailored to the specific sample such that it did not generalize to a new sample) in other approaches such as, for example, approaches that do not use cross-validation, or that have too many variables compared to the number of subjects:
i) Heterogeneity in cognitive performance at baseline, reflecting the real world variation in the depression construct (i.e. patients with depression), is taken into account by splitting the group of MDD patients into sub-groups based on overall level cognitive performance relative to the assessment scores for this parameter in the control group of subjects not having MDD.
ii) A non-Gaussian distribution of predictive parameters is seen for the different treatment arms—for example, in some cases a low score for a particular parameter predicts treatment response in one of the treatment arms, whereas in another treatment arm a high score for this same parameter is predictive of treatment response. In this context, a simple least squares method with predictors entered into a single regression model, such as described above in Method 1, is not optimally adapted to the degree of complexity required for robust prediction of treatment outcome. As such, the present method incorporates an ensemble method known to the skilled reader of combining several predictive parameters to calculate a more reliable predictor in such cases of non-Gaussian distribution.
iii) Further, the presence of multiple predictive parameters with low degrees of significance due to multiple variables has been taken into account in the present method by limiting predictors to the top 3 contributing predictors, and by cross-validating the results obtained.

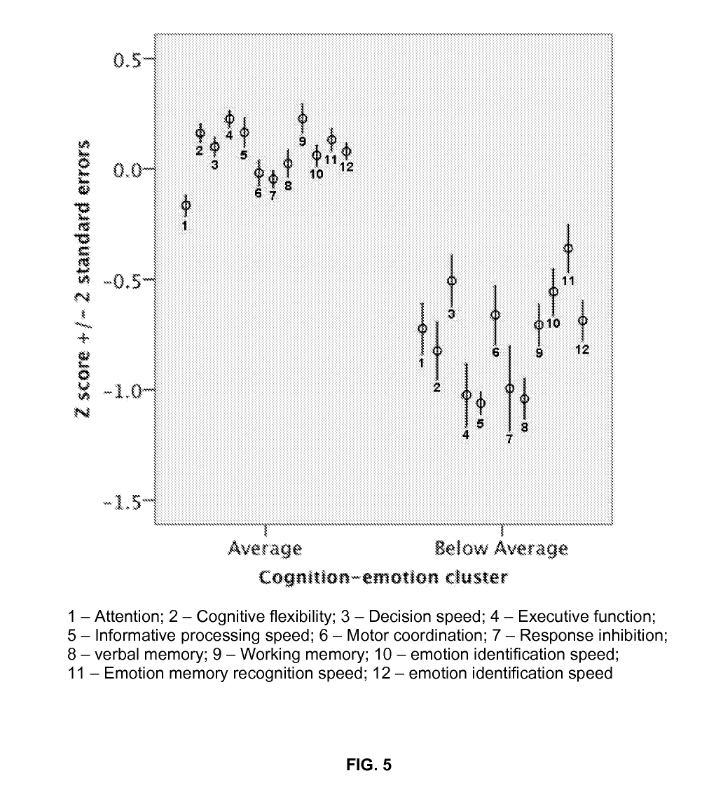

Step 1: Stratify sample at pre-treatment baseline in order to capture the heterogeneity of starting conditions. The sample was stratified according to pre-treatment cognitive and emotional performance on the subtests using a clustering technique known to the skilled reader. The sample formed two sub-groups best characterized as follows (see FIG. 5):
a) “Average cognition” sub-group (n=668 from the training sample). Defined as achieving assessment scores for most cognitive tasks which are close to the corresponding scores obtained for the control group (based on z-scores).
b) “Below average cognition” sub-group (n=340 from the training sample). Defined as achieving assessment scores for most cognitive tasks which are <=−0.5 standard deviations below the healthy peer group norm (i.e. in comparison to the control group of subjects without MDD) for three or more tasks.

All subsequent steps were run separately for these cognition level sub-groups, within each of the three treatment arms, resulting in a total of six sub-groups for which prediction modelling was performed.

Step 2: Run prediction modelling, using univariate logistic regression models (separate models for each of the 11 general and emotional cognition assessments) and incorporating cross-validation using a k-fold approach well-known in the art. Each regression was run 100 times, each time using a new random ⅘th of the sample as a “training” set, and ⅕th as a “test” set. The sample used every time was a subsample with equal numbers in each binary outcome group.

Step 3: Identify an ensemble of a small number of high-strength predictors (rather than a large number of mixed-strength predictors), identified separately for each treatment arm for each of the cognition sub-groups.
a) The three univariate models with the largest absolute beta coefficients were selected to form the ensemble to predict treatment response in each sub-group. We used simple Boolean logic to combine the top-three predictors (i.e. predictor x AND predictor y AND predictor z were required to have been identified). We then averaged the probability values for each of these top predictors. The selection of three univariate predictor models was found to provide the optimal balance of achieving high predictive accuracy without overfitting.
b) As indicated above, the ensemble technique also addresses the issue of the non-Gaussian distribution of predictive parameters, such that the small effects due to one direction (e.g. Poor performance) on one task and a different direction on another task (e.g. Good performance) were combined for optimal prediction of outcome.

Step 4: Confirm the “generalizability” of the high-strength predictors. Generalizability was quantified using a count process, such that the top predictors were required to contribute to the prediction model at least 90% of the time across 50 rounds of cross-validation in the test sub-sample. Beta coefficients were identified within the cross-validation loop for the test sample.

Step 5: Quantify the probability of predicting remission using each of the high-strength predictors selected in Step 3 and cross-validated in Step 4. To this end, the standardized beta weight for the average of the three predictors is taken for each treatment arm and for each of the cognition sub-samples.

Step 6: Provide a method of applying the prediction model for new patients in a clinical report. Probabilities of remission are obtained for each treatment arm by averaging the predictive value for the three best predictors identified. A patient is considered to be “a remitter” (i.e. being in the state of remission) if the probability of remission predicted by the model exceeds the current practice level of chance remission beyond an error margin of 5% (i.e. beyond 2.5% above or below the remission average probability based on random allocation).

Referring to Table 5, details of treatment outcome results in each of the treatment arms and cognition sub-groups are provided. Similar analysis was also performed for the non-imputed data set, and for regressing out age alone, yielding similar results in both cases.

Across all treatment arms, remission was predicted by different profiles of cognitive performance for the “average” and “below average” cognition sub-groups, and better prediction outcomes were demonstrated than for the data set without the division into sub-groups.

All Treatment Arms Combined—

Across treatment arms, remission was predicted with 63% sensitivity and 52% specificity (with cross-validation 59% sensitivity and 51% specificity). For the “average cognition” sub-group, the presence of better attention and emotion identification skills in combination with faster decision speed predicted better probability of remission. For the “below average cognition” sub-group, the combination of better verbal memory, emotion identification and information processing skills predicted better probability of remission. However, remission outcomes for each of the treatment arms were predicted by combinations of cognitive and emotional function distinct from these present for the entire group of 1008 participants.

Escitalopram—

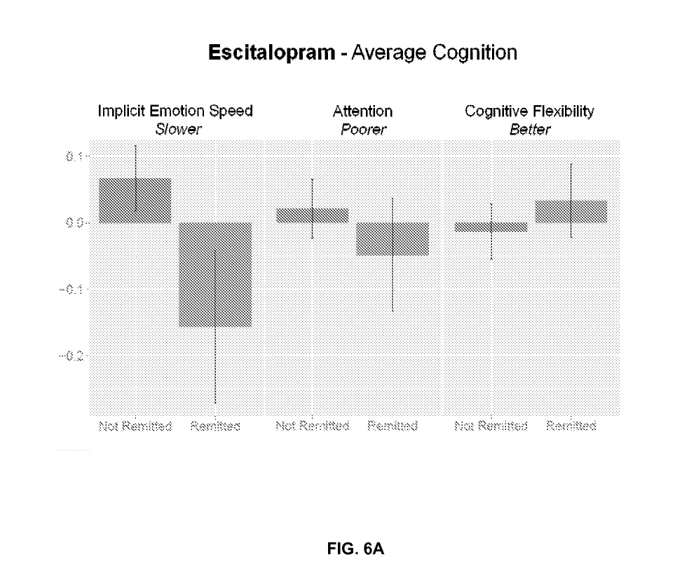

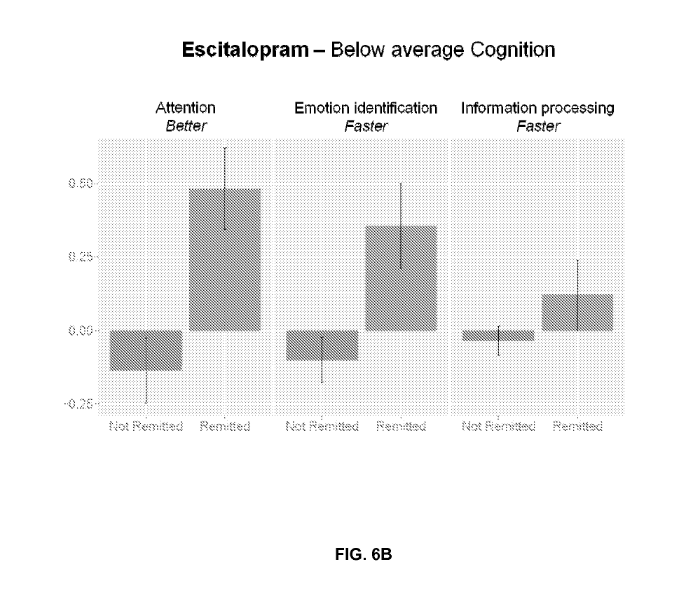

For escitalopram, remission was predicted with 54% sensitivity and 65% specificity (with cross-validation 45% sensitivity and 56% specificity). The combination of slower implicit emotion speed, poorer attention skills, but better cognitive flexibility, provided the best prediction of remission for the “average cognition” group. The combination of better attention skills with faster information processing and emotion identification speed provided the best prediction of symptom remission for the “below average cognition” group (see FIG. 6).

Sertraline—

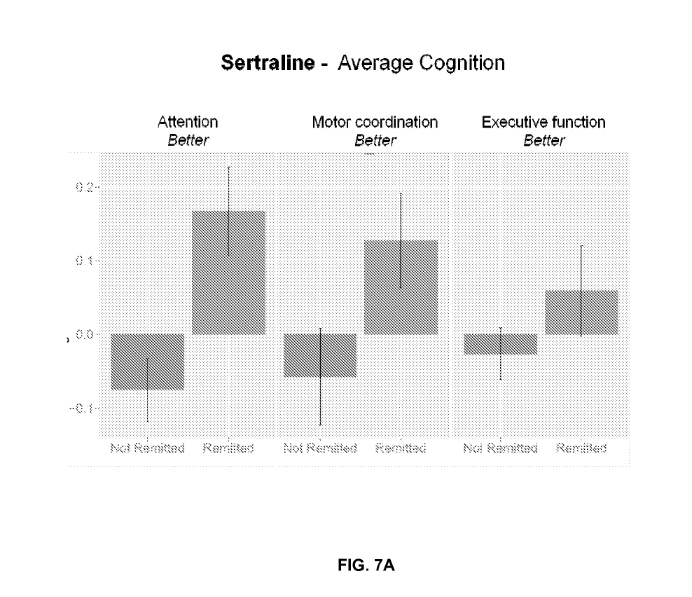

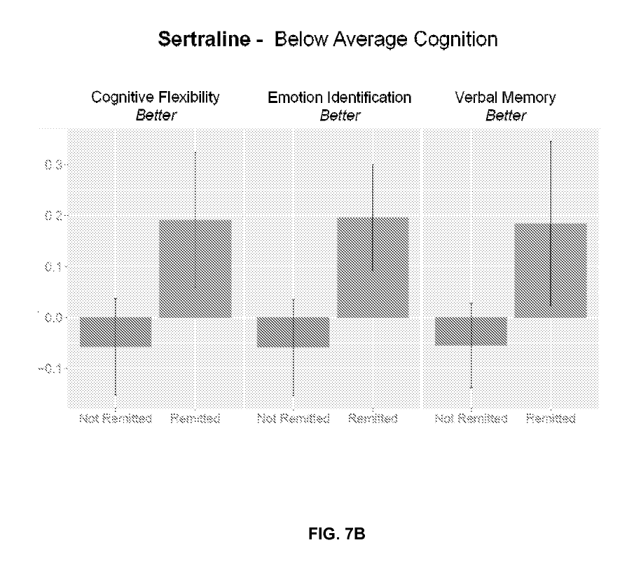

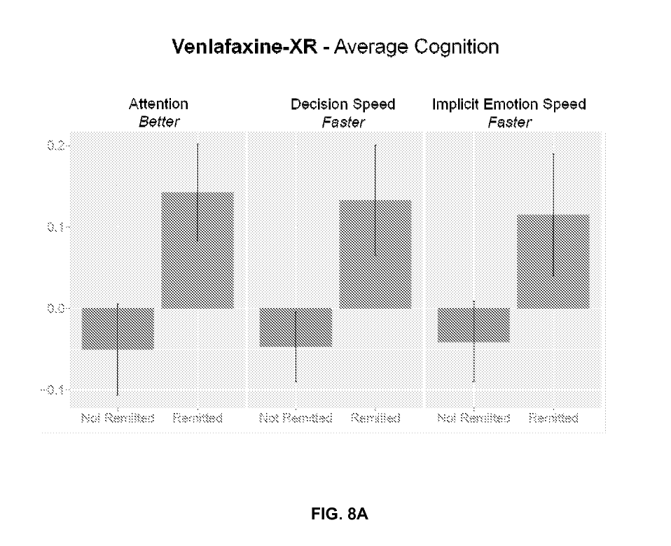

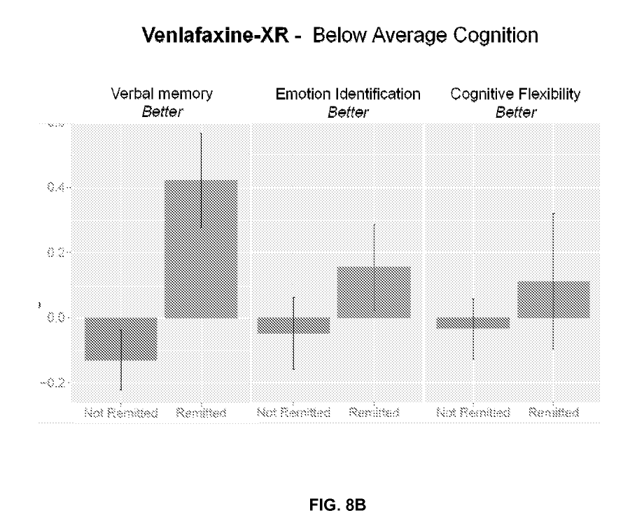

For sertraline, remission was predicted with 65% sensitivity and 55% specificity (with cross-validation 55% sensitivity and 49% specificity). For the “average cognition” sub-group, the combination of better attention, motor coordination and executive function skills best predicted remission. For the “below average cognition” sub-group it was a combination of better cognitive flexibility, verbal memory and emotion identification skills that best predicted remission (see FIG. 7).

TABLE 5
Outcome results for the final prediction model. The “total” value for each treatment arm
(and the combined treatment arm) are the values for the full group (336 for each treatment arm,
1008 for the combined treatment arms) before they are split by cognition groups.

| | | | | | | | |
|---|---|---|---|---|---|---|---|
| | | | Positive | | Negative | Predictors | |
| | | | Predictive | Speci- | Predictive | (in order of statistical | |
| Group | Model | Sensitivity | Power | ficity | Power | contribution) | Beta |

All treatment arms combined

| | | | | | | | |
|---|---|---|---|---|---|---|---|
| Total | All patients | 0.67. | 0.47 | 0.32 | 0.79 | | |
| | Cross | 0.66 | 0.32 | 0.46 | 0.78 | | |
| | validation | | | | | | |
| Average | All patients | .63 | .35 | .50 | .76 | Attention | .38 |
| cognition | Cross | .58 | .32 | .48 | .73 | Decision Speed | .32 |
| | validation | | | | | Emotion Identification | .21 |
| Below | All patients | .58 | .28 | .56 | .81 | Verbal Memory | .52 |
| average | Cross | .54 | .25 | .52 | .80 | Emotion Identification | .32 |
| cognition | validation | | | | | Information Processing | .28 |

Escitalopram

| | | | | | | | |
|---|---|---|---|---|---|---|---|
| Total | All patients | 0.63 | 0.47 | 0.32 | 0.76 | | |
| | Cross | 0.57 | 0.30 | 0.45 | 0.72 | | |
| | validation | | | | | | |
| Average | All patients | .42 | .36 | .66 | .72 | Implicit Emotion Speed | −.39 |
| cognition | Cross | .35 | .25 | .54 | .65 | Attention | −.18 |
| | validation | | | | | Cognitive Flexibility | .15 |
| Below | All patients | .81 | .39 | .60 | .91 | Emotion Identification | 1.14 |
| average | Cross | .74 | .35 | .60 | .89 | Speed Attention | 1.03 |
| cognition | validation | | | | | Information Processing | .73 |

Sertraline

| | | | | | | | |
|---|---|---|---|---|---|---|---|
| Total | All patients | 0.63 | 0.47 | 0.32 | 0.76 | | |
| | Cross | 0.57 | 0.30 | 0.45 | 0.72 | | |
| | validation | | | | | | |
| Average | All patients | .69 | .40 | .53 | .78 | Attention | .96 |
| cognition | Cross | .61 | .39 | .54 | .74 | Motor coordination | .42 |
| | validation | | | | | Executive Function | .40 |
| Below | All patients | .62 | .25 | .45 | .80 | Cognitive flexibility | .43 |
| average | Cross | .39 | .18 | .44 | .71 | Emotion identification | .42 |
| cognition | validation | | | | | Verbal memory | .39 |

Venlafaxine-XR

| | | | | | | | |
|---|---|---|---|---|---|---|---|
| Total | All patients | 0.63 | 0.47 | 0.32 | 0.76 | | |
| | Cross | 0.57 | 0.30 | 0.45 | 0.72 | | |
| | validation | | | | | | |
| Average | All patients | .65 | .33 | .51 | .80 | Decision speed | .68 |
| cognition | Cross | .54 | .27 | .49 | .75 | Attention | .50 |
| | validation | | | | | Implicit emotion speed | .40 |
| Below | All patients | .62 | .31 | .59 | .84 | Verbal Memory | .88 |
| average | Cross | .55 | .27 | .54 | .80 | Cognitive flexibility | .27 |
| cognition | validation | | | | | Emotion identification | .19 |
| Below | All patients | .62 | .25 | .45 | .80 | Cognitive flexibility | .43 |
| average | Cross | .39 | .18 | .44 | .71 | Emotion identification | .42 |
| cognition | validation | | | | | Verbal memory | .39 |

Venlafaxine-XR

| | | | | | | | |
|---|---|---|---|---|---|---|---|
| Total | All patients | .62 | .32 | .56 | .81 | | |
| | Cross | .53 | .27 | .52 | .76 | | |
| | validation | | | | | | |
| Average | All patients | .65 | .33 | .51 | .80 | Decision speed | .68 |
| cognition | Cross | .54 | .27 | .49 | .75 | Attention | .50 |
| | validation | | | | | Implicit emotion speed | .40 |
| Below | All patients | .62 | .31 | .59 | .84 | Verbal Memory | .88 |
| average | Cross | .55 | .27 | .54 | .80 | Cognitive flexibility | .27 |
| cognition | validation | | | | | Emotion identification | .19 |

Venlafaxine-XR—

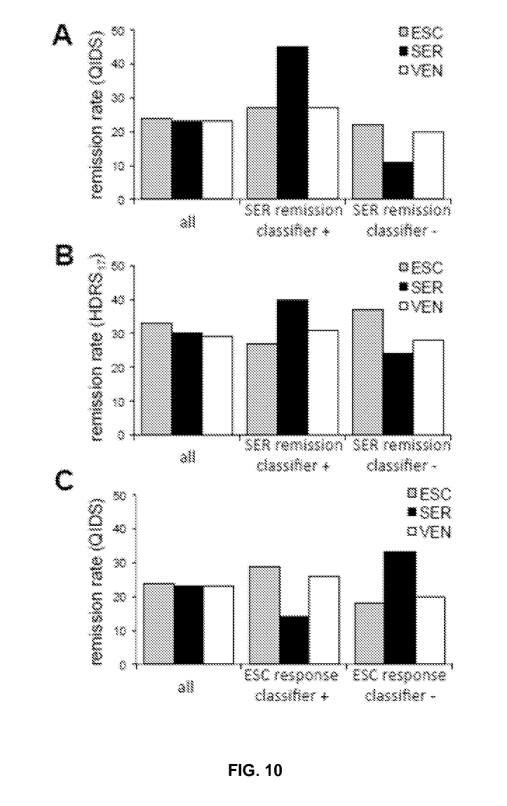

For venlafaxine-XR, remission was predicted with 62% sensitivity and 56% specificity (with cross-validation 53% sensitivity and 52% specificity). For the “average cognition” sub-group, the combination of better attention skills with faster decision speed and implicit emotion speed best predicted remission. For the “below average cognition” sub-group, similar to sertraline, remission was best predicted by a combination a combination of better cognitive flexibility, verbal memory and emotion identification skills (see FIG. 10).

The predictive model strongest in negative predictive power, ranging between 79% and 81% across treatment arms (with cross-validation 71% to 76%). Positive predictive power is weaker overall, ranging between 32% to 36% across treatment arms (with cross-validation 27% to 31%). For specific treatment arm by cognition group subsets, sensitivity is also low, indicating that the test is performing at the same level as chance for these specific groups. For the prediction of remission with escitalopram for the average cognition group, sensitivity is 42% (35% with cross-validation). The prediction of remission with sertraline is good for the full sample at 62%, but lower with cross-validation at 39%.

Across each of the 3 treatment arms, the predictive model performs with 54% to 65% sensitivity and 55% to 65% specificity (with cross-validation 45% to 55% sensitivity and 49% to 56% specificity).

### Method 3

A further method of the present invention establishes that cognitive assessment scores can be used to predict treatment outcome with an overall accuracy of up to 75% for some treatment arms using a linear discriminant analysis technique known in the art with (a) an ensemble classifier used to perform the classifications, and (b) cross-validation used as an inherent step within the model development to include only highly reliable results within the developed model, run in 2 separate groups defined by overall level of baseline cognitive performance.

Design: This method was designed with the aim of identifying the highly reliable results within the development of the model itself, by including cross-validation methods within the development of the model itself. This model was also designed with the aim of minimizing the role of manual and arbitrary decisions in the development of the model, by using a pattern classification technique of linear discriminant analysis to develop the model parameters.

First, the sample was stratified as described at [0116] above in Step 1 of Method 2 (see FIG. 5). Again, all subsequent steps were run separately for these cognition level sub-groups, within each of the three treatment arms, resulting in a total of six sub-groups for which prediction modelling was performed.

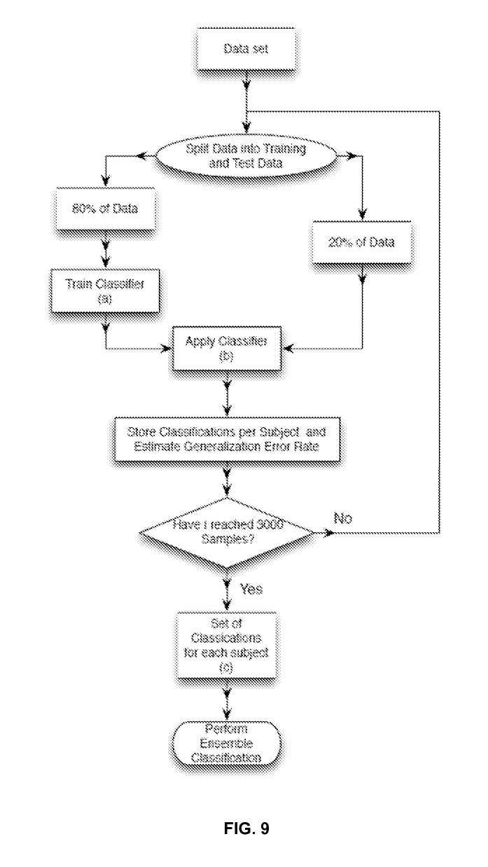

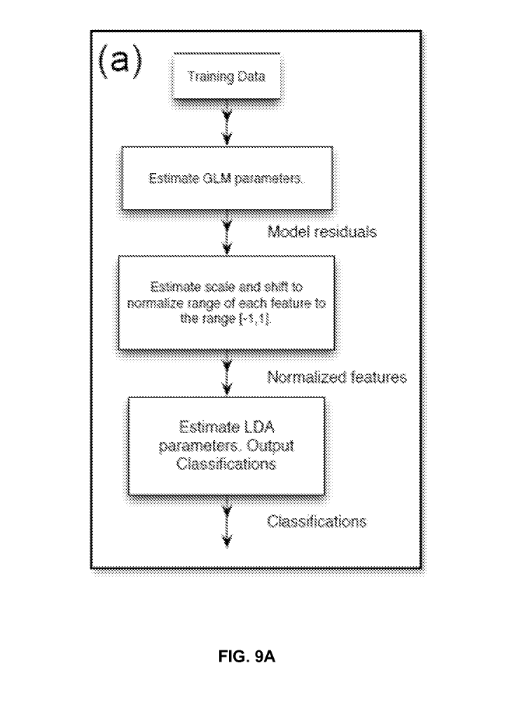

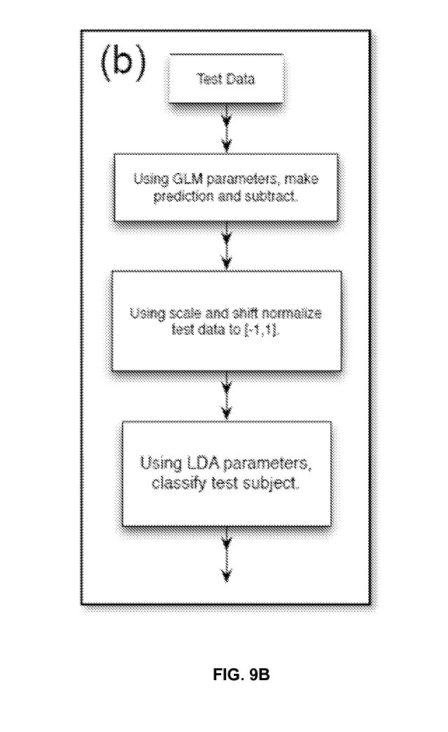

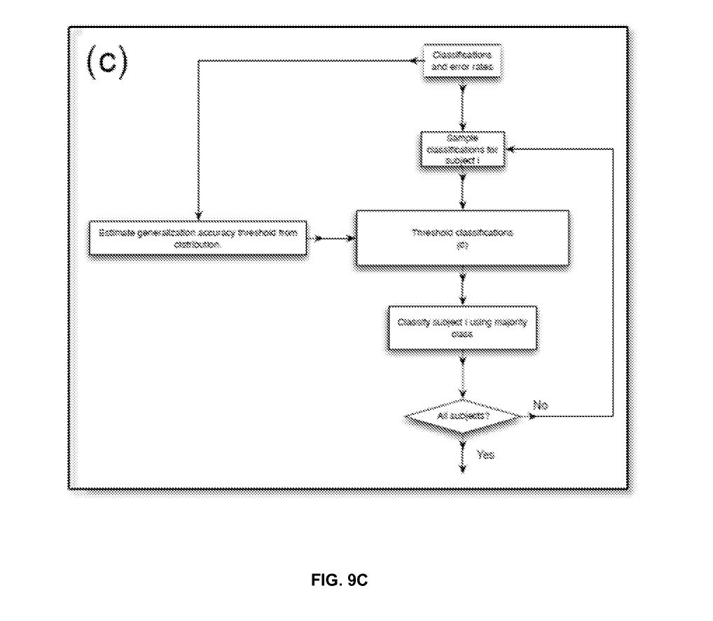

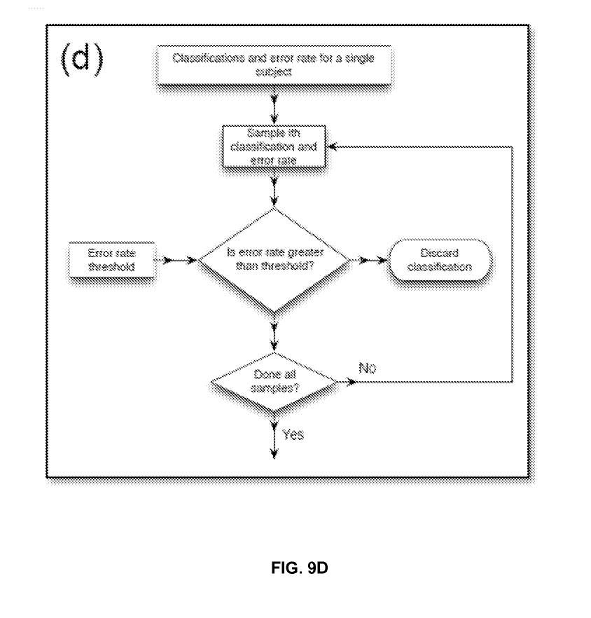

An ensemble classifier was used to perform classifications where cross-validation was used to determine the classification error rate (see FIG. 9). For each classification, 3000 bootstrap sub-samples were taken from the original data set to train models and apply them. Each bootstrap sub-sample was comprised of 80% of the full sample, further limited by the size of the smaller treatment outcome group, with equal number of samples from each treatment outcome group (e.g. if a sample contained 80 remitters and 100 non-remitters, a bootstrap sub-sample would contain a random set of 64 remitters and 64 non-remitters). As such, each bootstrap sub-sample constituted a classification model.

The generalization accuracy of each sub-sample classification model was determined using an internal leave-one-out cross-validation approach known in the art over 50 further sub-samplings for each bootstrap sub-sample. Each of these internal classification models was comprised of three major steps:
1.) the removal linear trends related to age, gender, education, study site and severity (on the target measure for classification),
2.) normalization of the feature range to within a [−1,1] range, and
3.) linear discriminant analysis (LDA).

Model parameters for each of the blocks were estimated only from within the training set and excluded subjects left out for cross-validation. By determining the generalization accuracy of each of the 3000 bootstrap sub-sample models, we could select optimal models to test on the sample left out of the original bootstrap sub-sampling. This model generalization accuracy selection threshold was identified in an unbiased manner by fitting a mixture of two Gaussian distributions to accommodate well and poorly generalizing models. The final classification for each subject was determined using a majority vote from the retained classifications. The significance of each final classifier was determined by permuting remitter/non-remitter labels for each subject (one-sided p-value).

Results: The best results for predicting symptom remission were found for sertraline for the “below average” cognition sub-group, with an accuracy of 66% for self-reported and 74% for clinician-reported scores of symptom severity (sensitivity of 69% and 59%, and specificity of 59% and 69%, respectively).

The best result for predicting symptom response was found for escitalopram for the “below average” cognition sub-group for the self-reported scores of symptom severity, with 61% accuracy (sensitivity 68% and specificity 58%). No significant results were found for the “average” cognition sub-group, or for prediction of symptom remission or response to venlafaxine-XR (see Table 6 and FIGS. 9 and 10).

TABLE 6
Treatment outcome results for the prediction model according to Method 3.

| | performance | | | | | | |
|---|---|---|---|---|---|---|---|
| | cluster | medication | accuracy | sensitivity | specificity | PPV | NPV |
| Remission | | | | | | | |
| criterion | | | | | | | |
| QIDS-SR16 | all subjects | ESC | 48 | 49 | 48 | 27 | 70 |
| remission | (N = 1008) | SER | 54 | 61 | 51 | 33 | 77 |
| | | VEN | 46 | 51 | 44 | 24 | 72 |
| | intact | ESC | 40 | 35 | 43 | 21 | 59 |
| | performance | SER | 55 | 59 | 54 | 37 | 74 |
| | (N = 668) | VEN | 53 | 58 | 51 | 30 | 77 |
| | impaired | ESC | 52 | 56 | 51 | 26 | 79 |
| | performance | SER | 74 | 69 | 75 | 45 | 89 |
| | (N = 340) | VEN | 45 | 35 | 48 | 17 | 71 |
| HRSD17 | all subjects | ESC | 49 | 52 | 48 | 29 | 72 |
| remission | (N = 1008) | SER | 54 | 63 | 51 | 34 | 77 |
| | | VEN | 44 | 53 | 40 | 24 | 72 |
| | intact | ESC | 49 | 51 | 48 | 41 | 58 |
| | perfomance | SER | 49 | 48 | 49 | 41 | 56 |
| | (N = 668) | VEN | 52 | 57 | 49 | 37 | 68 |
| | impaired | ESC | 58 | 54 | 59 | 39 | 73 |
| | performance | SER | 66 | 59 | 69 | 44 | 80 |
| | (N = 340) | VEN | 49 | 48 | 49 | 28 | 70 |
| Response | | | | | | | |
| criterion | | | | | | | |
| QIDS-SR16 | all subjects | ESC | 55 | 56 | 55 | 43 | 67 |
| response | (N = 1008) | SER | 56 | 59 | 53 | 45 | 67 |
| | | VEN | 45 | 50 | 42 | 33 | 60 |
| | intact | ESC | 49 | 49 | 49 | 38 | 60 |
| | performance | SER | 57 | 59 | 56 | 52 | 63 |
| | (N = 668) | VEN | 49 | 53 | 47 | 37 | 63 |
| | impaired | ESC | 61 | 68 | 58 | 47 | 76 |
| | performance | SER | 41 | 45 | 40 | 23 | 64 |
| | (N = 340) | VEN | 49 | 45 | 51 | 31 | 64 |
| HRSD17 | all subjects | ESC | 56 | 58 | 52 | 66 | 44 |
| response | (N = 1008) | SER | 53 | 57 | 46 | 68 | 34 |
| | | VEN | 44 | 40 | 52 | 62 | 31 |
| | intact | ESC | 51 | 51 | 49 | 65 | 36 |
| | perfomance | SER | 53 | 53 | 54 | 77 | 28 |
| | (N = 668) | VEN | 43 | 42 | 45 | 61 | 28 |
| | impaired | ESC | 48 | 44 | 53 | 55 | 42 |
| | performance | SER | 47 | 49 | 45 | 49 | 45 |
| | (N = 340) | VEN | 50 | 49 | 50 | 64 | 35 |

### Method 4:

According to yet a further method of the present invention cognitive assessment scores can be used to predict treatment outcome with an overall cross-validated sensitivity and specificity between 57% and 64%, using a multivariate logistic regression technique, with predictors grouped into separate regression models for general cognition and emotional cognition parameters serving as predictors as well as parameters for the division of the group of subjects with MDD into sub-groups based on a comparison of the assessment scores obtained the respective parameters at baseline with the assessment scores obtained for the corresponding parameters in the group of subjects without MDD. Dividing the data obtained from the group of subjects with MDD into sub-groups based on z-score cut-offs for the respective parameters.

Design: This method was designed with the aim of identifying the most accurate predictions that could be achieved across the maximal number of patients and drug types, by exploring different methods of sub-typing patients based on level of performance on specific cognitive tasks (1 single performance score for each task), and different methods of entering the cognition predictors (single task scores grouped by cognition and emotion categories).

Step 1: Stratify sample at pre-treatment baseline in order to capture the heterogeneity of starting conditions. The sample was stratified according to pre-treatment cognitive and emotional performance on the subtests, using a series of different measures and splitting points to determine the optimal stratification for treatment prediction utility. Two types of baseline splitting of data was explored, splitting into 2 groups (around a z-score cut-off level, with different z score levels considered and tested in 0.5 increments from −3 to +3), and splitting into 3 equal groups of the top, middle, and lowest ⅓ of the group. Both of these approaches were run on the full averaged cognitive performance score, and for each individual task, yielding 13 different scores that were tested with each approach. These splits were assessed based on providing the highest cross-validated accuracy, while applying to the most number of patients and the greatest number of treatment arms.

Step 2: Run prediction modelling, using multivariate logistic regression models, for the full group and each treatment arm, for each splitting approach as described in Step 1 In order to minimize type 1 error rates, only results yielding cross-validated sensitivity or specificity values that were above 0.60 (with the other above 0.55) were considered for evaluation. All regressions were developed on the full sample then cross-validated 50 times using 80% as training set and 20% as testing set.

Predictors were comprised of a single overall performance score for each task, and grouped according to cognition and emotion task measures, with separate predictive models run for main effects alone and for main and interaction effects together, for cognition and emotion task measures run separately and in combination, yielding a total of 6 models for each analysis. This approach was selected due to the large number of predictors, to maximize power to detect small effects. Only the model with the strongest cross-validated results in each case was reported for consideration in the final results.

All results are presented for prediction of symptom remission based on the clinician rated HDRS-17.

Results: For escitalopram, the optimal predictive results were achieved by splitting the group into 2 sub-groups based on a combined score across all of the cognitive tasks, at a z-score level of −0.5. For the Low cognitive performing group (z score<−0.5), the best predictor of remission was the cognition main effect model, yielding a cross-validated accuracy of 60% (sensitivity 60%, specificity 60%). For the High cognitive performing group, the best predictor of remission was the combined cognition and emotion main effect model, yielding a cross-validated accuracy of 57% (sensitivity 61%, specificity 55%). In combination, these two predictive models for the high and low cognitive performing groups provide a predictive model for escitalopram for 100% of the sample at 57% to 60% accuracy.

TABLE 7
Treatment outcome results for the prediction model according to Method 4.
Sample split into 2 subgroups by overall cognitive performance at z = −0.5

| | | | | |
|---|---|---|---|---|
| Subgroup | | | | |
| split | Accuracy | Sensitivity | Specificity | Predictors |

Full 1008 sample

| | | | | |
|---|---|---|---|---|
| Low | 0.54 | 0.52 | 0.55 | Cognition main effects |
| High | 0.53 | 0.52 | 0.53 | Cognition main effects |

Escitalopram

| | | | | |
|---|---|---|---|---|
| Low | 0.60 | 0.60 | 0.60 | Cognition main effects |
| High | 0.57 | 0.61 | 0.55 | Cognition and Emotion main effects |

Sertraline

| | | | | |
|---|---|---|---|---|
| Low | 0.55 | 0.49 | 0.58 | Cognition and Emotion main and |
| | | | | interaction effects |
| High | 0.55 | 0.48 | 0.60 | Cognition main and interaction effects |

Venlafaxine-XR

| | | | | |
|---|---|---|---|---|
| Low | 0.51 | 0.58 | 0.47 | Emotion main and interaction effects |
| High | 0.54 | 0.47 | 0.59 | Cognition and Emotion main and |
| | | | | interaction effects |

Sample split into 3 subgroups by Response Inhibition performance, at
equal 1/3 low, middle and high performance groups
Full 1008 sample

| | | | | |
|---|---|---|---|---|
| Low | 0.51 | 0.58 | 0.47 | Emotion main and interaction effects |
| Middle | 0.54 | 0.47 | 0.59 | Cognition and Emotion main and |
| | | | | interaction effects |
| High | 0.51 | 0.58 | 0.47 | Emotion main and interaction effects |

Escitalopram

| | | | | |
|---|---|---|---|---|
| Low | 0.53 | 0.45 | 0.57 | Cognition main effects |
| Middle | 0.56 | 0.64 | 0.49 | Emotion main effects |
| High | 0.62 | 0.50 | 0.68 | Emotion main effects |

Sertraline

| | | | | |
|---|---|---|---|---|
| Low | 0.53 | 0.45 | 0.57 | Cognition main effects |
| Middle | 0.56 | 0.64 | 0.49 | Emotion main effects |
| High | 0.62 | 0.50 | 0.68 | Emotion main effects |

Venlafaxine-XR

| | | | | |
|---|---|---|---|---|
| Low | 0.53 | 0.58 | 0.50 | Emotion main effects |
| Middle | 0.49 | 0.34 | 0.57 | Cognition main and interaction effects |
| High | 0.60 | 0.65 | 0.59 | Emotion main and interaction effects |

For sertraline and venlafaxine-XR, the best, the optimal predictive results were achieved by splitting the group into 3 equal sub-groups based on the total task score for Response Inhibition, reflecting low, middle and high performing sub-groups. Predictive models with sufficient accuracy were established for the middle and high performing groups for sertraline (66% of the sample), and for the high performing group alone for venlafaxine-XR (33% of the sample).

For sertraline, the best predictors of remission were the combined cognition and emotion main effect models for both the middle and high performing groups, yielding cross-validated accuracies of 60% for the middle performing group (sensitivity 61%, specificity 58%) and 64% for the high performing group (sensitivity 64%, specificity 63%). For venlafaxine-XR, the best predictor model for remission for the high performing group was the combined emotion main and interaction effect model, yielding a cross-validated accuracy of 60% (sensitivity 65%, specificity 59%).

### Method 5

In accordance with this further method of the invention, cognitive assessment scores can be used to predict treatment outcome with an overall cross-validated sensitivity and specificity between 57% and 64%, using a multivariate logistic regression technique, with predictors grouped into separate regression models for cognition and emotion main and interaction effects, and splitting of the overall sample into sub-groups based on z-score cut-offs for performance on specific cognitive task baseline performance.

Design: This method was designed with the aim of identifying the most accurate predictions that could be achieved across the maximal number of patients and drug types, by exploring different methods of sub-typing patients based on level of performance on specific cognitive tasks (2 scores for each task—average performance and consistency of performance), and different methods of entering the cognition predictors (individual task measure scores grouped by cognition and emotion categories, and the organization of the measure scores within each category determined using 5 factor principle components analysis).

Step 1: Stratify sample at pre-treatment baseline in order to capture the heterogeneity of starting conditions. The sample was stratified according to pre-treatment cognitive and emotional performance on the subtests, using 2 scores from each task (reflecting average performance level and consistency of performance) and splitting into 3 equal groups of the top, middle, and lowest ⅓ of the group on each of these scores, yielding 26 different scores that were tested with each approach. These splits were assessed based on providing the highest cross-validated accuracy, while applying to the most number of patients and the greatest number of treatment arms.

Step 2: Run prediction modelling, using multivariate logistic regression models, for the full group and each treatment arm, for each splitting approach as described in Step 1. In order to minimize type 1 error rates, only results yielding cross-validated sensitivity or specificity values that were above 0.60 (with the other above 0.55) were considered for evaluation. All regressions were developed on the full sample then cross-validated 50 times using 80% as training set and 20% as testing set.

Predictors were established by taking several individual measures for each task, grouping them according to cognition and emotion task measure categories, then running a 5-factor principle components analysis separately for both categories, resulting in 5 predictor measures within both categories. Separate predictive regression models were then run for main effects alone and for main and interaction effects together, for cognition and emotion task measures run separately and in combination, yielding a total of 6 models for each analysis. This approach was selected due to the large number of predictors, to maximize power to detect small effects. Only the model with the strongest cross-validated results in each case was reported for consideration in the final results.

All results are presented for prediction of symptom remission based on the clinician rated HDRS-17.

Results: For all 3 treatment arms, the strongest predictive results were found when splitting the baseline group based on performance variability for the emotion identification task, into 3 groups based on z-score levels, reflecting low or poor performance (a lot of variability), middle range variability of performance, and high or good performance (very little variability). See table 6 for details.

For escitalopram, predictive models with sufficient accuracy were established for the low (poor) and middle performing groups. The best predictors of remission the low group was in the cognition main effect model with a cross-validated accuracy of 62% (sensitivity 53%, specificity 65%), and for the middle group the best predictors were in the combined emotion main and interaction effects model, yielding a cross-validated accuracy of 60% (sensitivity 56%, specificity 64%). The low range sensitivity level for the low performing group of 53% suggests that this model may be poor at accurately predicting who will remit, but has sufficient specificity to accurately predict those who will not remit.

For sertraline, strong predictions were established for full sample, but with different predictors yielding best remission prediction accuracy for the each of the different low, middle and high performing sub-groups. For the low group, the best predictors of remission were in the combined cognition main and interaction effect model, yielding an accuracy of 67% (sensitivity 58%, specificity 71%). For the middle group, the best predictors were in the emotion main effects model, with an accuracy of 66% (sensitivity 77%, specificity 60%), and for the high performing group the combined emotion main and interactions effects model yielded the best prediction of remission, with an accuracy of 64% (sensitivity 67%, specificity 64%).

For venlafaxine-XR, a remission prediction model with sufficient accuracy was only able to be established for the middle range performing group. This model has an accuracy of 61%, with 50% sensitivity and 62% specificity. The low sensitivity but sufficient specificity performance indicates that this model would be most accurate in predicting those who will not remit with the treatment, rather than those who will remit. The model providing this best prediction consisted on the combined cognition and emotion main effects.

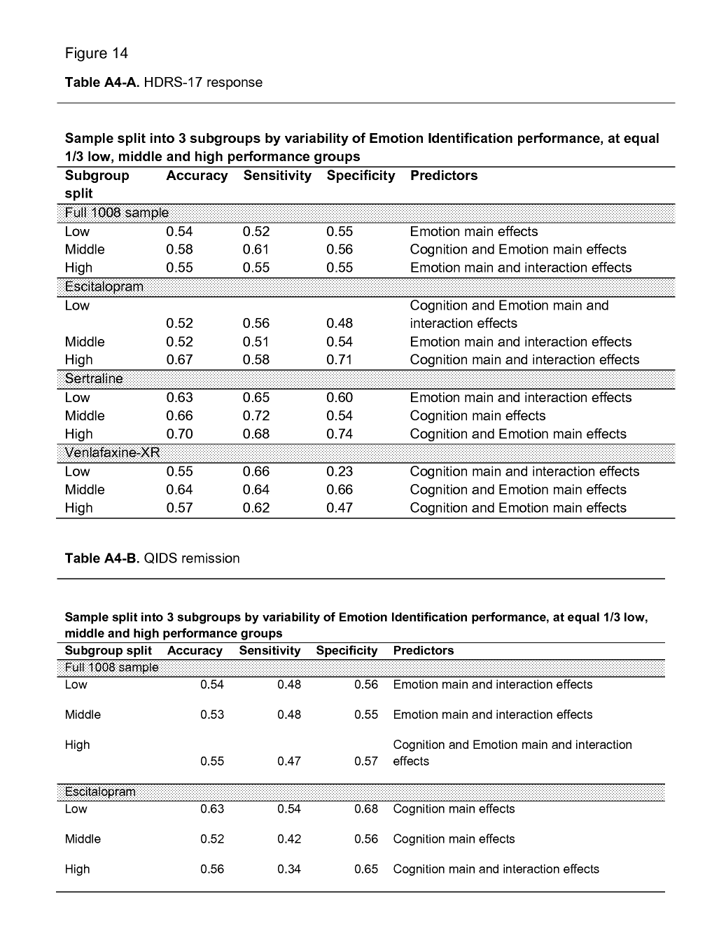

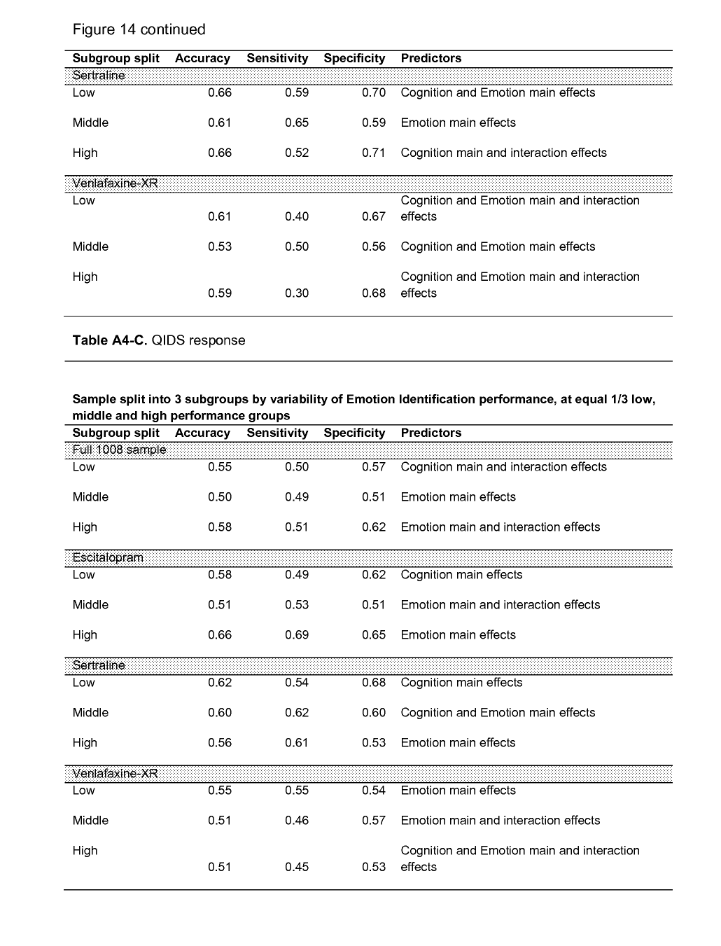

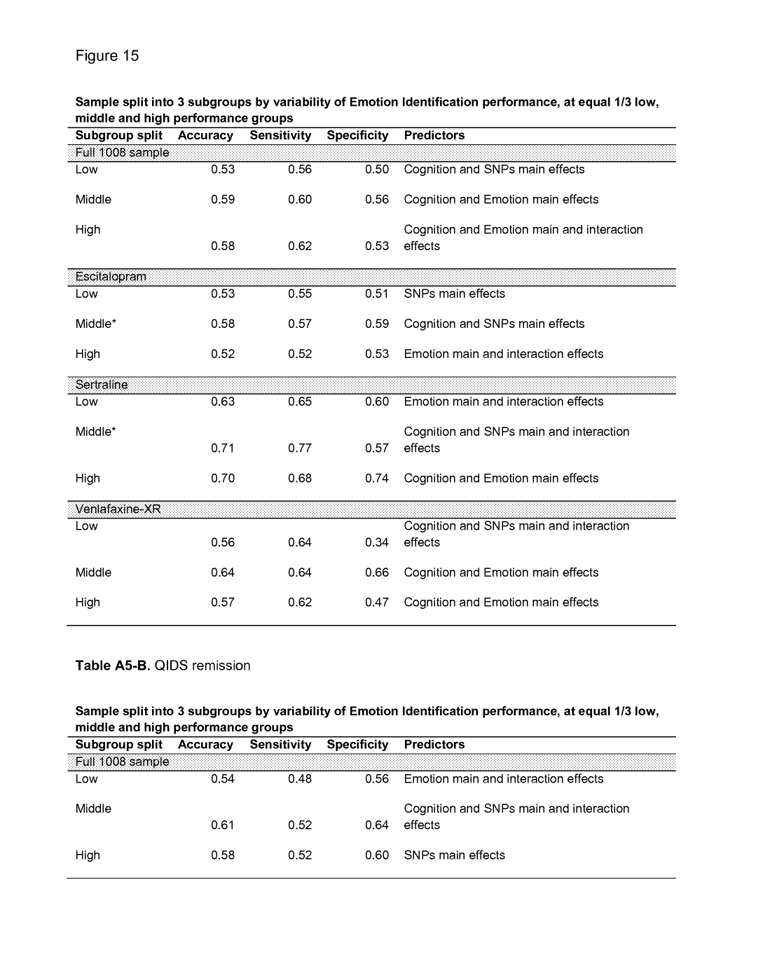

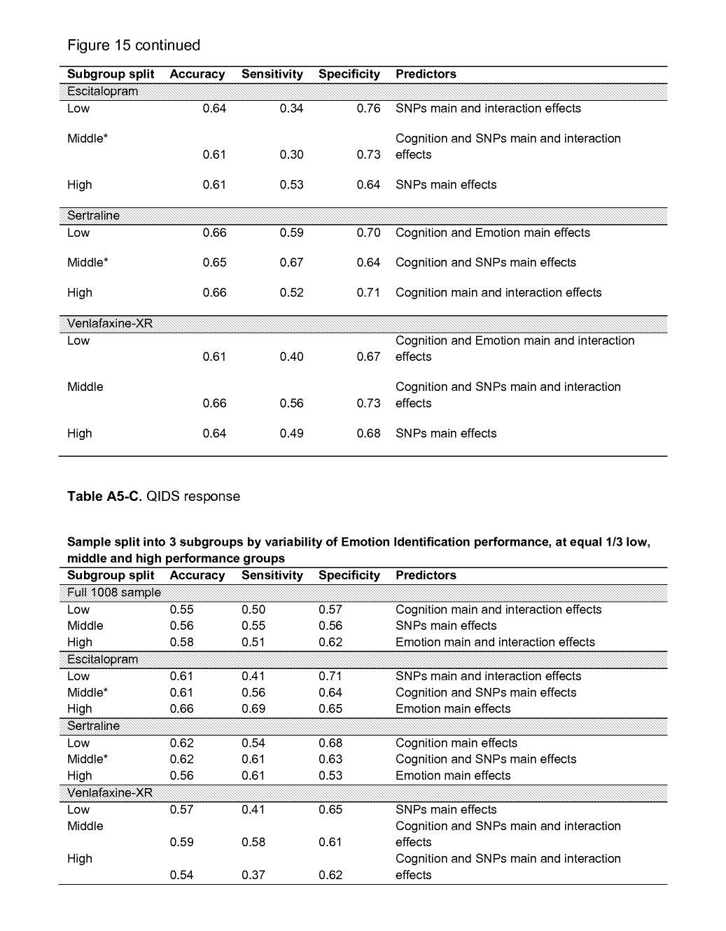

TABLE 8
Treatment outcome results for the prediction model according to
Method 5. Data for outcome measures of HDRS-17 response and
QIDS remission and response are provided in FIG. 14.
Sample split into 3 subgroups by variability of Emotion Identification
performance, at equal ⅓ low, middle and high performance groups

| | | | | |
|---|---|---|---|---|
| Sub- | | | | |
| group | | | | |
| split | Accuracy | Sensitivity | Specificity | Predictors |

Full 1008 sample

| | | | | |
|---|---|---|---|---|
| Low | 0.54 | 0.52 | 0.55 | Emotion main effects |
| Middle | 0.58 | 0.61 | 0.56 | Cognition and Emotion |
| | | | | main effects |
| High | 0.55 | 0.55 | 0.55 | Emotion main and |
| | | | | interaction effects |

Escitalopram

| | | | | |
|---|---|---|---|---|
| Low | .62 | .53 | .65 | Cognition main effects |
| Middle | .60 | .56 | .64 | Emotion main and |
| | | | | interaction effects |
| High | 0.57 | 0.50 | 0.60 | Emotion main and |
| | | | | interaction effects |

Sertraline

| | | | | |
|---|---|---|---|---|
| Low | .67 | .58 | .71 | Cognition main and |
| | | | | interaction effects |
| Middle | .66 | .77 | .60 | Emotion main effects |
| High | .64 | .66 | .64 | Emotion main and |
| | | | | interaction effects |

Venlafaxine-XR

| | | | | |
|---|---|---|---|---|
| Low | 0.61 | 0.44 | 0.67 | Cognition main and |
| | | | | interaction effects |
| Middle | 0.54 | 0.46 | 0.59 | Emotion main and |
| | | | | interaction effects |
| High | 0.54 | 0.46 | 0.59 | Emotion main and |
| | | | | interaction effects |

### Tests Based on Genomic Parameters to Predict Treatment Outcomes in MDD

In one or more further embodiments the present invention relates to a test method based on an MDD patient's genomic make-up. The test is useful to predict the treatment outcome and/or response of treating MDD with a particular ADM for each individual patient and therefore is useful in assisting the practitioner to prescribe the most effective ADM at the onset of treatment.

Furthermore, the invention relates to test methods useful in predicting the treatment outcome achieved with a particular ADM in MDD. The test is based on the combination of an assessment of an MDD patient's performance in relation to general cognition and to emotional cognition tasks with the patient's genomic make-up. The test is therefore useful in assisting the practitioner to identify and prescribe the most effective ADM for each individual patient at the onset of treatment.

Assessment of a Patient's Genomic Make-Up

For the assessment of their genomic make-up and to identify particular genomic parameters of treatment outcome to a particular ADM, potential participants in the clinical study provided blood samples. The blood samples were then treated with Ethylenediaminetetraacetic acid (EDTA) and genomic DNA was extracted using the Puregene DNA extraction method (Qiagen, Valencia, Calif.).

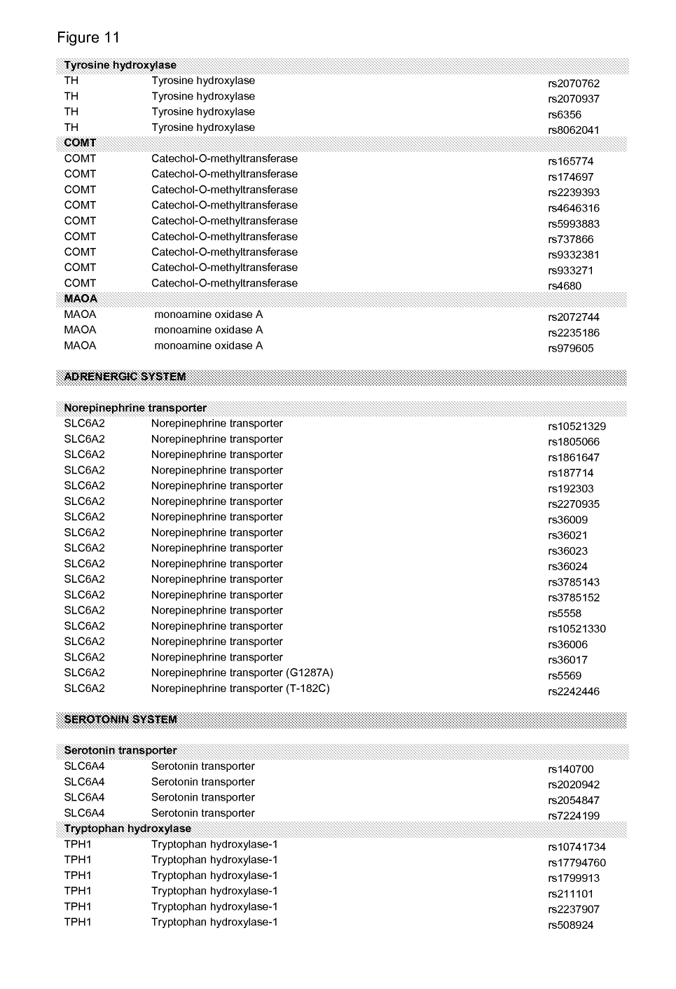

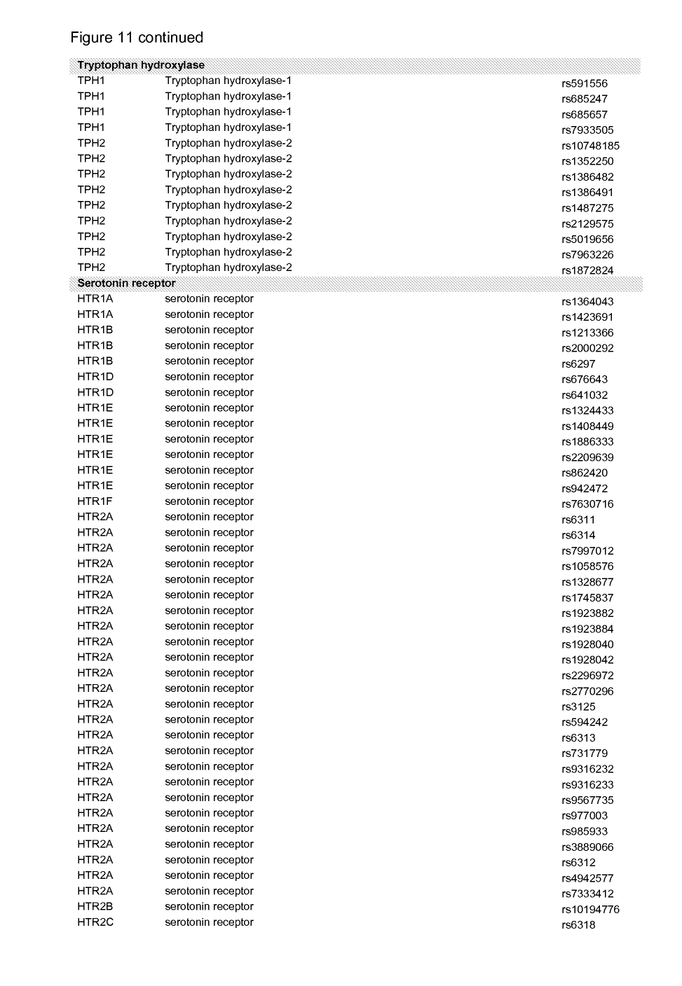

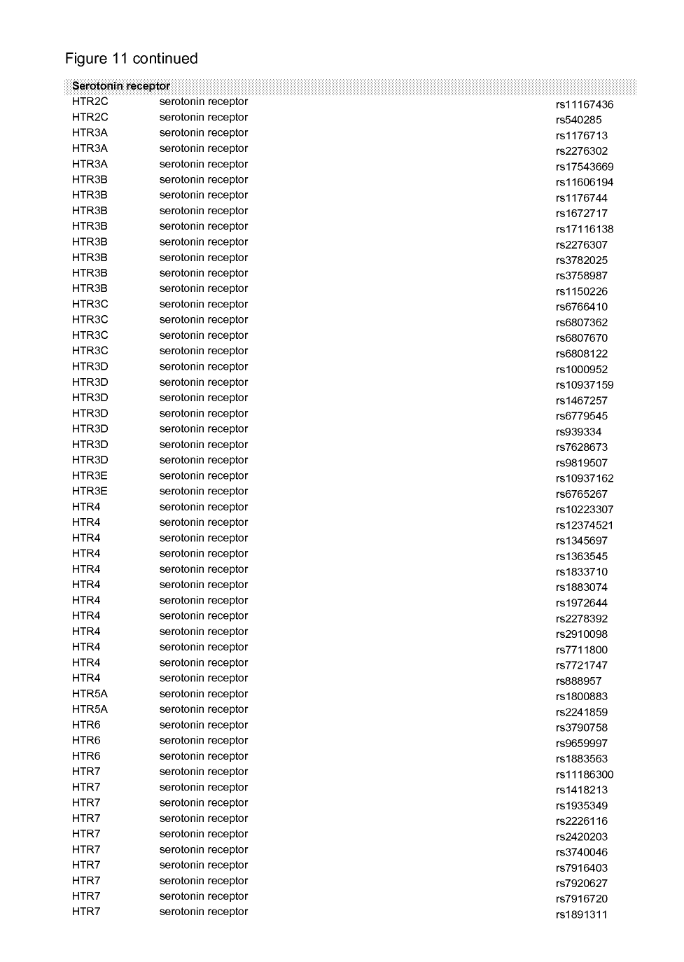

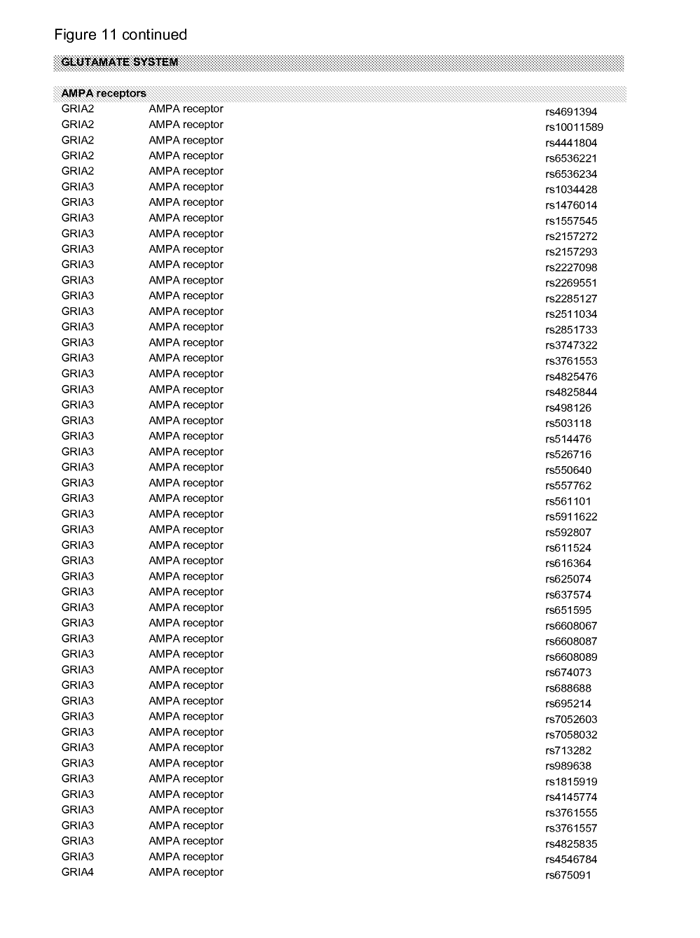

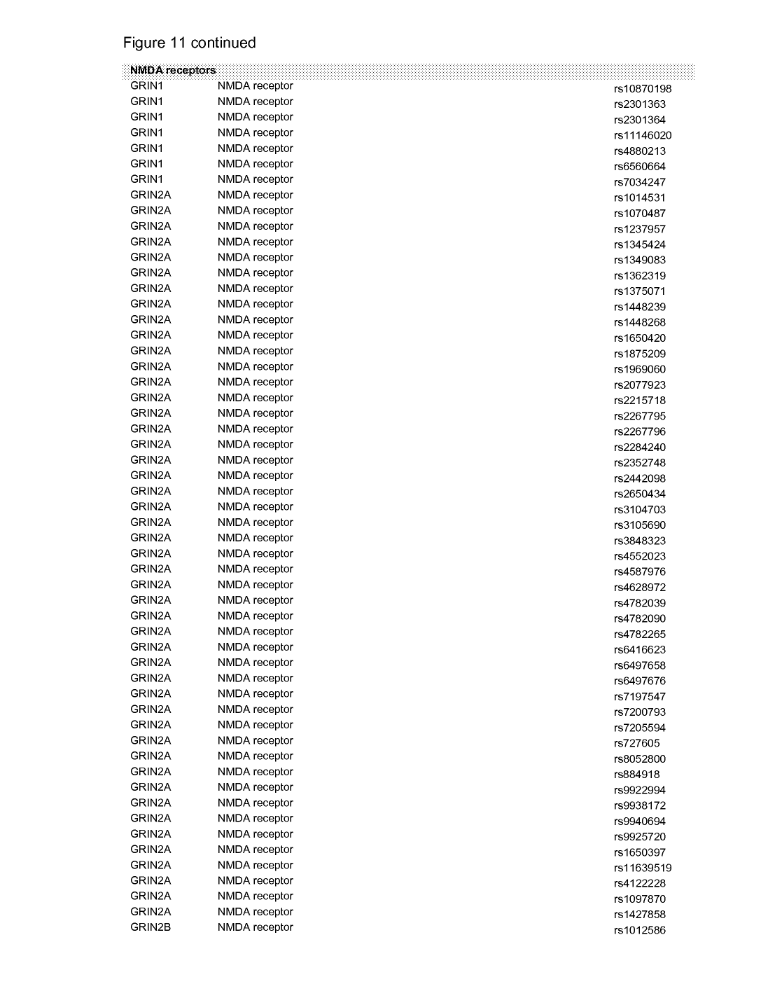

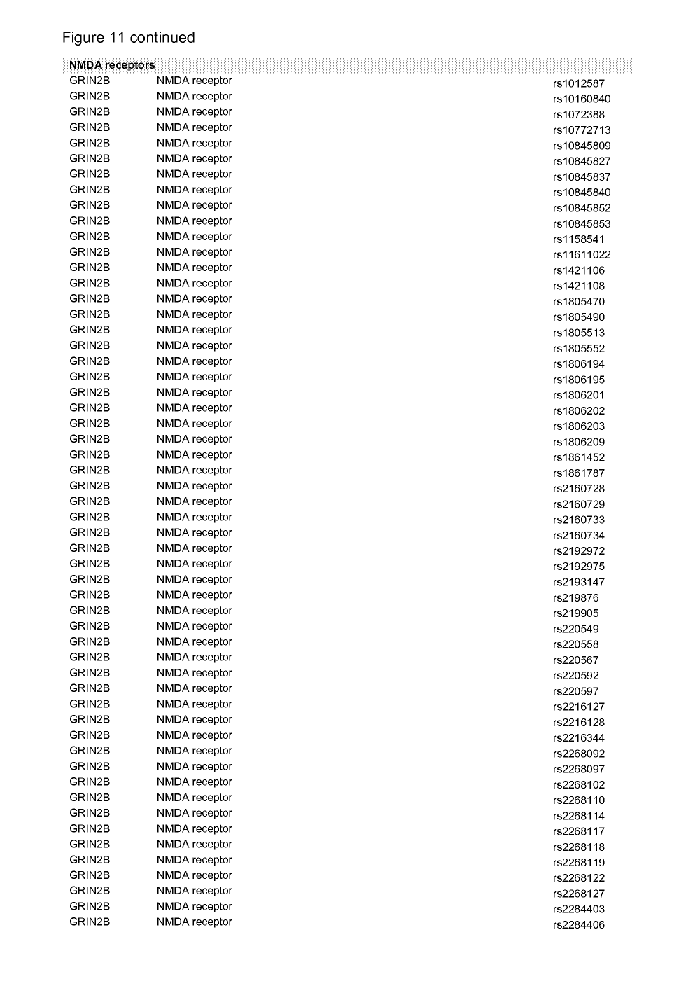

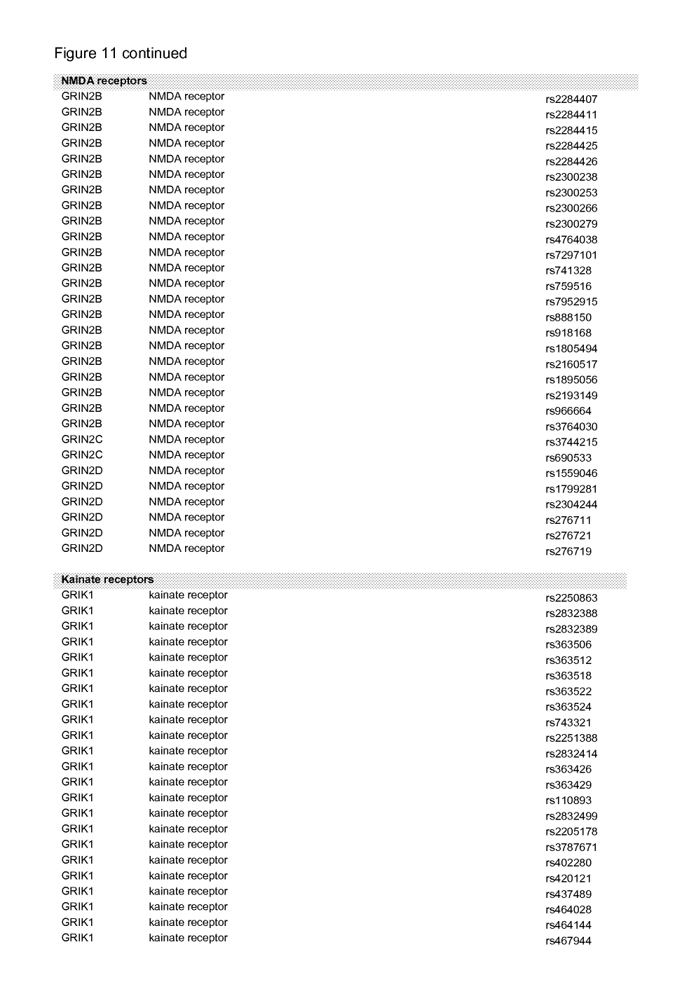

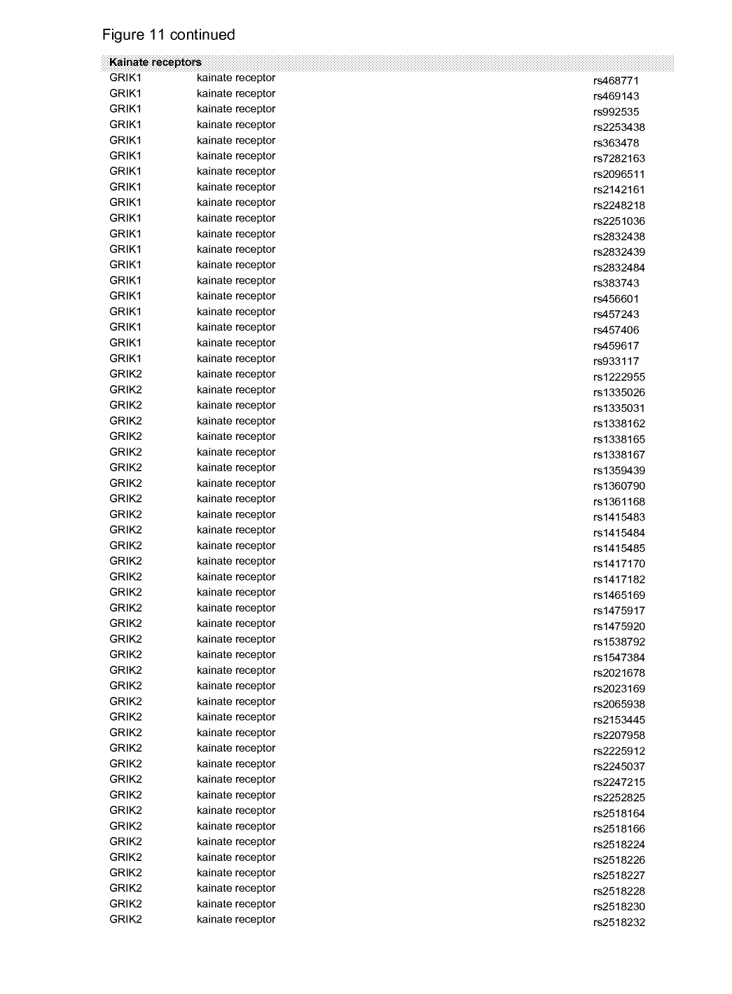

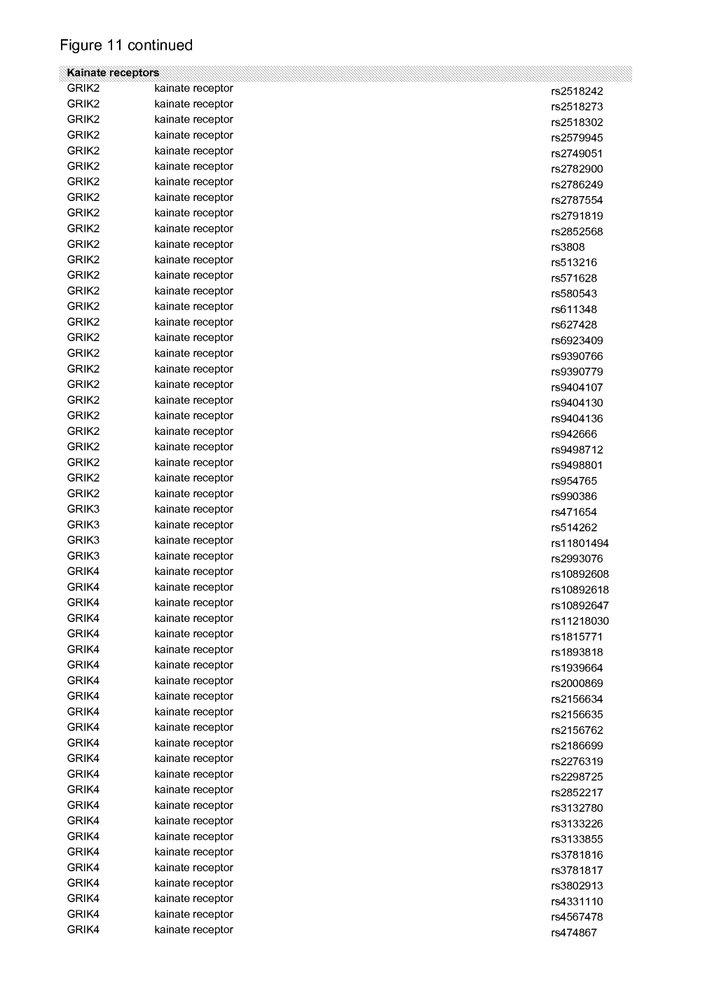

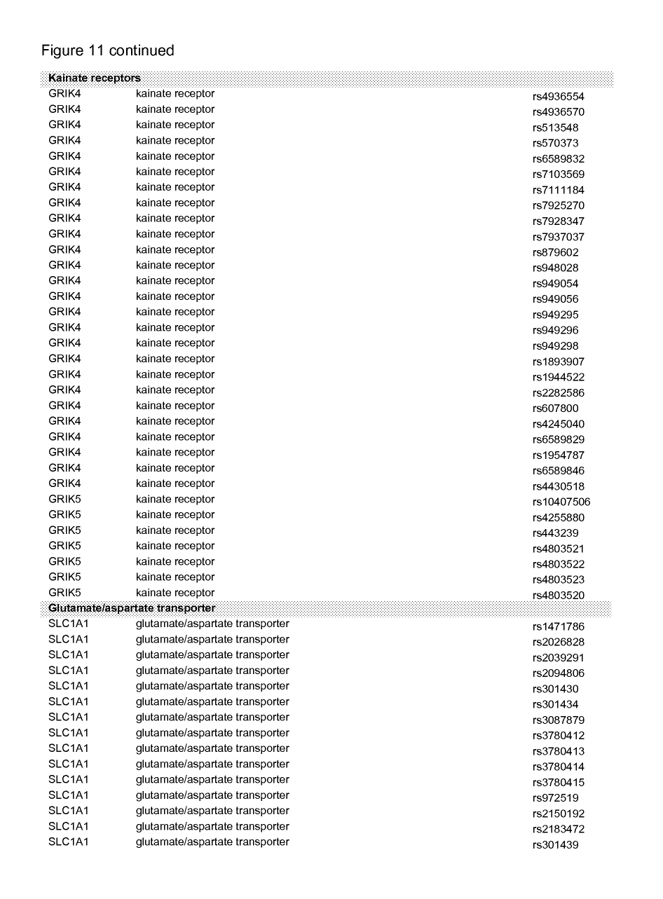

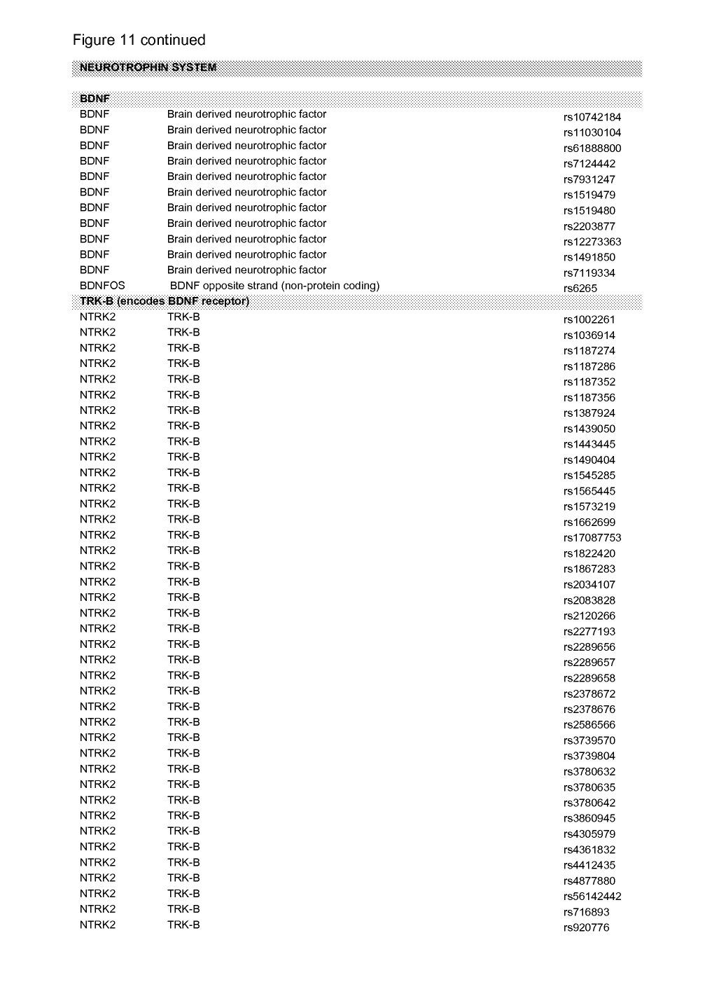

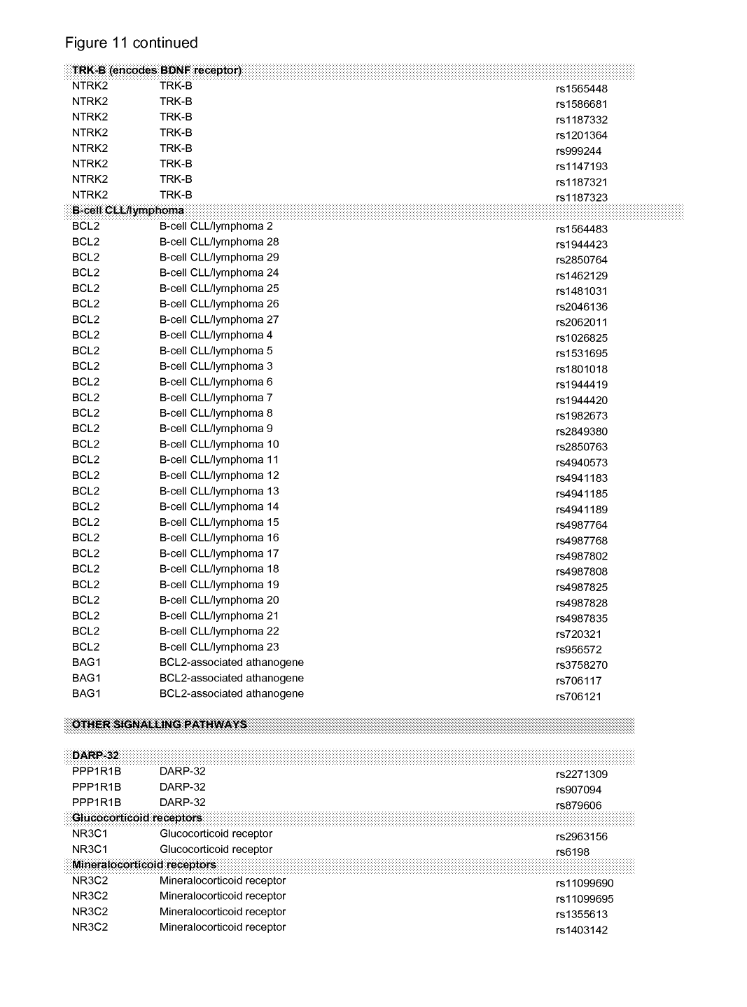

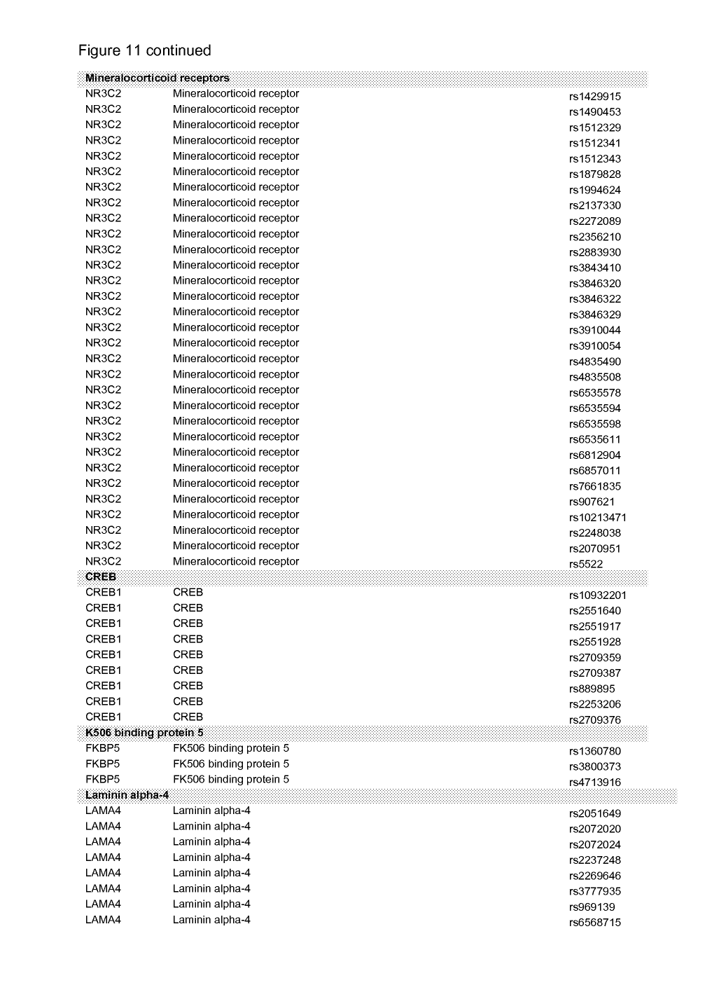

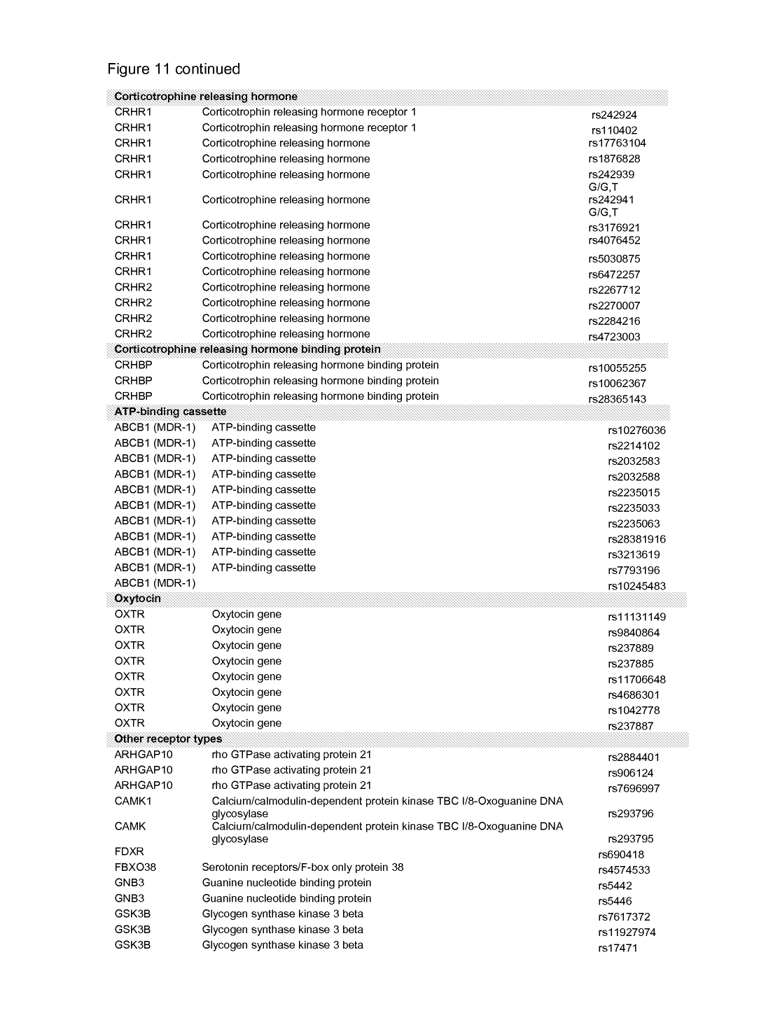

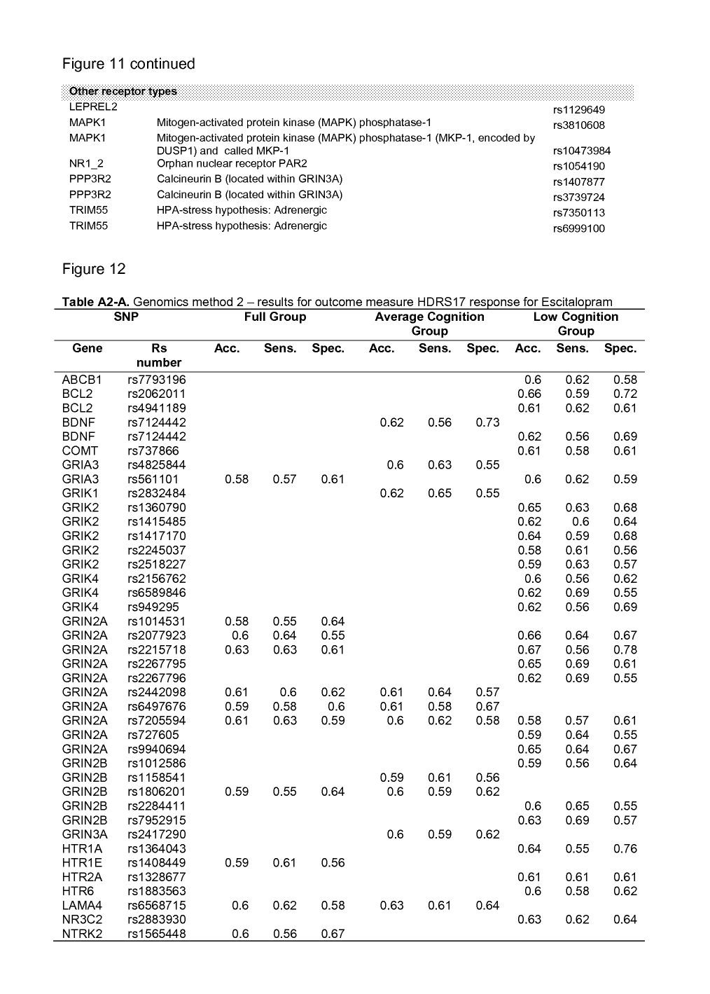

The extracted DNA was subjected to genotyping using the Illumina VeraCode Golden Gate SNP genotyping platform (Illumina, Hayward, Calif.) by Covance, Inc. Two custom oligo panels containing 740 SNPs were utilized. The complete set of genomic parameters assessed (see FIG. 11) comprised SNPs of genomic loci suggested as being implicated in MDD by the STAR*D study (McMahon et al., 2006) as well as additional SNPs of genomic loci otherwise implicated in depression to ADMs.

Initial quality control (QC) analysis of the genotyping results was performed by Covance, Inc., according to standard criteria (Supplemental Data; attach Covance QC document). Of 740 SNPs, 33 were excluded by Covance at the genotyping stage due to signal intensities or poor separation of clusters, which left 707 SNPs for further analysis. Of 1210 patient samples initially submitted for DNA extraction, 31 were excluded from further consideration due to poor call rates across the entire SNP set. For the present analysis, DNA samples from the 1008 participants in the clinical study who were assigned to each of the 3 treatment arms (336 per arm) were analysed.

To further screen for potential genotyping errors in the SNPs that passed initial QC analysis, the deviation from the Hardy Weinberg equilibrium (HWE) was determined across the dataset (Hosking et al., 2004). Because the sample was ethnically heterogeneous, and since population stratification can result in deviation from HWE, we tested for HWE in the 773 subjects with self-reported white ethnicity. For SNPS linked to the X chromosome (X-linked), we used only the white female subjects. Haploview (v 4.2) (Barrett et al., 2005) was used to calculate p-values for the deviation from the HWE. Fourteen SNPs were identified that deviated from HWE such that the associated p-value was less than 0.00007 (=0.05/737; the Bonferroni corrected p-value for the 707 SNPs in the analysis). Genotyping data plots were examined for these SNPs, and all were excluded based on skewed or poorly separated clusters leaving 693 SNPs for further analysis.

### Method 1

Design:

Univariate linear regression analyses were undertaken on individual SNP polymorphisms, for HDRS-17 percent change and remission-non remission, separately for each treatment arm. In each of the models, only one SNP was used as a predictor. However, there were several covariates in the model (coefficients and p-values not reported). These were race (Caucasian vs. non-Caucasian), gender, age (rounded to the nearest decade), BMI, final daily dose of treatment medication, treatment arm, number of weeks completing the study, and clinical site.

Only regression models significant at a level of p<0.01 were considered a statistically significant outcome, in order to correct for multiple comparisons and limit the possibility of false positive results.

Results:

For escitalopram alone, 2 SNPs of the serotonergic tryptophan hydroxylase TPH2 gene were found to be significantly predictive of treatment outcome. For SNPs rs1386491 and rs2129575, the minor alleles G and T, respectively, were predictive of both greater percentage improvement on symptom scores and of achieving symptom remission.

For venlafaxine-XR alone, 2 SNPs of the Brain Derived Neurotrophic Factor (BDNF) gene (rs2203877 and rs1519479) were significantly predictive of achieving symptom remission, with alleles C and T, respectively, predicting greater chance of symptom remission.

Finally, the serotonin gene HRT2A SNP rs985933 was predictive of percent improvement on symptom scores for both escitalopram and venlafaxine-XR treatment arms, with the T allele predicting both smaller improvement on escitalopram and greater improvement on venlafaxine-XR.

Conclusion: Particular genomic parameters, specifically particular SNPs for the TPH2, BDNF and HRT2A genes are useful predictors of treatment outcome in MDD for escitalopram and venlafaxine-XR.

### Method 2

Individual genomic SNP assessment scores can be used to predict symptom remission for individual treatment arms, with up to 69% sensitivity and 60% specificity for escitalopram, 66% sensitivity and 61% specificity for Escitalopram, and 70% sensitivity and 67% specificity for venlafaxine-XR.

Design:

The aim of this method was to identify which individual SNPs are associated with treatment outcome in a simple and direct analysis approach, in the full sample and after splitting the group based on cognitive performance, as a method of taking the real world variation in the depression construct (i.e. in patients with depression) into account.

Univariate logistic regression modelling was used for prediction modelling, with separate univariate models run for each of the 687 SNPs, incorporating cross-validation using a k-fold approach known in the art. Each regression was run 30 times, each time using a new random 80% of the sample as a “training” set, and 20% as a “test” set. All of the results presented are these cross-validation values. In order to further minimize type 1 error rates, only results yielding cross-validated sensitivity or specificity values that were above 0.60 (with the other above 0.55) were considered for evaluation.

First, the sample was stratified as described at [0116] above in Step 1 of Method 2 of the tests based on cognitive parameters to predict treatment outcomes in MDD (see FIG. 5). Again, all subsequent steps were run separately for these cognition level sub-groups, within each of the three treatment arms, resulting in a total of six sub-groups for which prediction modelling was performed.

According to the methods of the present invention, the threshold for considering the prediction of treatment outcome to be clinically meaningful (when considering how treatment regimes are currently decided upon in clinical practice) is that classification is ≧10% better than chance for symptom response/non-response and symptom remission/non-remission classifications (i.e. either sensitivity or specificity>=0.6).

Results:

For all treatment arms, stronger predictions were found by splitting the group based on baseline cognitive performance than for the full group (see Tables 9 to 11 for detailed results).

For escitalopram, prior to splitting on cognitive performance 5 SNPs were found to predict clinician-rated symptom remission, with up to 69% sensitivity for the serotonin receptor HTR1D gene rs641032 SNP (with 55% specificity), and up to 60% specificity for the serotonin receptor HTR2A gene rs2770296 SNP (with 61% sensitivity; see Table 9).

When split by baseline cognition, for escitalopram 13 SNPs were found to predict symptom remission for the average cognition group, and 20 SNPs for the below average cognition group, with the strongest predictions being from glutamate system receptor genes. For the average cognition group, symptom remission was predicted with up to 66% accuracy (63% sensitivity, 67% specificity) for glutamate kainate receptor GRIK2 gene rs2518227 SNP. For the below average cognition group, symptom remission was predicted with up to 75% sensitivity for the glutamate kainate receptor GRIK2 gene rs1415485 SNP (65% accuracy; 75% sensitivity; 62% specificity), and up to 76% specificity for the glutamate NMDA receptor GRIN1 gene rs4880213 SNP (71% accuracy; 63% sensitivity; 76% specificity; see Table 9).

For sertraline, prior to splitting on cognitive performance 4 SNPs were found to predict clinician-rated symptom remission, with up to 66% sensitivity for the Mineralocorticoid receptor NR3C2 gene rs1355613 SNP (with 59% specificity), and up to 61% specificity for the Neurotrophin system B-cell CLL/lymphoma BCL2 gene rs4987802 SNP (with 55% sensitivity; see Table 10).

When split by baseline cognition, for sertraline 12 SNPs were found to predict symptom remission for the average cognition group, and 33 SNPs for the below average cognition group. For the average cognition group, symptom remission was predicted with up to 67% accuracy (66% sensitivity, 68% specificity) for the ATP-binding cassette ABCB1 (MDR-1) gene rs7793196 SNP. For the below average cognition group, the strongest predictions for symptom remission were with Neurotrophin system B-cell CLL/lymphoma BCL2 gene SNPs, with 88% sensitivity for the rs1564483 SNP (accuracy 64%; sensitivity 88%; specificity 55%), and 84% specificity for the rs1944420 SNP (78% accuracy; 61% sensitivity; 84% specificity; see Table 10).

TABLE 9
Escitalopram results. Results shown where sensitivity or specificity is
greater than 60% and the other greater than 55%. Strong results are considered to be
those where both sensitivity and specificity are greater than 60%, and are highlighted and
shown in italics. Only results meeting these criteria are displayed in the table, non-
significant results are not shown.

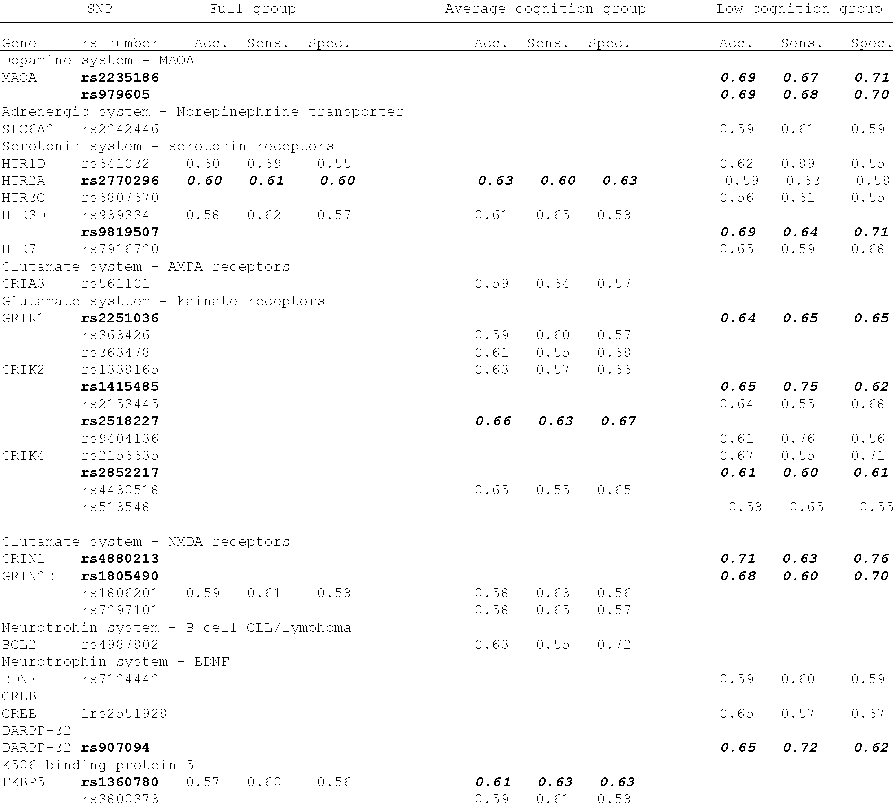

For venlafaxine-XR, prior to splitting on cognitive performance 16 SNPs were found to predict clinician-rated symptom remission, 8 of which are glutamate system kainite receptors, which predicted remission only for venlafaxine and not for the other treatment arms. Symptom remission for venlafaxine-XR was predicted with maximal sensitivity and specificity by serotonin receptor HTR5A gene SNPs, with up to 70% sensitivity for SNP rs2241859 (with 55% specificity), and up to 67% specificity for SNP rs1800883 (with 55% sensitivity; see Table 11).

TABLE 10
Sertraline results. Results shown where sensitivity or specificity is
greater than 60% and the other greater than 55%. Strong results are considered to be
those where both sensitivity and specificity are greater than 60%, and are highlighted and
shown in italics. Only results meeting these criteria are displayed in the table, non-
significant results are not shown.

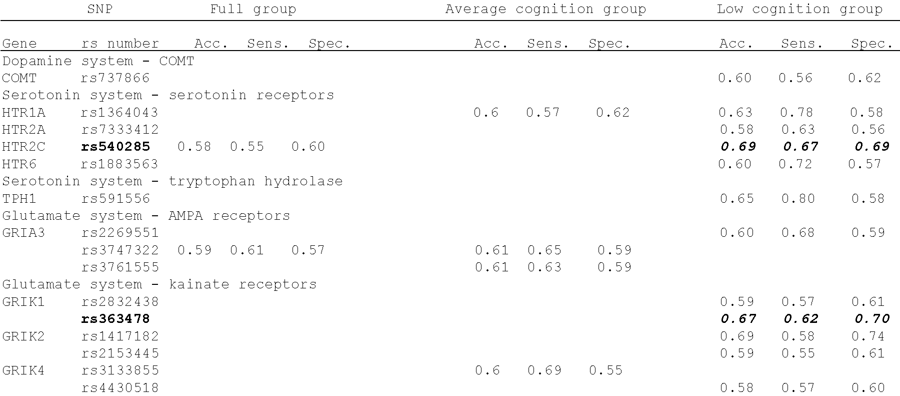

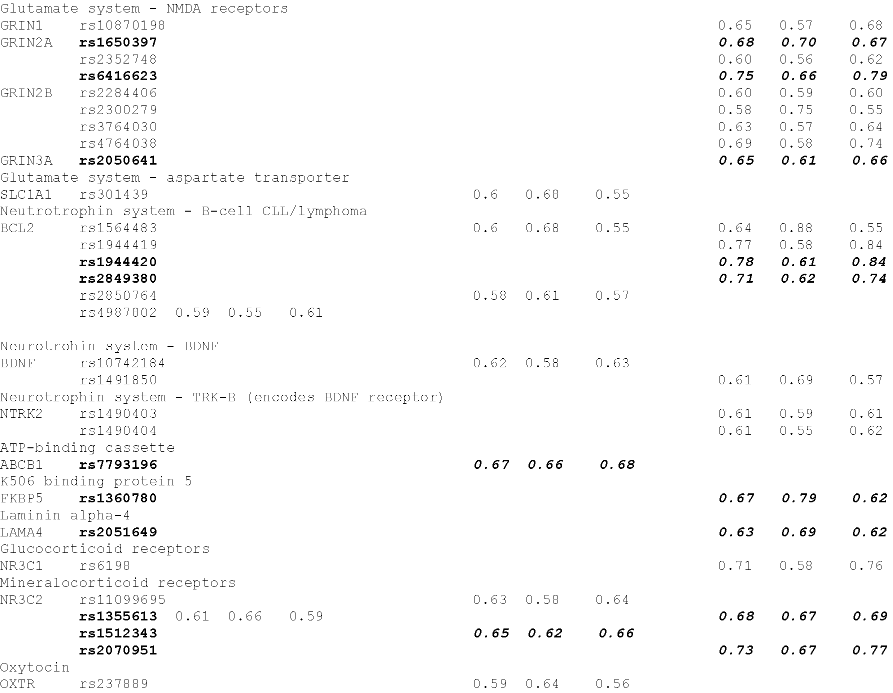

When split by baseline cognition, for venlafaxine-XR 13 SNPs were found to predict symptom remission for the average cognition group, and 44 SNPs were found to predict symptom remission for the below average cognition group. For the average cognition group, symptom remission was predicted with up to 62% accuracy (60% sensitivity; 62% specificity) for the glutamate system AMPA receptor GRIA3 gene rs3747322 SNP. For the below average cognition group, symptom remission was predicted with up to 80% sensitivity for the glutamate system AMPA receptor GRIA3 gene rs688688 SNP (72% accuracy; 80% sensitivity; 68% specificity), and up to 77% specificity for the glutamate system NMDA receptor GRIN2A gene rs1448239 SNP (69% accuracy; 56% sensitivity; 77% specificity; see Table 11).

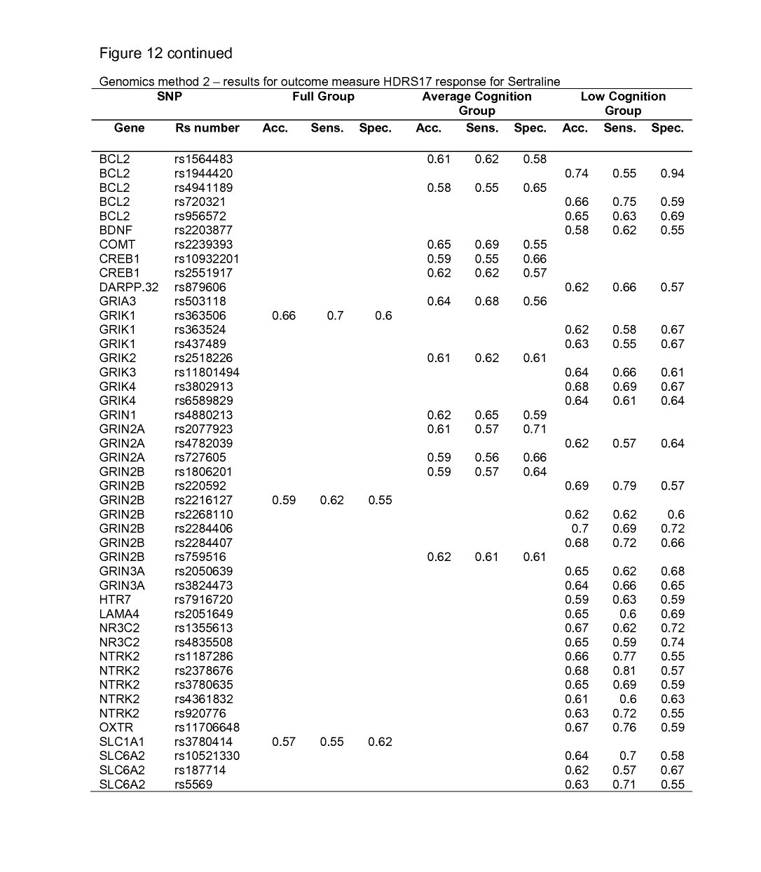

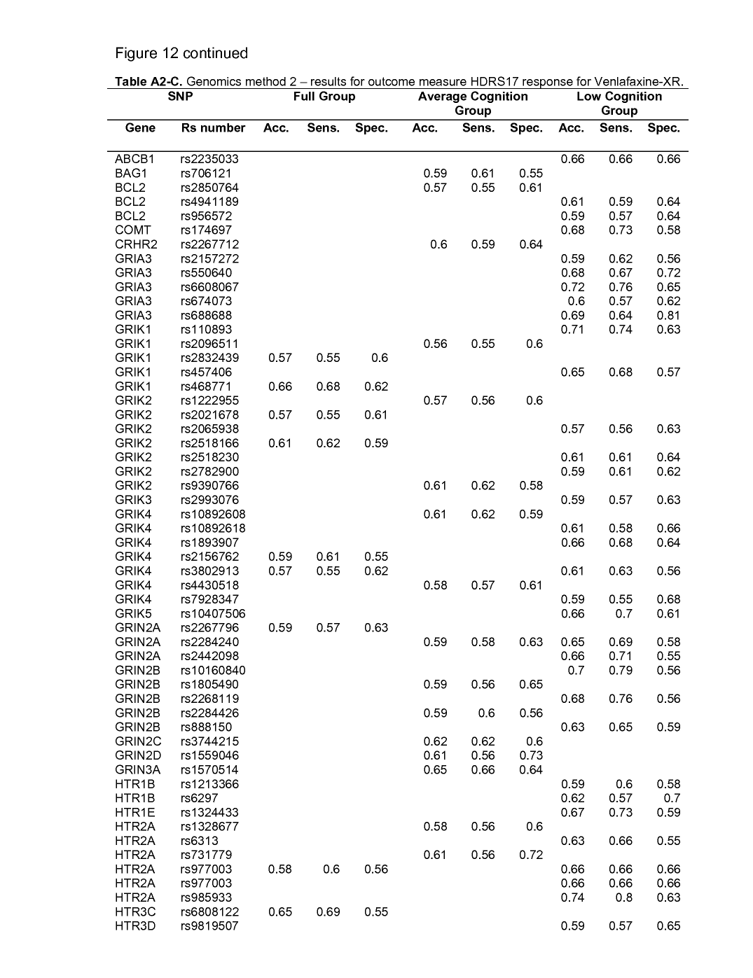

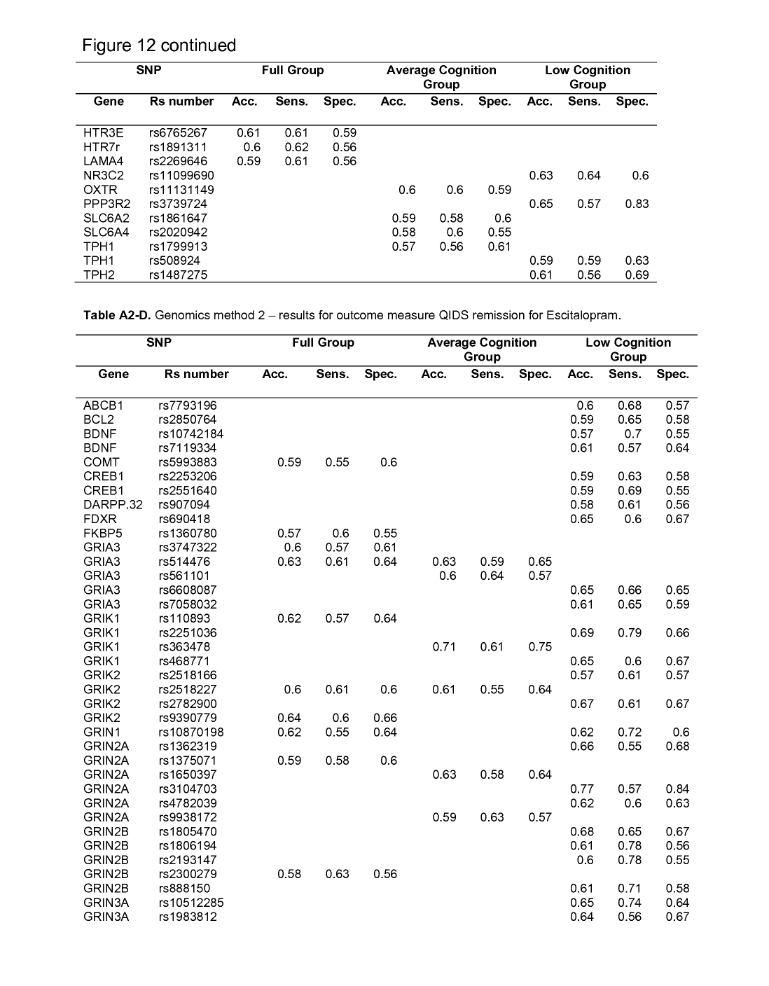

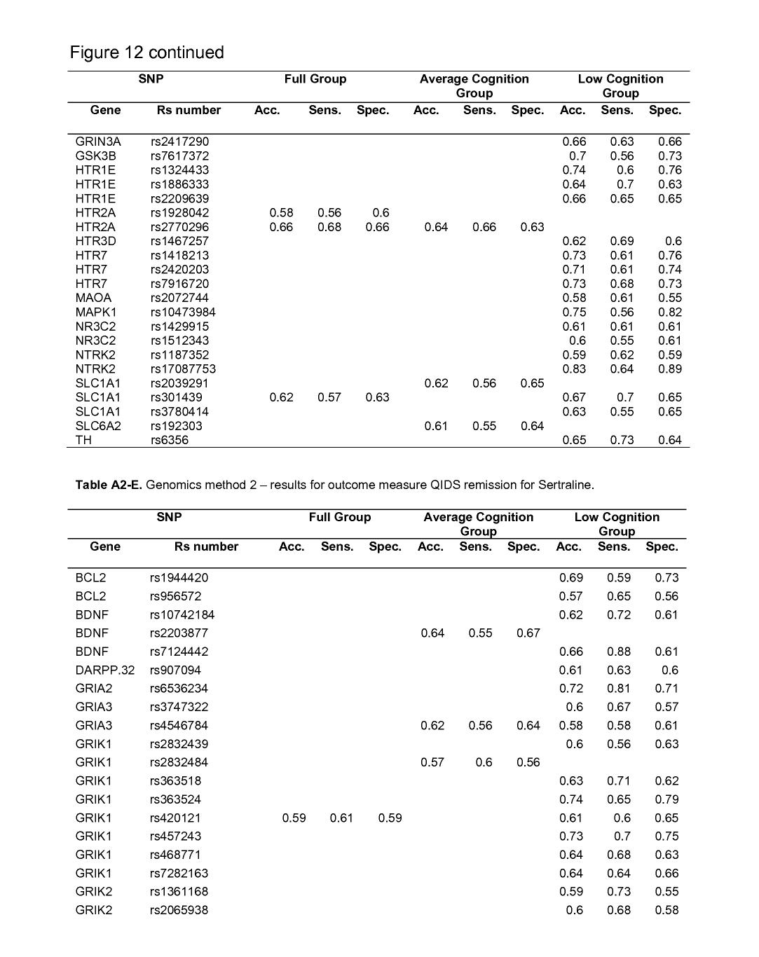

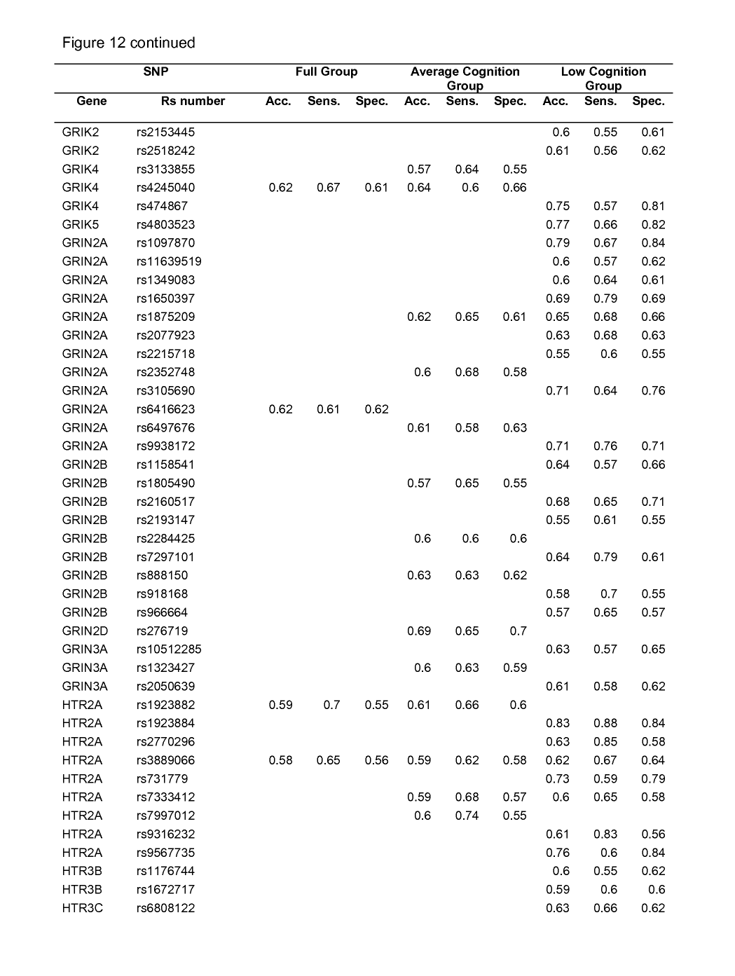

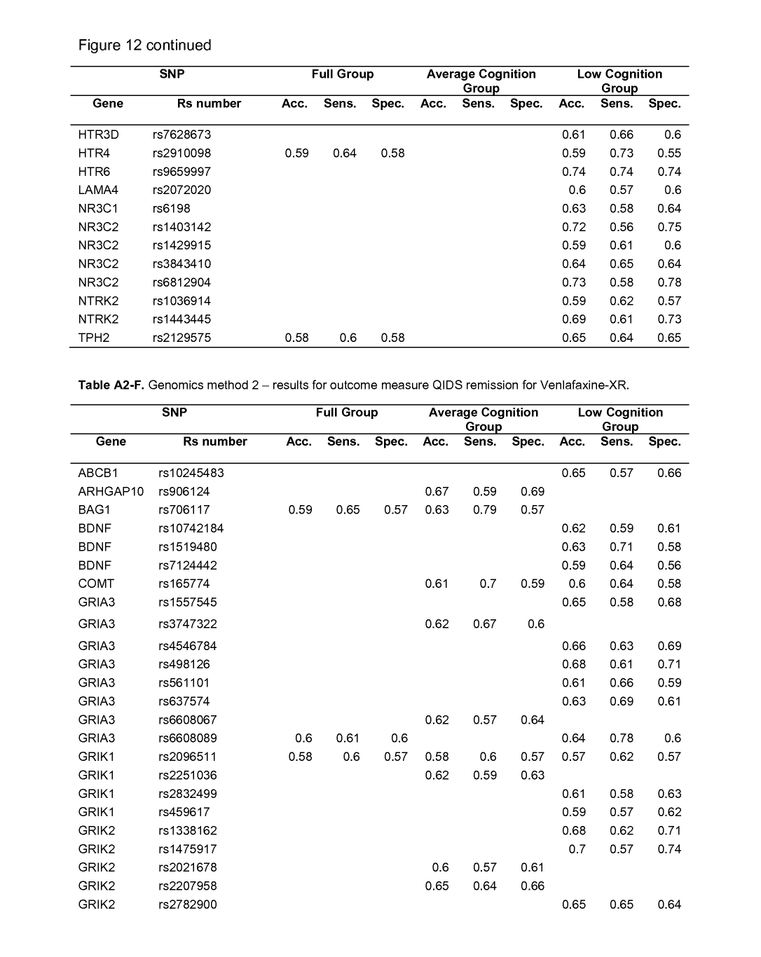

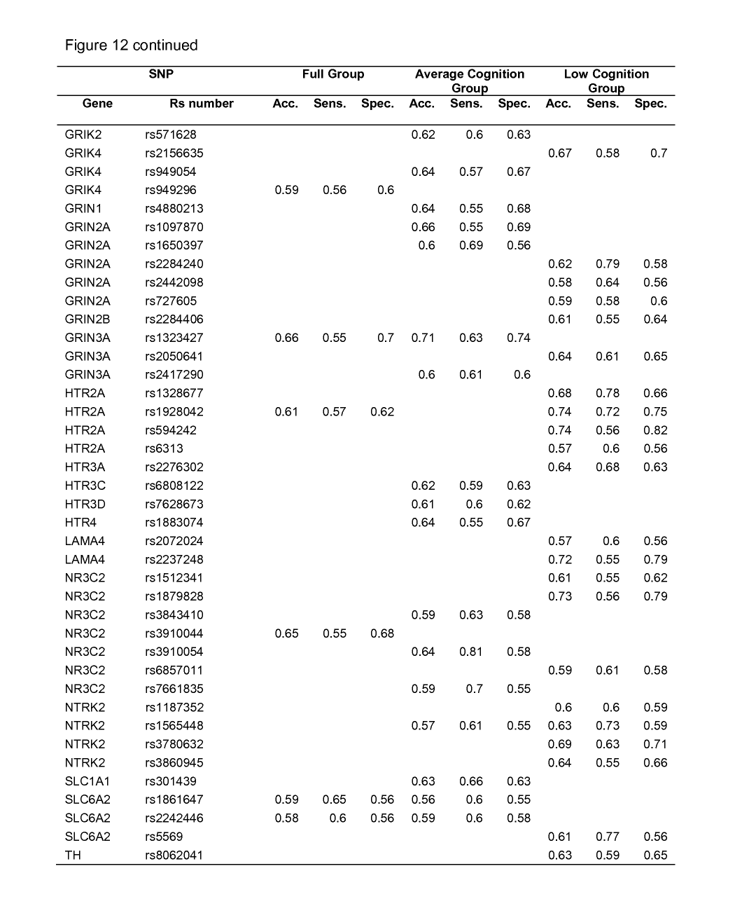

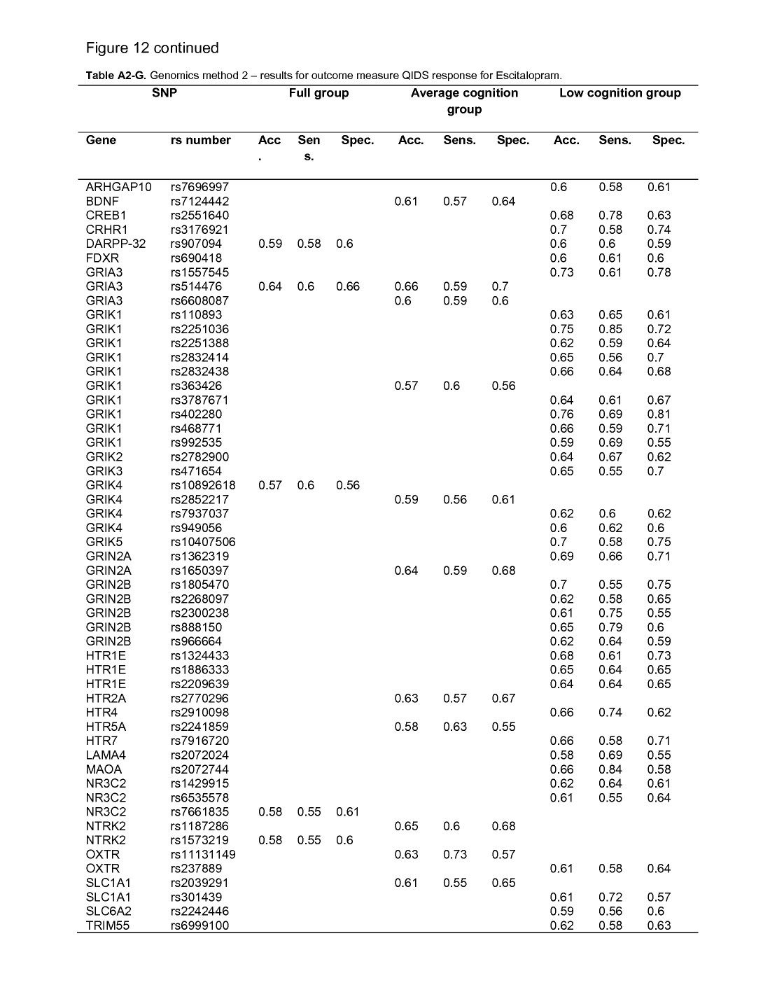

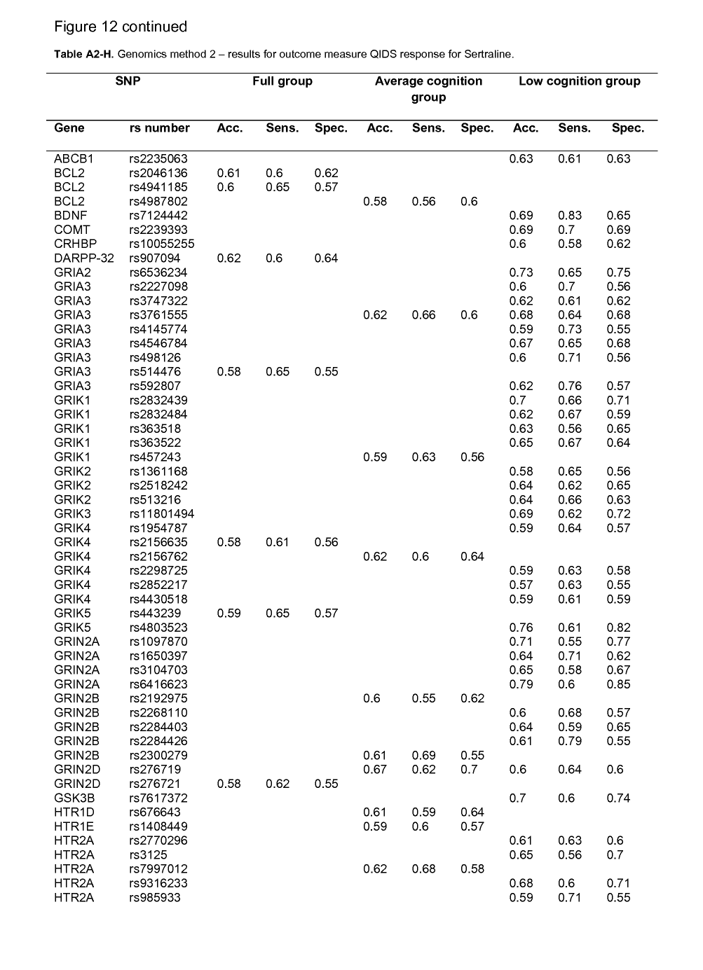

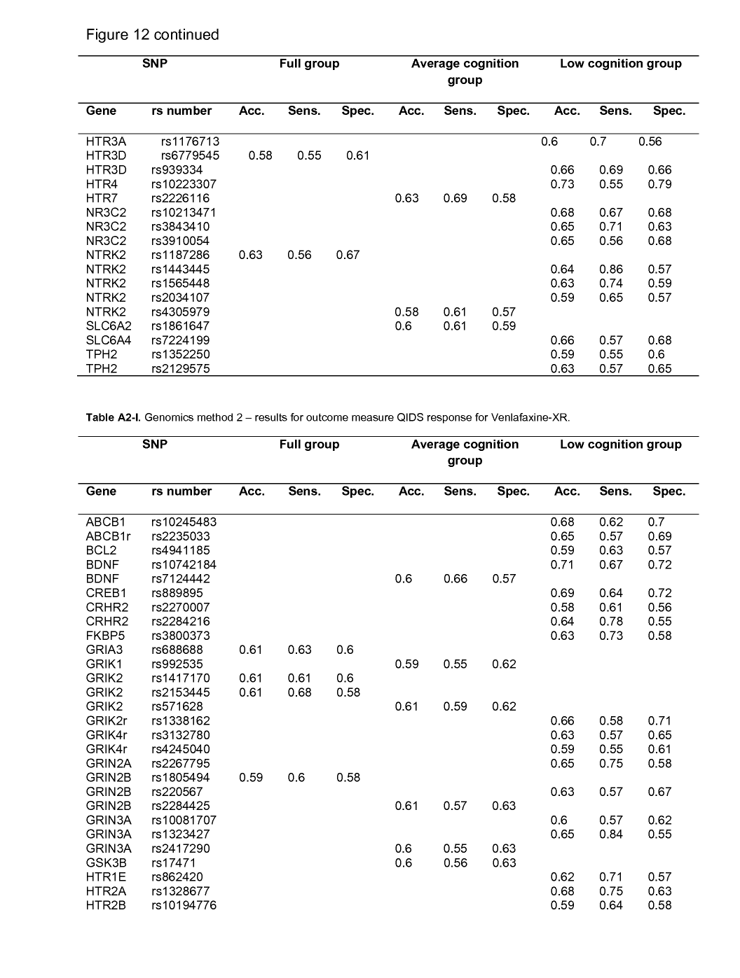

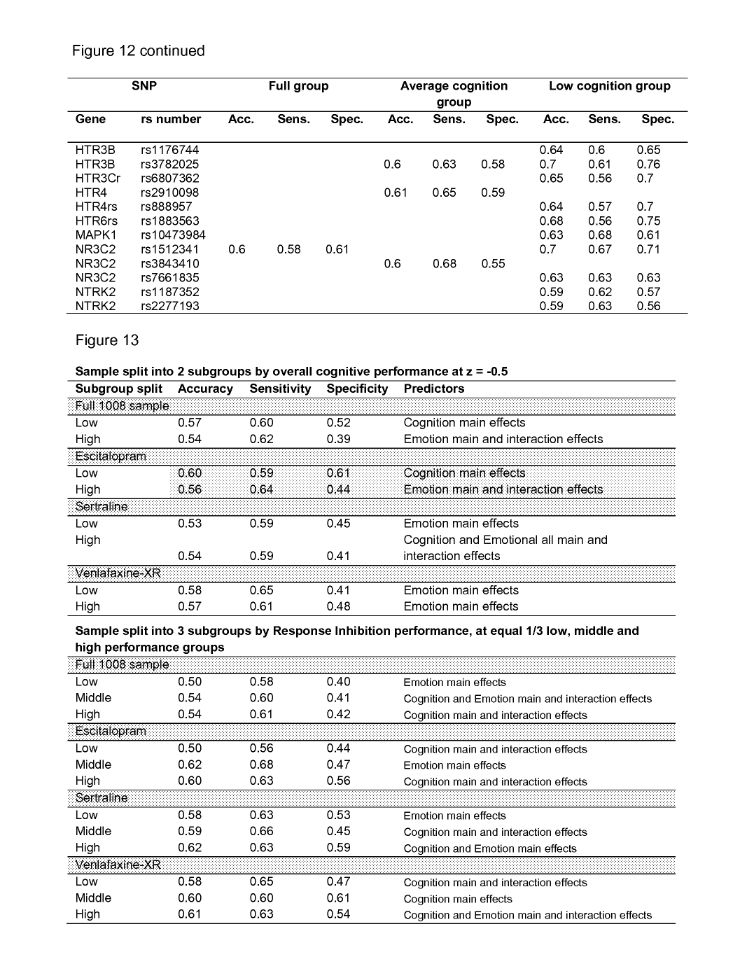

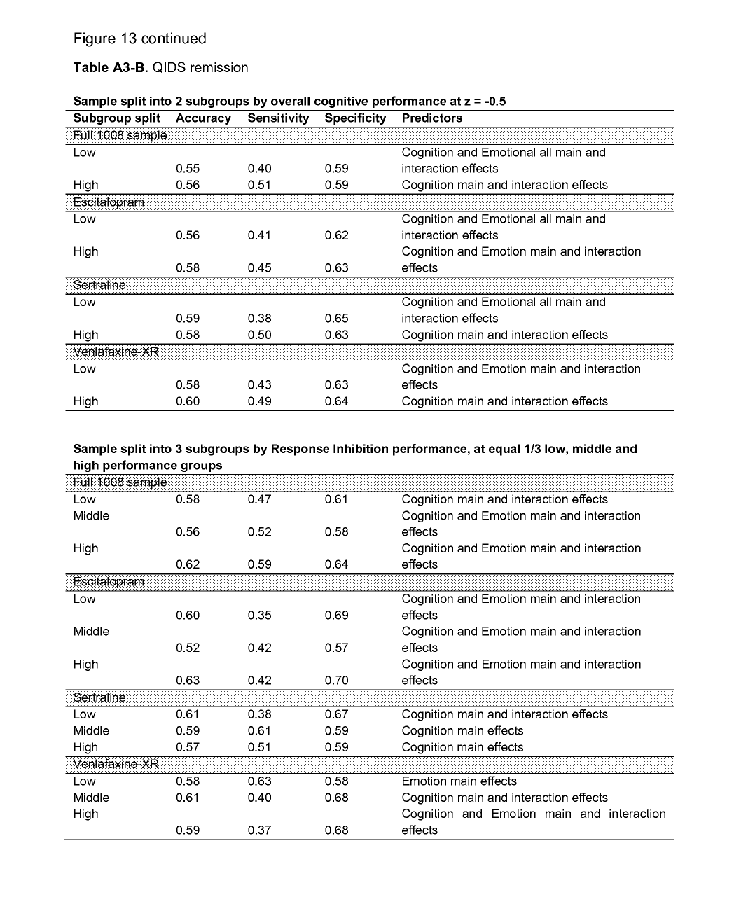

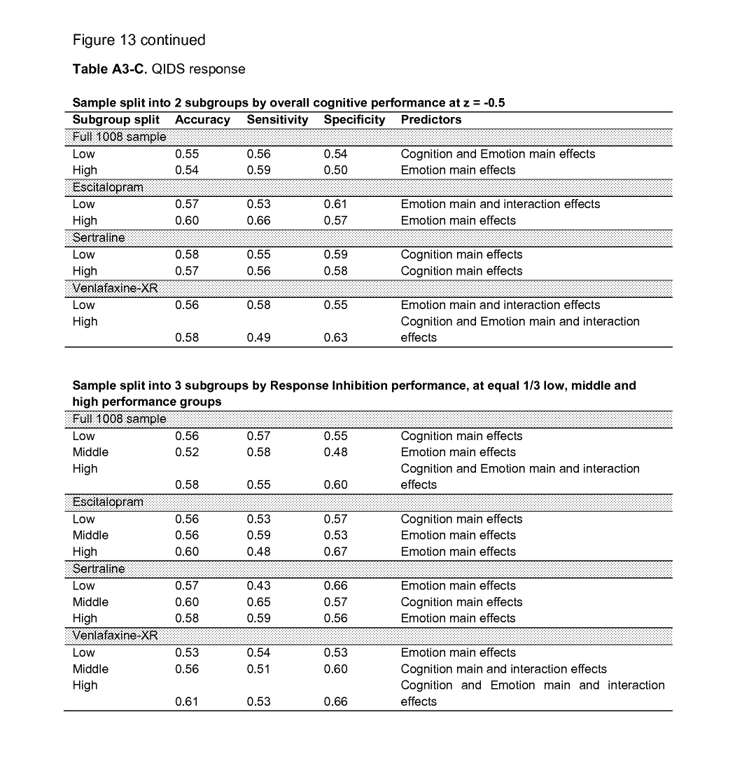

Predictive models were also assessed for symptom outcome measured by symptom response, defined as a ≧50% decrease from the baseline score for HRSD₁₇ and QIDS-SR₁₆, and symptom remission, defined as a score of ≦5 on the QIDS-SR₁₆. Detailed results are provided in FIG. 12.

TABLE 11
Venlafaxine-XR results. Results shown where sensitivity or specificity
is greater than 60% and the other greater than 55%. Strong results are considered to be
those where both sensitivity and specificity are greater than 60%, and are highlighted and
shown in italics. Only results meeting these criteria are displayed in the table, non-
significant results are not shown.

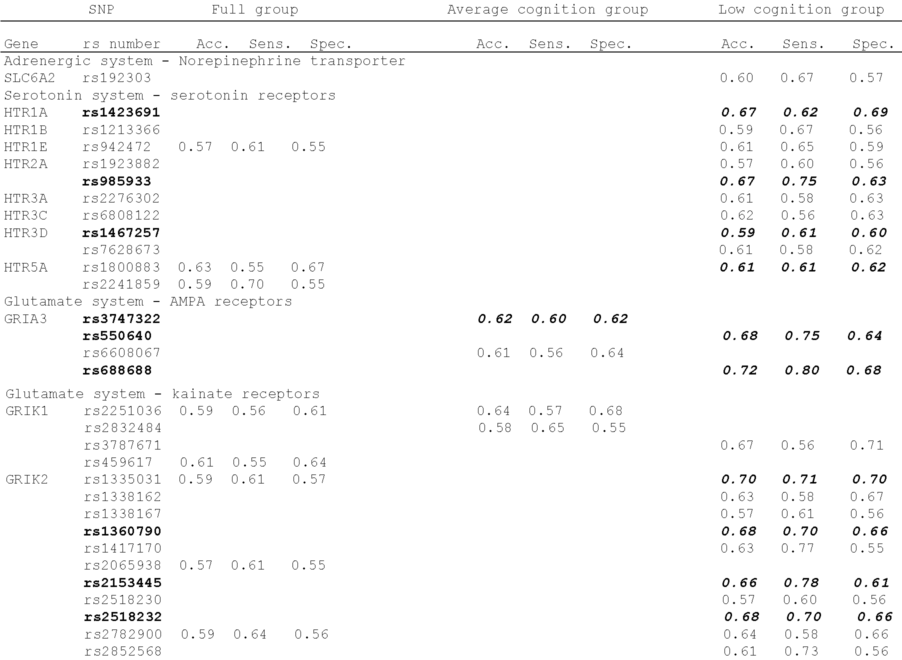

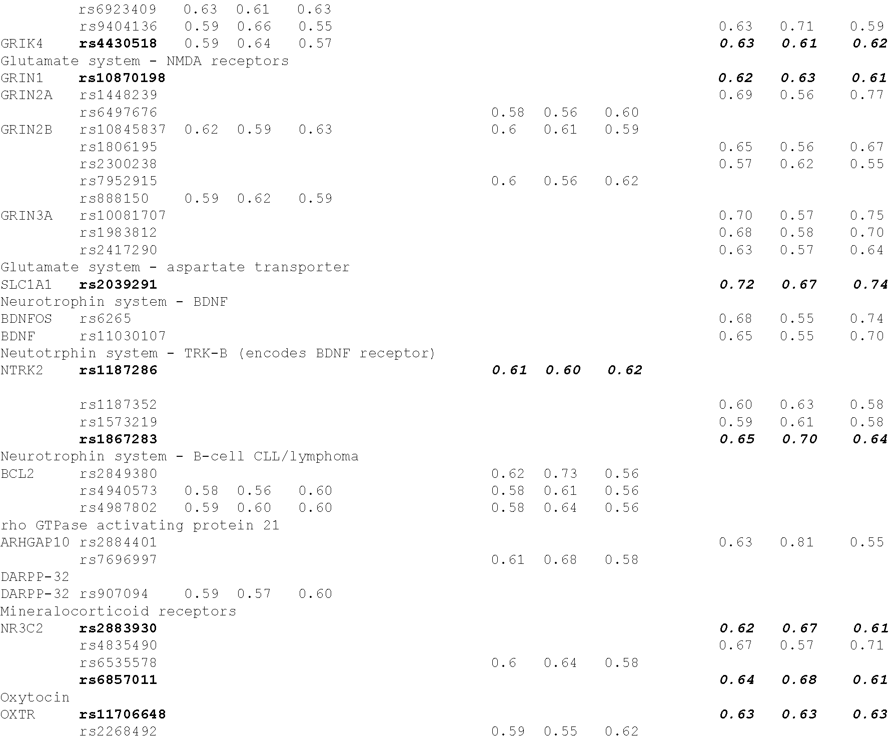

### Method 3

The combination of genomic and cognitive assessment scores can be used to predict treatment outcome with an overall cross-validated accuracy between 60% and 67%, using a multivariate logistic regression technique, with predictors grouped into separate regression models for general cognition, emotional cognition and genomic main and interaction effects, and splitting of the overall sample into sub-groups based on variability of emotional cognition performance.

Design:

This method was designed with the aim of determining whether a ‘replication panel’ of genomic parameters (23 SNPs) with previously linked to treatment outcome in depression could improve the prediction of treatment remission previously established from general and emotional cognition assessment scores alone.

To this end, the most accurate predictions that could be achieved across the maximal number of patients and drug types were identified by exploring different methods of sub-typing patients based on level of baseline performance on specific cognitive tasks (2 scores for each task—average performance and consistency of performance).

The 23 SNPs in the replication panel assessed in this method were ABCB1 rs2032583, ABCB1 rs2235015, BDNFOS rs6265, BDNF rs12273363, BDNF rs1491850, BDNF rs7124442, COMT rs165774, COMT rs4680, CRHR1 rs1876828, CRHR1 rs242939, CRHR2 rs2270007, FKBP5 s1360780, FKBP5 rs3800373, GRIK2 rs2518224, GRIK4 rs1944522, GRIK4 rs1954787, HTR1A rs1364043, HTR2A rs6311, HTR2A rs6313, HTR2A rs6314, HTR2A rs7997012, CRHBP rs10473984, SLC6A2 rs5569.

Step 1:

Stratify sample at pre-treatment baseline in order to capture the heterogeneity of starting conditions. The sample was stratified according to pre-treatment general cognitive and emotional cognitive performance on the subtests, using 2 scores from each task (reflecting average performance level and consistency of performance) and splitting into 3 equal groups of the top, middle, and lowest ⅓ of the group on each of these scores, yielding 26 different scores that were tested with each approach. These splits were assessed based on providing the highest cross-validated accuracy, while applying to the most number of patients and the greatest number of treatment arms.

Step 2:

Run prediction modelling, using multivariate logistic regression models, for the full group and for each treatment arm, for each of the splitting approaches described in Step 1. In order to minimize type 1 error rates, only results yielding cross-validated sensitivity or specificity values that were above 0.60 (with the other above 0.50) were considered for evaluation. All regressions were developed on the full sample then cross-validated 50 times using 80% as training set and 20% as testing set. Results prior to splitting were also considered in order to determine the added benefit of splitting by the group by baseline cognitive performance.

Predictors were established by taking multiple individual measures within each category (general cognition, emotional cognition, and Genomic SNPs) and then running a 5-factor principle components analysis separately for each category, resulting in 5 predictor measures within both categories. This was done in order to maintain sufficient power across while assessing interaction effects by minimizing the number of predictors. Separate predictive regression models were then run for main effects alone and for main and interaction effects together, for general cognition, emotional cognition, and genomic measures run separately and in combination, yielding a total of 14 models for each analysis. This approach was selected due to the large number of predictors, to maximize power to detect small effects. Only the model with the strongest cross-validated results in each case was reported for consideration in the final results.

All results are presented for prediction of symptom remission based on the clinician rated HDRS-17.

Results:

Results for the full group prior to splitting according to baseline cognitive performance, for both the combined treatment arms and each treatment arm considered separately, yielded no results that reached significance based on the criteria of at least one of sensitivity or specificity greater than 60%. Details of all model accuracies are shown in Table 12.

TABLE 12
Outcomes for full sample without splitting
group based on baseline cognition performance

| | | | | |
|---|---|---|---|---|
| | Treatments | | | Venlafaxine- |
| Predictor Sets | combined | Escitalopram | Sertraline | XR |

Main effect models

| | | | | |
|---|---|---|---|---|
| Cognition | .48 | .52 | .55 | .46 |
| Emotion | .50 | .43 | .48 | .48 |
| Genomics | .51 | .54 | .51 | .48 |
| Cognition and | .48 | .53 | .51 | .47 |
| Emotion | | | | |
| Cognition and | .50 | .57 | .49 | .49 |
| Genomics | | | | |
| Emotion and | .50 | .43 | .48 | .48 |
| Genomics | | | | |
| Cognition, | .48 | .53 | .51 | .47 |
| Emotion, | | | | |
| Genomics | | | | |

Combined main and interaction effect models

| | | | | |
|---|---|---|---|---|
| Cognition | .48 | .48 | 55 | .44 |
| Emotion | .51 | .41 | .51 | .53 |
| Genomics | .51 | .56 | .52 | .52 |
| Cognition and | .50 | .51 | .52 | .47 |
| Emotion | | | | |
| Cognition and | .51 | .59 | .55 | .51 |
| Genomics | | | | |
| Emotion and | .51 | .41 | .50 | .53 |
| Genomics | | | | |
| Cognition, | .50 | .51 | .52 | .47 |
| Emotion, | | | | |
| Genomics | | | | |

For all 3 treatment arms, the strongest predictive results were found when splitting the baseline group based on performance variability for the emotion identification task, into 3 groups based on z-score levels, reflecting low or poor performance (a lot of variability), middle range variability of performance, and high or good performance (very little variability; see table 13 for details).

For escitalopram, predictive models with sufficient accuracy were established for the low (poor) and middle performing groups. The best predictors of remission the low group was in the cognition main effect model with a cross-validated accuracy of 62% (sensitivity 53%, specificity 65%), and for the middle group the best predictors were in the combined emotion main and interaction effects model, yielding a cross-validated accuracy of 60% (sensitivity 56%, specificity 64%). The low range sensitivity level for the low performing group of 53% suggests that this model may be poor at accurately predicting who will remit, but has sufficient specificity to accurately predict those who will not remit.

For sertraline, strong predictions were established for full sample, but with different predictors yielding best remission prediction accuracy for the each of the different low, middle and high performing sub-groups. For the low group, the best predictors of remission were in the combined cognition main and interaction effect model, yielding an accuracy of 67% (sensitivity 58%, specificity 71%). For the middle group, the best predictors were in the emotion main effects model, with an accuracy of 66% (sensitivity 77%, specificity 60%), and for the high performing group the combined emotion main and interactions effects model yielded the best prediction of remission, with an accuracy of 64% (sensitivity 67%, specificity 64%).

For venlafaxine-XR, a remission prediction model with sufficient accuracy was only able to be established for the middle range performing group. This model has an accuracy of 61%, with 50% sensitivity and 62% specificity. The low sensitivity but sufficient specificity performance indicates that this model would be most accurate in predicting those who will not remit with the treatment, rather than those who will remit. The model providing this best prediction consisted on the combined cognition and emotion main effects.

TABLE 13
Outcome results for the final prediction model. Data for outcome measures of
HDRS-17 response, and QIDS remission and response, are provided in appendix 15.
Sample split into 3 subgroups by variability of Emotion Identification
performance, at equal 1/3 low, middle and high performance groups

| | | | | |
|---|---|---|---|---|
| Subgroup | | | | |
| split | Accuracy | Sensitivity | Specificity | Predictors |

Full 1008 sample

| | | | | |
|---|---|---|---|---|
| Low | 0.54 | 0.52 | 0.55 | Emotion main effects |
| Middle | 0.58 | 0.61 | 0.56 | Cognition and Emotion main effects |
| High | 0.55 | 0.55 | 0.55 | Emotion main and interaction effects |

Escitalopram

| | | | | |
|---|---|---|---|---|
| Low | 0.62 | 0.53 | 0.65 | Cognition main effects |
| Middle* | 0.61 | 0.63 | 0.59 | Genomic main effects |
| High | 0.63 | 0.61 | 0.65 | Genomic main effects |

Sertraline

| | | | | |
|---|---|---|---|---|
| Low | 0.67 | 0.58 | 0.71 | Cognition main and interaction effects |
| Middle* | 0.66 | 0.77 | 0.60 | Emotion main effects |
| High | 0.64 | 0.66 | 0.64 | Emotion main and interaction effects |

Venlafaxine-XR

| | | | | |
|---|---|---|---|---|
| Low | 0.61 | 0.44 | 0.67 | Cognition main and interaction effects |
| Middle | 0.60 | 0.50 | 0.62 | Cognition and Emotion main effects |
| High | 0.56 | 0.35 | 0.65 | Cognition and SNPs main and interaction |

*For the middle performance group, escitalopram and sertraline prediction were similarly predicted by both Emotional cognition main effects and Genomic main effects, with Genomic main effects being slightly more accurate for escitalopram and Emotional cognition main effects being slightly more accurate for sertraline. The results not presented within the table are Emotional cognition main effects for escitalopram (accuracy 60%, sensitivity 56%, specificity 64%) and Genomic main effects for sertraline (accuracy 59%, sensitivity 54%, specificity 63%).

### Method 4

In a further method of the present invention the combination of genomic and cognitive assessment scores can be used to provide more accurate treatment outcome predictions than for the cognitive assessment scores alone, by separately assessing cognitive predictive features for the 3 different variants of a given genomic SNP.

Design:

This method was designed with the aim of determining whether cognitive predictors of treatment outcome would differ according to the different variants of specific SNPs, and consequently whether stronger predictions can be achieved by considering patients with different genomic make-up separately in cognitive predictive analysis.

Selected examples from the 23 SNP replication panel used in Method 2 were selected for consideration in the current analysis. The following 11 SNPs were chosen for analysis: ABCB1 rs2032583, ABCB1 rs2235015, BDNFOS rs6265, COMT rs4680, CRHR1 rs1876828, CRHR1 rs242939, FKBP5 s1360780, FKBP5 rs3800373, GRIK4 rs1954787, HTR2A rs6313, HTR2A rs7997012.

Run prediction modelling, using multivariate logistic regression models, for the full group and each treatment arm. Predictors from the cognitive assessment were comprised of a single overall performance score for each task, and grouped according to general and emotional cognition task measures, with separate predictive models run for main effects alone and for main and interaction effects together, for general and emotional cognition task measures run separately and in combination, yielding a total of 6 models for each analysis. This approach was selected due to the large number of predictors, to maximize power to detect small effects. Only the model with the strongest cross-validated results in each case was reported for consideration in the final results. In order to minimize type 1 error rates, only results yielding cross-validated sensitivity or specificity values that were above 0.60 (with the other above 0.55) were considered for evaluation. All regressions were developed on the full sample then cross-validated 50 times using 80% as training set and 20% as testing set.

Models were run separately for each of the 3 allele combination variants within each SNP, comprising the homozygous common-common allele variant (C-C), the heterozygous infrequent-common allele variant (I-C), and the homozygous infrequent-infrequent allele variant (I-I). In order to have enough power to run the analyses, analyses were only conducted for SNP variant subtype groups representing greater than 5% of the sample. SNPs for which the homozygous I-I SNP variant could not be assessed due to the group representing less than or equal to 5% of the sample were: ABCB1 rs2032583, ABCB1 rs2235015, BDNFOS rs6265, CRHR1 rs1876828, and CRHR1 rs242939.

All results are presented for prediction of symptom remission based on the clinician rated HDRS-17.

Results:

For the full group without splitting by SNP variants, no significant predictions for treatment outcomes using cognitive predictors alone were found.

For the dopamine system COMT gene SNP rs4680, the significant predictions for escitalopram were found for the homozygous C-C variant only, and significant predictions for venlafaxine-XR were found for the homozygous I-I variant only. For sertraline, significant predictions were found for both groups represent the infrequent allele carriers (I-C and I-I).

For the serotonin receptor HTR2A gene SNPs rs6313 and rs7997012, significant results were found for venlafaxine-XR only for the rs6313 SNP I-C variant, and for sertraline only for the rs7997012 SNP I-I variant.

For the glutamate system kainate receptor GRIK4 gene SNP rs1954787, significant predictions were found for both sertraline and venlafaxine-XR, for each of the homozygous groups (C-C and I-I), but not for the heterozygous group carrying both alleles. However, the predictions for the two homozygous groups were driven by different individual cognitive predictors for sertraline, with the C-C group driven by interactions between the general and emotional cognitive predictors, and the I-I group driven by main effects within the general cognitive predictors alone. For venlafaxine-XR, main effects of general cognitive predictors drove predictions for both homozygous groups.

For both of the ATP-binding cassette ABCB1 gene SNPs tested, significant predictions for sertraline were found for the heterozygous infrequent-common SNP variant only. For the corticotrophine releasing hormone CRHR1 gene SNPs, the C-C and I-C variants assessed also displayed differential cognitive predictors for the escitalopram and sertraline treatment arms.

The BDNFOS and K506 binding protein 5 (FKBP5) SNPs did not yield any significant treatment outcome predictions for any of the treatment arms, for any of the SNP variants assessed.

No significant results were found for the combined treatment arms, for any of the SNP variants assessed.

TABLE 14

| SNP | | | | | | | | | | |
|---|---|---|---|---|---|---|---|---|---|---|
| | SNP | | | | | | | | | |
| | variant | Escitalopram | | | Sertraline | | | Venlafaxine-XR | | |
| SNP label | (% sample) | Acc. | Sens. | Spec. | Acc. | Sens. | Spec. | Acc. | Sens. | Spec. |

All 3 SNP variants assessed
Dopamine system

| | | | | | | | | | | |
|---|---|---|---|---|---|---|---|---|---|---|
| COMT | C-C (30%) | 0.63 | 0.63 | 0.64 | | | | | | |
| rs4680 | I-C (49%) | | | | 0.58 | 0.55 | 0.60 | | | |
| | I-I (21%) | | | | 0.59 | 0.58 | 0.60 | 0.66 | 0.55 | 0.69 |

Serotonin system

| | | | | | | | |
|---|---|---|---|---|---|---|---|
| HTR2A | C-C (33%) | | | | | | |
| rs6313 | I-C (47%) | | | | 0.59 | 0.63 | 0.58 |
| | I-I (20%) | | | | | | |
| HTR2A | C-C (47%) | | | | | | |
| rs7997012 | I-C (40%) | | | | | | |
| | I-I (13%) | 0.64 | 0.69 | 0.64 | | | |

Glutamate system

| | | | | | | | |
|---|---|---|---|---|---|---|---|
| GRIK4 | C-C (31%) | 0.59 | 0.56 | 0.63 | 0.59 | 0.56 | 0.60 |
| rs1954787 | I-C (44%) | | | | | | |
| | I-I (25%) | 0.66 | 0.59 | 0.68 | 0.64 | 0.58 | 0.67 |

2 of 3 SNP variants assessed
ATP-binding cassette

| | | | | |
|---|---|---|---|---|
| ABCB1 | C-C (76%) | | | |
| rs2032583 | I-C (23%) | 0.64 | 0.57 | 0.69 |
| | I-I (1%) n.a. | | | |
| ABCB1 | C-C (62%) | | | |
| rs2235015 | I-C (33%) | 0.59 | 0.60 | 0.59 |
| | I-I (5%) n.a. | | | |

Corticotrophine releasing hormone

| | | | | | | | |
|---|---|---|---|---|---|---|---|
| CRHR1 | C-C (82%) | 0.61 | 0.60 | 0.62 | | | |
| rs242939 | I-C (18%) | | | | 0.61 | 0.59 | 0.63 |
| | I-I (0%) n.a. | | | | | | |
| CRHR1 | C-C (70%) | | | | | | |
| rs1876828 | I-C (25%) | 0.61 | 0.61 | 0.62 | | | |
| | I-I (5%) n.a. | | | | | | |

1. A method of predicting a treatment outcome in a patient with Major Depressive Disorder (MDD) comprising the steps of:
a) assessing a cognitive and/or a genomic parameter in said patient thereby obtaining an assessment score for said parameter; and
b) comparing said assessment score of step a) with a reference set of assessment scores for said parameter to establish a correlation between said assessment score with a corresponding assessment score of the reference set, wherein said corresponding assessment score is linked to a treatment outcome in MDD patients having been treated with a selected antidepressant medication (ADM),
wherein said correlation of step b) is used to predict a treatment outcome for said patient with MDD when treated with the selected ADM.

2. The method according to claim 1, wherein the reference set of assessment scores comprises assessment scores for cognitive and/or genomic parameters identified as statistically significant predictors of treatment outcome in MDD patients having been divided into at least two patient sub-groups based on a comparison of:
assessment scores obtained by said patients for at least one parameter before treatment with a selected ADM; and
assessment scores obtained for said same parameter in a group of subjects without MDD.

3. The method according to claim 2, wherein:
a first cognitive parameter is a predictor of treatment outcome for MDD patients in one of the patient sub-groups when the division into said sub-groups is based on the comparison of at least a second cognitive parameter, or
a genomic parameter is a predictor of treatment outcome for MDD patients in one of the patient sub-groups when the division into said sub-groups is based on the comparison of at least one cognitive parameter.

4. The method according to claim 2, wherein the comparison is based on least two cognitive assessment scores and wherein the patients are divided into sub-groups of “average cognitive performance” and “below average cognitive performance”.

5. The method according to claim 1, wherein treatment outcome is determined by a symptom score according to the clinician-rated 17-item Hamilton Rating Scale for Depression (HRSD₁₇) or according to the self-rated the 16-item Quick Inventory of Depressive Symptomatology-Self Report (QIDS-SR₁₆), wherein a ≧50% decrease of a symptom score determined before treatment with a selected ADM after 8 weeks of treatment with said selected ADM indicate a treatment response and wherein symptom scores of ≦7 on the HRSD₁₇ or of ≦5 on the QIDS-SR₁₆ after 8 weeks of treatment with said selected ADM indicate remission.

6. The method according to claim 1, wherein:
the cognitive parameter is selected from the group consisting of motor coordination assessed by motor tapping test, decision speed assessed by choice reaction time test, verbal memory assessed by memory recall test, working memory assessed by digit span test, cognitive flexibility assessed by verbal interference test, information processing speed assessed by switching of attention test, response inhibition assessed by go/no-go test, attention assessed by continuous performance test, executive function assessed by maze test, emotion identification accuracy and speed assessed by explicit emotion test, and identification and implicit emotion bias assessed by delayed emotion recognition test; or
the genomic parameter is selected from the group single nucleotide polymorphisms (SNPs) shown in FIG. 11.

7. The method according to claim 1, wherein:
the cognitive parameter is selected from the group consisting of motor coordination assessed by motor tapping test, decision speed assessed by choice reaction time test, verbal memory assessed by memory recall test, working memory assessed by digit span test, cognitive flexibility assessed by verbal interference test, information processing speed assessed by switching of attention test, response inhibition assessed by go/no-go test, attention assessed by continuous performance test, executive function assessed by maze test, emotion identification accuracy and speed assessed by explicit emotion test, and identification and implicit emotion bias assessed by delayed emotion recognition test, and
the genomic parameter is selected from the group single nucleotide polymorphisms (SNPs) shown in FIG. 11.

8. The method according to claim 1, wherein the ADM is selected from the group consisting of selective serotonin reuptake inhibitors (SSRIs) and serotonin reuptake inhibitors (SNRIs).

9. The method according to claim 8, wherein the ADM is selected from escitalopram, sertraline and venlafaxine-extended release (venlafaxine-XR).

10. The method according to claim 2, wherein:
the cognitive parameter assessed is emotion identification speed and wherein the MDD patients are divided into sub-groups based on to their assessment scores for attention or executive function, wherein either poor attention or poor executive function predicts symptom remission after 8 weeks of treatment with either escitalopram, sertraline or venlafaxine-XR; or
the cognitive parameter assessed is cognitive flexibility and wherein the MDD patients are divided into sub-groups based on to their assessment scores for delayed recognition of emotion speed, wherein delayed emotion identification speed predicts symptom remission after 8 weeks of treatment with either escitalopram, sertraline or venlafaxine-XR.

11. A method of predicting symptom remission or symptom response in a patient with Major Depressive Disorder (MDD) when treated with a selected ADM comprising the steps of:
a) assessing more than one cognitive and genomic parameter in said patient thereby obtaining an assessment score for each of said parameters;
b) comparing said assessment scores of step a) with a reference set of assessment scores for said parameters to establish a correlation between an assessment score for at least one genomic parameter of step a) with a corresponding assessment score of the reference set,
wherein said corresponding assessment score is linked to symptom remission or symptom response in MDD patients having been treated with a selected antidepressant medication (ADM) based on a regression model with cross-validated sensitivity or specificity of greater than 50% where said MDD patients were divided into patient cognitive performance sub-groups based a comparison of:
assessment scores obtained by said patients for at least one cognitive parameter before treatment with a selected ADM; and
assessment scores obtained for said same cognitive parameter in a group of subjects without MDD;
c) determining into which cognitive performance sub-group said patient falls based on said cognitive assessment scores of step a); and
d) correlating said assessment score for said at least one genomic parameter of step a) with a with a corresponding assessment score of the reference set thereby predicting symptom remission or symptom response in said patient when treated with said selected ADM.

12. The method according to claim 11, wherein the cognitive performance sub-groups are “average cognitive performance” and “below average cognitive performance” sub-groups and wherein said ADM is selected from selective serotonin reuptake inhibitors (SSRIs) and serotonin reuptake inhibitors (SNRIs).

13. The method according to claim 11, wherein:
said regression model is a univariate regression model; or
said regression model is a univariate regression model and includes running separate univariate models for each of the genomic parameters, incorporating cross-validation using a k-fold approach.

14. The method according to claim 11, wherein said genomic parameters are single nucleotide polymorphisms (SNPs).

15. The method according to claim 11, wherein said regression model is a multivariate logistic regression model.

16. The method according to claim 11, wherein said regression model provides a statistical significance of p<0.01.

17. The method according to claim 11, wherein:
said patient falls into said “below average cognition” sub-group and wherein correlating an assessment score for MAOA rs2235186, MAOA rs979605, HTR3D rs9819507, GRIK1 rs2251036, GIRK2 rs1415485, GIRK4 rs2852217, GRIN1 rs4880213, GRIN2B rs1805490 or DARPP-32 rs907094 with a with a corresponding assessment score of the reference set predicts symptom remission in said patient when treated with escitalopram; or
said patient falls into said “average cognition” sub-group and wherein correlating an assessment score for HTR2A rs2770296, GRIK2 rs2518227 or FKBP5 rs136078 with a with a corresponding assessment score of the reference set predicts symptom remission in said patient when treated with escitalopram; or
said patient falls into said “below average cognition” sub-group and wherein correlating an assessment score for HTR2C rs540285, GRIK1 rs363478, GRIN2A rs1650397, GRIN2A rs6416623, GRIN3A rs2050641, BCL2 rs1944420, BCL2 rs2849380, FKBP5 rs1360780, NR3C2 rs1355613, or NR3C2 rs2070951 with a with a corresponding assessment score of the reference set predicts symptom remission in said patient when treated with sertraline; or
said patient falls into said “average cognition” sub-group and wherein correlating an assessment score for ABCB1 rs779319 or NR3C2 rs1512343 with a with a corresponding assessment score of the reference set predicts symptom remission in said patient when treated with sertraline.

18. A method of predicting symptom remission or symptom response in a patient with Major Depressive Disorder (MDD) when treated with a selected ADM comprising the steps of:
a) assessing more than one cognitive parameter in said patient thereby obtaining an assessment score for each of said parameters;
b) comparing said assessment scores of step a) with a reference set of assessment scores for said parameters to establish a correlation between an assessment score for at least one cognitive parameter with a corresponding assessment score of the reference set,
wherein said corresponding assessment score is linked to symptom remission or symptom response in MDD patients having been treated with a selected antidepressant medication (ADM) based on a regression model with cross-validated sensitivity or specificity of greater than 50% where said MDD patients were divided into patient cognitive performance sub-groups based a comparison of:
assessment scores obtained by said patients for at least one cognitive parameter before treatment with a selected ADM; and
assessment scores obtained for said same cognitive parameter in a group of subjects without MDD;
c) determining into which cognitive performance sub-group said patient falls based on said cognitive assessment scores of step a); and
d) correlating said assessment score for said at least one cognitive parameter of step a) with a with a corresponding assessment score of the reference set thereby predicting symptom remission or symptom response in said patient when treated with said selected ADM.

19. The method according to claim 18, wherein the cognitive performance sub-groups are “average cognitive performance” and “below average cognitive performance” sub-groups and wherein said ADM is selected from selective serotonin reuptake inhibitors (SSRIs) and serotonin reuptake inhibitors (SNRIs).

20. The method according to claim 18, wherein:
said regression model is a univariate regression model; or
said regression model is a univariate regression model and includes running separate univariate models for each of the cognitive parameters, incorporating cross-validation using a k-fold approach.

21. The method according to claim 18, wherein said regression model is a multivariate logistic regression model.

22. The method according to claim 18, wherein said regression model provides a statistical significance of p<0.01.

23. A method of treating Major Depressive Disorder (MDD) in a patient, wherein said MDD is associated with a cognitive and/or a genomic parameter, said method comprising the steps of:
a) assessing said cognitive and/or a genomic parameter in said patient thereby obtaining an assessment score for said parameter;
b) comparing said assessment score with a reference set of assessment scores for said parameter to establish a correlation between said assessment score obtained in step a) with a corresponding assessment score of the reference set;
c) selecting an antidepressant medication (ADM) based on said correlation of step b), and wherein said correlation is established for a parameter linked to beneficial treatment outcome in MDD patients having been treated with said ADM; and
d) administering said ADM selected in c) to said patient to treat said MDD.
